(19) United States
(12) Patent Application Publication
(10) Pub. No.: US 2025/0236633 A1
(43) Pub. Date: Jul. 24, 2025

(54) SYNTHESIS OF ISOMERICALLY PURE POLYOL-BASED PHOSPHORAMIDITES

(71) Applicant: Roche Sequencing Solutions, Inc., Pleasanton, CA (US)

(72) Inventors: Brent Banasik, Seattle, WA (US); Julian Andres Diaz Corral, Seattle, WA (US); Aaron Jacobs, Seattle, WA (US); Lukas Jud, Penzberg (DE); Hannes Kuchelmeister, Munchen (DE); Melud Nbavi, Seattle, WA (US); Toni Pfaffeneder, Gauting (DE); Sona Simonyiova, Muenchen (DE); John C. Tabone, Kirkland, WA (US); Wilma Thuer, Starnberg (DE)

(73) Assignee: Roche Sequencing Solutions, Inc., Pleasanton, CA (US)

(21) Appl. No.: 18/895,484

(22) Filed: Sep. 25, 2024

Related U.S. Application Data
(63) Continuation of application No. PCT/EP2023/057893, filed on Mar. 28, 2023.
(60) Provisional application No. 63/324,272, filed on Mar. 28, 2022.
(60) Provisional application No. 63/353,942, filed on Jun. 21, 2022.

(51) Int. Cl. C07F 9/6518; C07C 47/277; C07D 317/22; C07F 7/08; C07F 9/24; C08G 65/34; C08G 79/04

(52) U.S. Cl. CPC C07F 9/6518; C07C 47/277; C07D 317/22; C07F 7/0812; C07F 9/2458; C08G 65/34; C08G 79/04

(57) ABSTRACT

The present disclosure relates to a process for the regiochemically and enantiomerically controlled synthesis of phosphoramidite-containing monomers, and to intermediate products of this process. In some embodiments, the phosphoramidite-containing monomers or their precursors are regioisomerically and/or enantiomerically pure and may be polymerized into polymers or copolymers.

### CROSS REFERENCES TO RELATED APPLICATIONS

The present application is a continuation of International Patent Application No. PCT/EP2023/057893 filed Mar. 28, 2023, which claims the benefit of the filing date of U.S. Patent Application No. 63/353,942 filed on Jun. 21, 2022, and also claims the benefit of the filing date of U.S. Patent Application No. 63/324,272 filed on Mar. 28, 2022, the disclosures of which are hereby incorporated by reference herein in their entireties.

### BACKGROUND OF THE DISCLOSURE

The “Sequencing by Expansion” (SBX) protocol, developed by Stratos Genomics (see, e.g., Kokoris et al., U.S. Pat. No. 7,939,259, “High Throughput Nucleic Acid Sequencing by Expansion”) is based on the polymerization of highly modified, non-natural nucleotide analogs referred to as “XNTPs.” In general, SBX uses biochemical polymerization to transcribe the sequence of a DNA template onto a measurable polymer called an “Xpandomer.” The transcribed sequence is encoded along the Xpandomer backbone in high signal-to-noise reporters that are separated by ˜10 nm and which are designed for high-signal-to-noise, well-differentiated responses. Xpandomers can facilitate several next generation DNA sequencing detection technologies and are well suited to nanopore sequencing.

XNTPs are expandable, 5′ triphosphate modified non-natural nucleotide analogs compatible with template dependent enzymatic polymerization. XNTPs and their constituent components are described in PCT Publication No. WO/2020/236526, the disclosure of which is hereby incorporated by reference herein in its entirety. Essentially, the XNTPs have the structure:

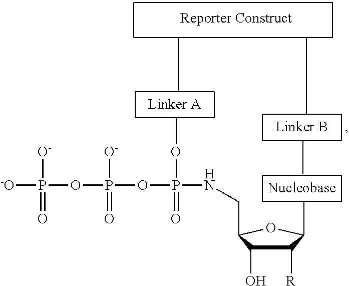

wherein R is OH or H; nucleobase is adenine, cytosine, guanine, thymine, uracil or a nucleobase analog; reporter construct is a polymer having a first end and a second end, and includes, in series from the first end to the second end, a first reporter code, a symmetrical chemical brancher bearing a translocation control element, and a second reporter code; linker A joins the oxygen atom of an alpha phosphoramidate group to the first end of the reporter construct; and linker B joins the nucleobase to the second end of the reporter construct.

The polymeric reporter construct and its constituent elements include repeating monomeric units derived from phosphoramidite-containing monomers (non-limiting examples of phosphoramidite-containing monomers and their synthesis are described in PCT Publication No. WO/2020/236526). For instance, the translocation control element may be a polymer or copolymer derived from repeating phosphoramidite-containing monomeric units (e.g., 1,3-O-bis(phosphodiester)-2S-O-mPEG4-propane). Likewise, the first and second reporter codes may be a polymer or copolymer derived from repeating phosphoramidite-containing monomeric units.

Nanopores used in sequencing are chiral environments. Regioisomeric and enantiomeric impurities in the oligomers, polymers, or copolymers included within any component of an XNTP may have an effect on signal generation (base calling) and processing rates in nanopore sequencing. Regioisomerically and enantiomerically pure phosphoramidite-containing monomers are desirable in the synthesis of oligomers, polymers, or copolymers for incorporation into XNTP.

### BRIEF SUMMARY OF THE DISCLOSURE

Applicant has developed a method of synthesizing regioisomerically and enantiomerically pure phosphoramidite-containing monomers and their intermediates. Applicant has unexpectedly discovered that the selection of certain protecting groups facilitate the synthesis of such regioisomerically pure phosphoramidite-containing monomers in high yield. In particular, Applicant has developed protecting group strategies which utilize non-migrating protecting groups to yield isomerization-free products and which are stable throughout the course of downstream transformation and deprotection reactions. Moreover, Applicant has surprisingly discovered that the protecting groups employed are entirely removable without pronounced decomposition or transformation of other functional groups of the protected molecule, such as base-labile esters, acid-labile trityl-protected alcohols, or oxidation-sensitive PEG-linkers.

In view of the foregoing, a first aspect of the present disclosure is a compound having the structure of any one of Formulas (IA) and (IB):

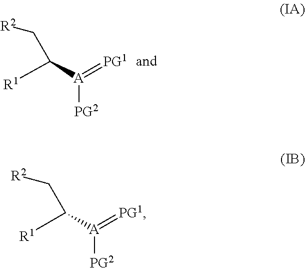

wherein
R¹ is —OH, —O—R^{w}—Z, where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, or —CH₂-Het, where “Het” is a substituted or unsubstituted 5-membered heterocyclic moiety;
R² is —OH, —O-trityl or a derivative or analog thereof, —O-9-phenylthioxanthyl (pixyl) or a derivative or analog thereof, —O-2-(2-nitrophenyl)prop-1-oxycarbonyl (“NPPOC”), —O-(2-nitrobenzyl) or a derivative or analog thereof, —O-(1-(2-nitrophenyl)ethyl) or a derivative or analog thereof, —O-1-(2-fluorophenyl)-4-methoxypiperidin-4-yl, —O-[(chloro-4-methyl)phenyl]-4′-methoxypiperidin-4-yl, tetrahydropyranyl ether, ethoxyethyl ether, methallyl ether, prenyl ether, or methoxymethyl ether;
A is CH;
PG² is H;

PG¹ is

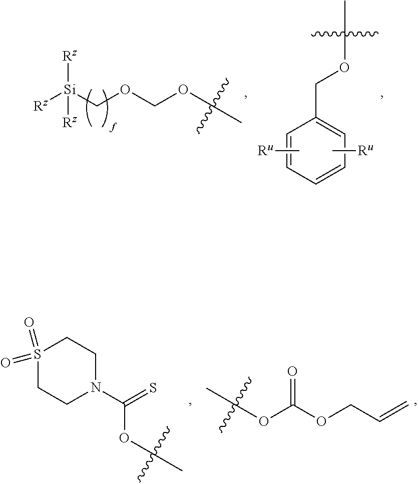

—O—CH₂—S—CH₃, —O—CH₂—N₃, or —O—CH₂—CH═CH₂; or
PG¹-A-PG² taken together form C(O)H, C(O)OMe, —C(O)OT,

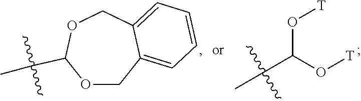

T is an C₁-C₆ branched or unbranched alkyl group;
f is 0 or 2;
each R^{u} is independently H, —O—C₁-C₄ alkyl or —C₁-C₄ alkyl; and
each R^{z} is independently a branched or unbranched C₁-C₆ alkyl group.

In some embodiments, when PG¹-A-PG² together form C(O)H, C(O)OMe, or

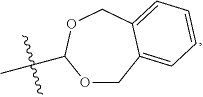 [SMILES: *C1OCc2ccccc2CO1]

R¹ and R² are both not —OH.

In some embodiments, R² is:

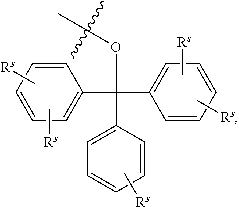

where each R^{s} is independently H, —O—C₁-C₄ alkyl or —C₁-C₄ alkyl. In some embodiments, R² is:

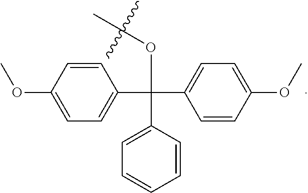

In some embodiments, PG¹ is

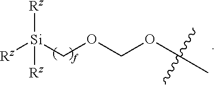

In some embodiments, PG¹ is

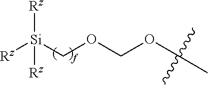

and f is 2.

In some embodiments, PG¹ is

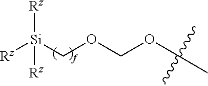

and f is 0.

In some embodiments, PG¹ is

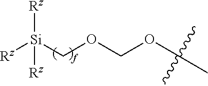

### and R¹ is —OH.

In some embodiments, PG¹ is

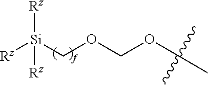

and R¹ is —O—R^{w}—Z.

In some embodiments, PG¹ is

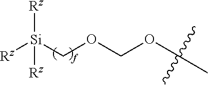

and Z is alkyl, alkenyl, alkynyl, —CH₂-alkynyl, or acyl.

In some embodiments, PG¹ is

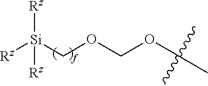

and Z is alkyl.

In some embodiments, PG¹ is

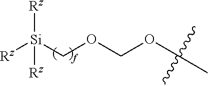

and Z is alkynyl or —CH₂-alkynyl.

In some embodiments, PG¹ is

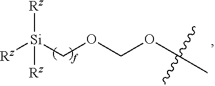

and wherein each R^{z} is a C₁-C₆ branched or unbranched alkyl group. In some embodiments, PG¹ is

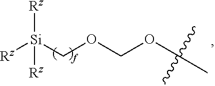

and wherein each R^{z} is a C₁-C₃ branched or unbranched alkyl group.

In some embodiments, PG¹ is:

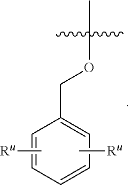

In some embodiments, PG¹ is

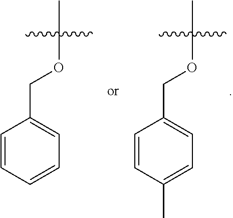

In some embodiments, PG¹ is:

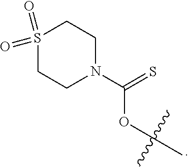 [SMILES: *OC(=S)N1CCS(=O)(=O)CC1 ; "."]

In some embodiments, PG¹ is —O—CH₂—N₃.

In some embodiments, PG¹ is —O—CH₂—S—CH₃. In some embodiments, PG¹ is —O—CH₂—S—CH₃; and Z is alkynyl or —CH₂-alkynyl. In some embodiments, PG¹ is —O—CH₂—S—CH₃; and Z is —CH₂-Het.

In some embodiments, PG¹ is —O—CH₂—CH═CH₂. In some embodiments, PG¹ is —O—CH₂—CH═CH₂; and Z is alkynyl or —CH₂-alkynyl. In some embodiments, PG¹ is —O—CH₂—CH═CH₂; and Z is —CH₂-Het.

In some embodiments, PG¹-A-PG² together form C(O)H, C(O)OMe, —C(O)OT,

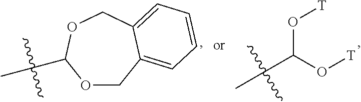

In some embodiments, R¹ and R² am both —OH, and wherein PG is:

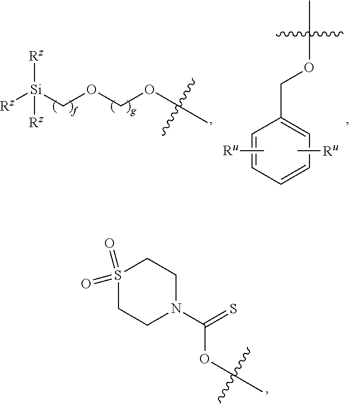

—O—CH₂—S—CH₃, —O—CH₂—N₃, or —O—CH₂—CH═CH₂.

A second aspect of the present disclosure is a compound having the structure of any one of Formulas (IIIA) and (IIIB):

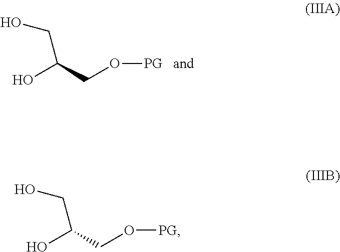

wherein
PG is

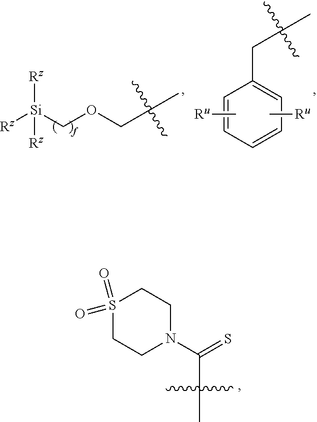

—CH₂—S—CH₃, —CH₂—N₃, or —CH₂—CH═CH₂;
f is 0 or 2;
each R^{u} is independently H, —O—C₁-C₄ alkyl or —C₁-C₄ alkyl; and
each R^{z} is independently a branched or unbranched C₁-C₆ alkyl group.

A third aspect of the present disclosure is compound having the structure of any one of Formulas (IVA) and (IVB):

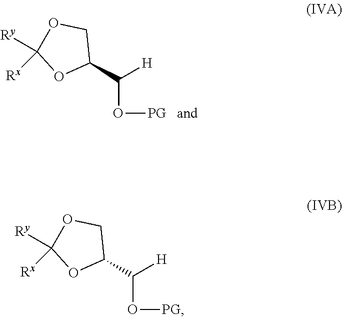

wherein
PG is

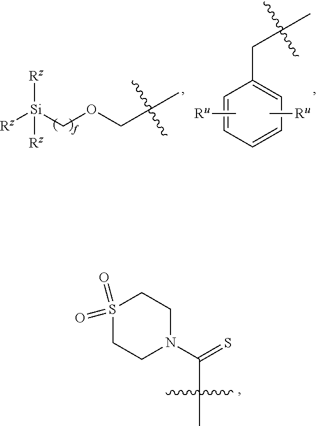

—CH₂—S—CH₃, —CH₂—N₃, or —CH₂—CH═CH₂;
f is 0 or 2;
each R^{u} is independently H, —O—C₁-C₄ or —C₁-C₄;
each R^{z} is independently a branched or unbranched C₁-C₆ alkyl group; and
R^{x} and R^{y} are independently a branched or unbranched C₁-C₄ alkyl group.

A fourth aspect of the present disclosure is a compound having the structure of any one of Formulas (VA) and (VB):

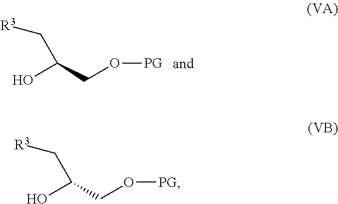

wherein
PG is

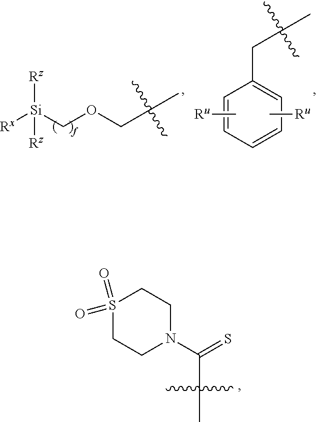

—CH₂—S—CH₃, —CH₂—N₃, or —CH₂—CH═CH₂;
R³ is

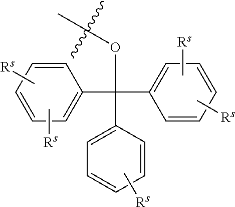

or —O-9-phenylthioxanthyl or a derivative or analog thereof;
f is 0 or 2;
each R^{u} is independently H, —O—C₁-C₄ alkyl or —C₁-C₄ alkyl;
each R^{s} is independently H, —O—C₁-C₄ alkyl or —C₁-C₄ alkyl; and
each R^{z} is independently a branched or unbranched C₁-C₆ alkyl group.

A fifth aspect of the present disclosure is compound having the structure of any one of Formulas (VIIA) and (VIIB):

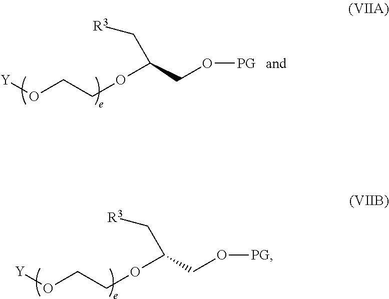

wherein
PG is

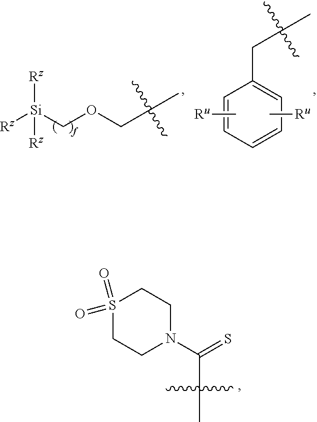

—CH₂—S—CH₃, —CH₂—N₃, or —CH₂—CH₂═CH₂;
R³ is

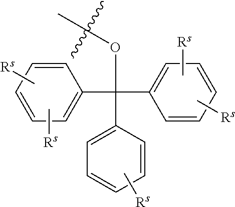

or —O-9-phenylthioxanthyl or a derivative or analog thereof;
Y is alkyl, alkenyl, alkynyl, acyl, —CH₂-alkynyl, a “click functional group,” -Het, or —CH₂-Het, where “Het” is a substituted or unsubstituted 5-membered heterocyclic moiety;
each R^{u} is independently H, —O—C₁-C₄ alkyl or —C₁-C₄ alkyl;
each R^{s} is independently H, —O—C₁-C₄ alkyl or —C₁-C₄ alkyl;
each R^{z} is independently a branched or unbranched C₁-C₆ alkyl group;
e is an integer ranging from between 1 to about 36; and
f is 0 or 2.

A sixth aspect of the present disclosure is compound having the structure of any one of Formulas (XA) and (XB):

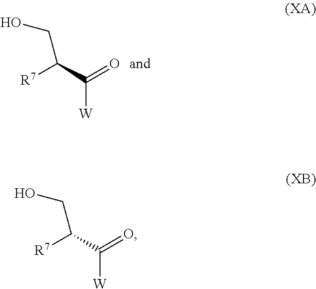

W is H, —O—CH₃ or —O-T, where T is an C₁-C₆ branched or unbranched alkyl group;
R⁷ is —O—R^{w}—Z, where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, or —CH₂-Het, where “Het” is a substituted or unsubstituted 5-membered heterocyclic moiety.

In some embodiments, R⁷ has any one of Formulas (VIIIA) and (VIIIB):

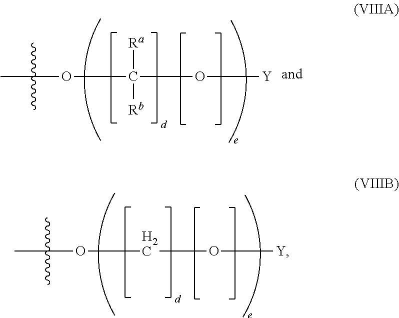

R^{a} and R^{b} are independently H or a C₁-C₄ alkyl;
Y is alkyl, alkenyl, alkynyl, acyl, —CH₂-alkynyl, a “click functional group,” -Het, or —CH₂-Het, where “Het” is a substituted or unsubstituted 5-membered heterocyclic moiety;
d is an integer ranging from between 1 to about 10; and
e is an integer ranging from between 1 to about 36.

A seventh aspect of the present disclosure is compound having the structure of any one of Formulas (XIA) and (XIB):

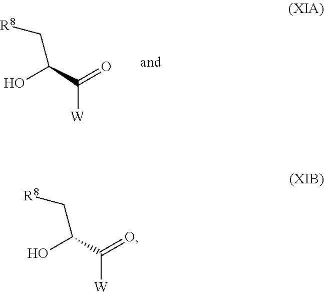

W is H, —O—CH₃ or —O-T, where T is an C₁-C₆ branched or unbranched alkyl group; and

R⁸ is —OH, —O-trityl or a derivative or analog thereof, —O-9-phenylthioxanthyl (pixyl) or a derivative or analog thereof, —O-2-(2-nitrophenyl)prop-1-oxycarbonyl (“NPPOC”), —O-(2-nitrobenzyl) or a derivative or analog thereof, —O-(1-(2-nitrophenyl)ethyl) or a derivative or analog thereof, —O-1-(2-fluorophenyl)-4-methoxypiperidin-4-yl, —O-[(chloro-4-methyl)phenyl]-4′-methoxypiperidin-4-yl, tetrahydropyranyl ether, ethoxyethyl ether, methallyl ether, prenyl ether, or methoxymethyl ether.

An eighth aspect of the present disclosure is a compound selected from:

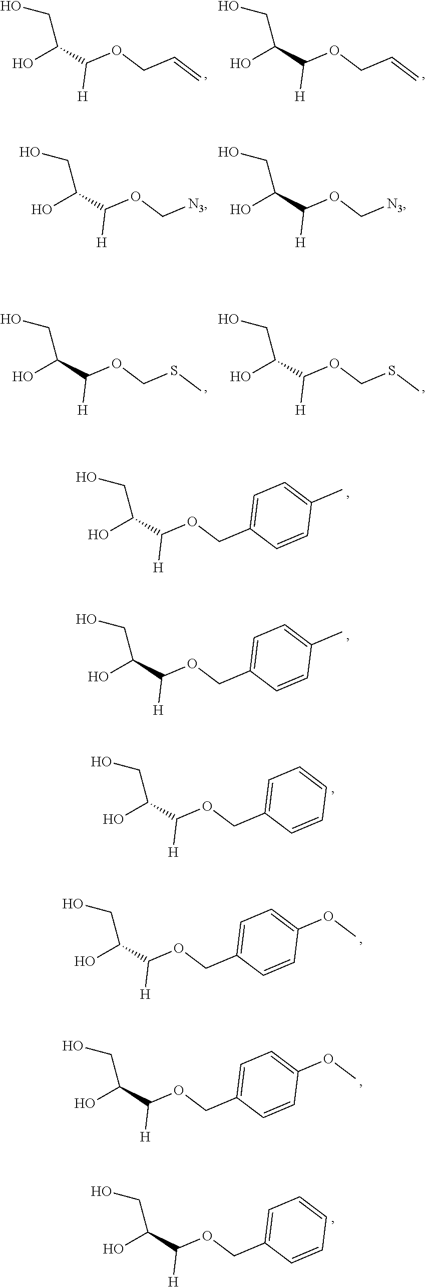

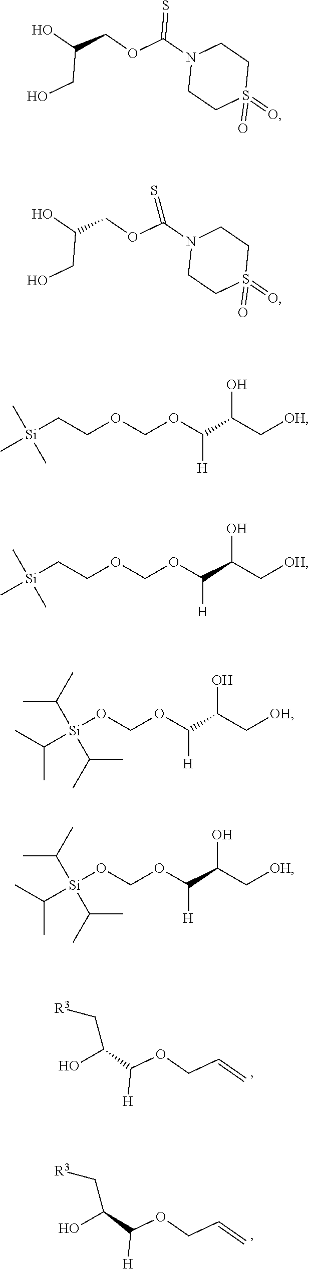

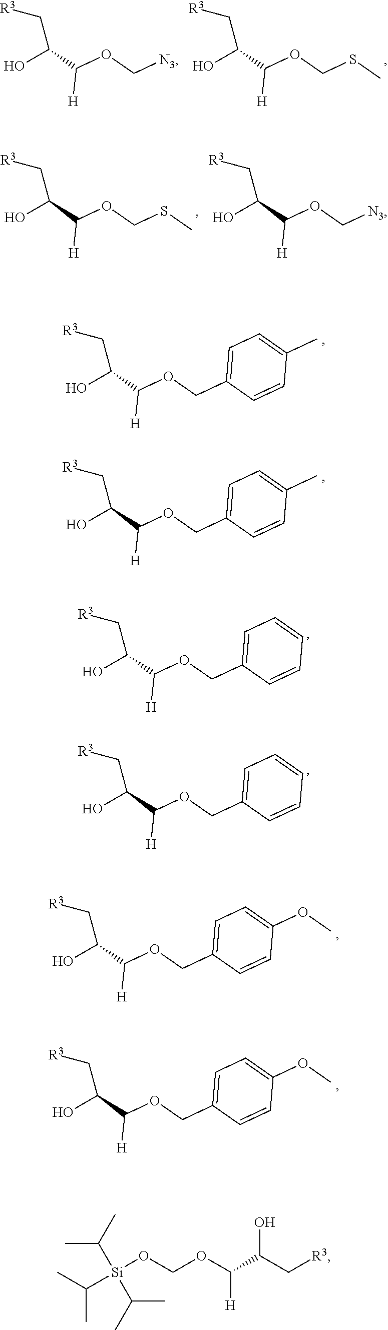

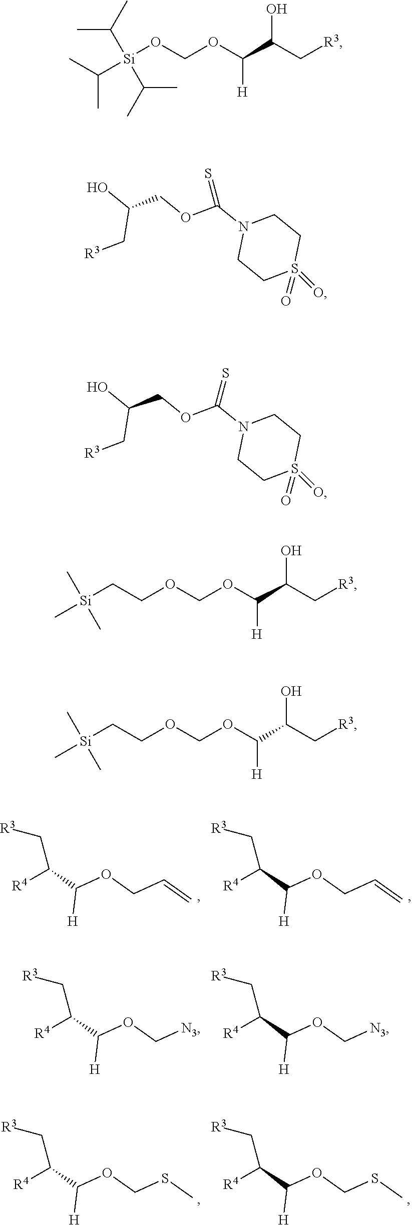

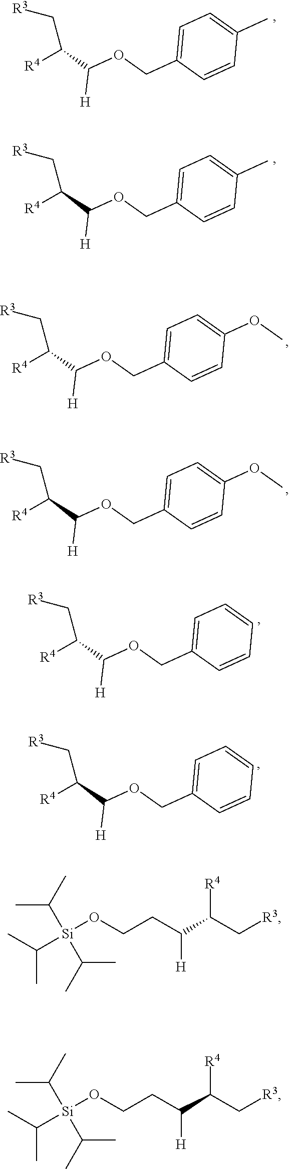

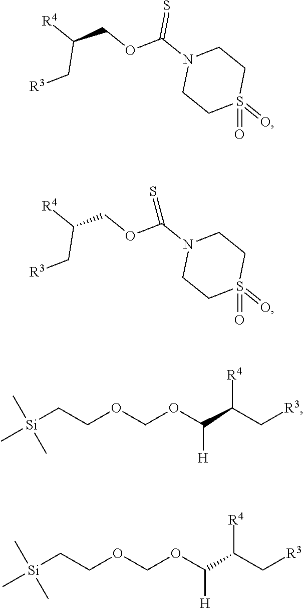

and,

where R³ is 4,4′-dimethoxytrityl ether, 4-methoxytrityl ether, or —O-(9-phenylthioxanthyl); R⁴ is —O-PEG₃-Y or —O-PEG₄-Y, and Y is methyl, —CH═CH₂, or

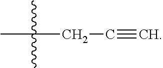 [SMILES: *CC#C]

A ninth aspect of the present disclosure is a compound selected from:

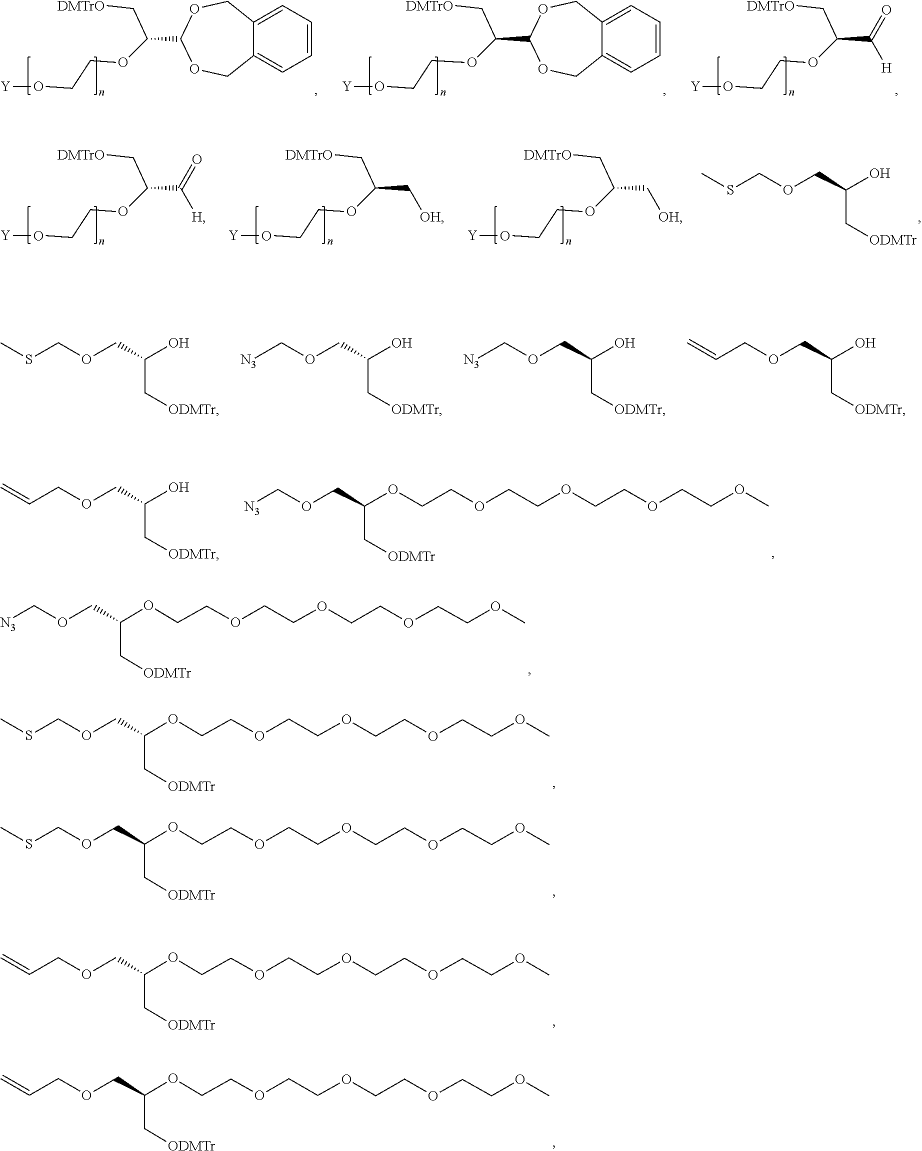

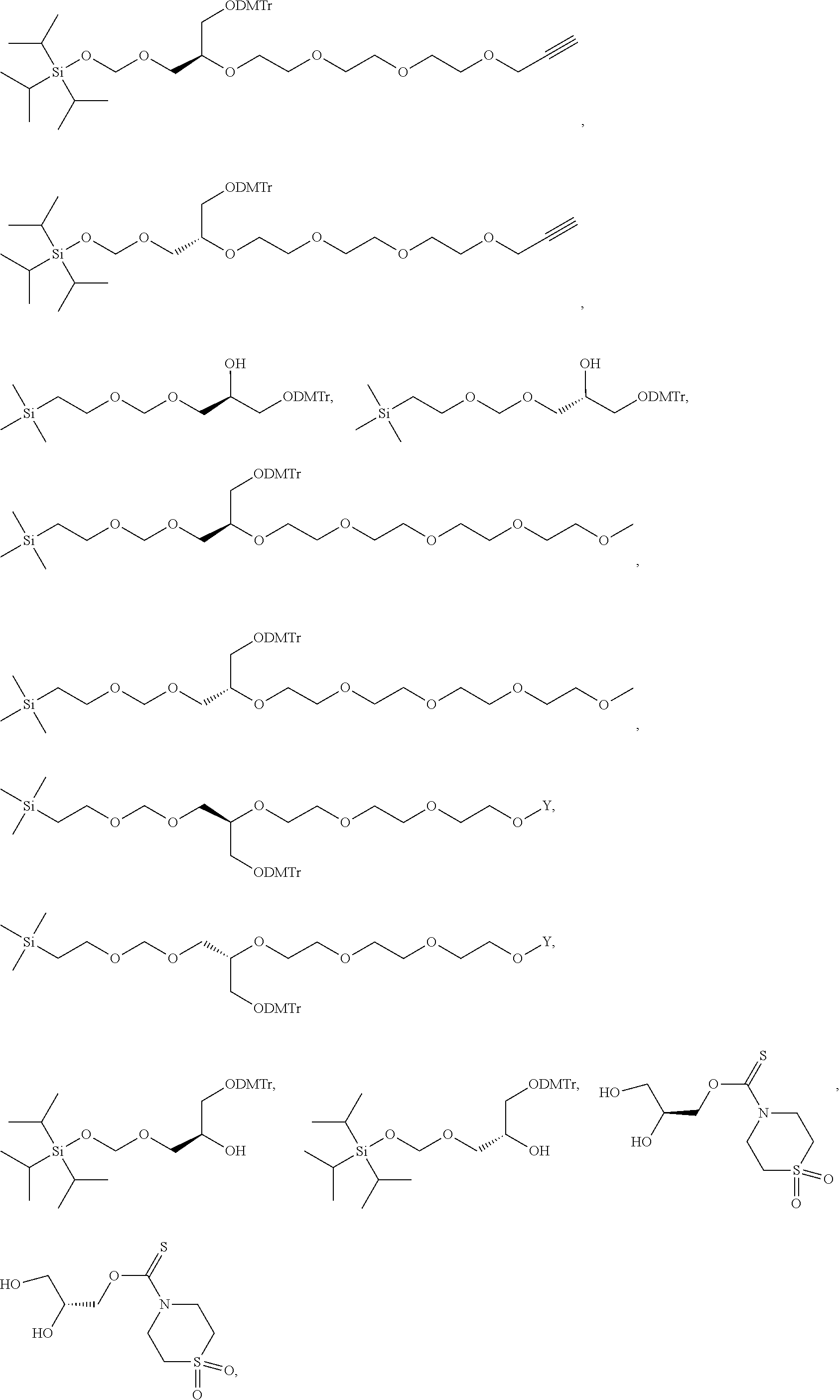

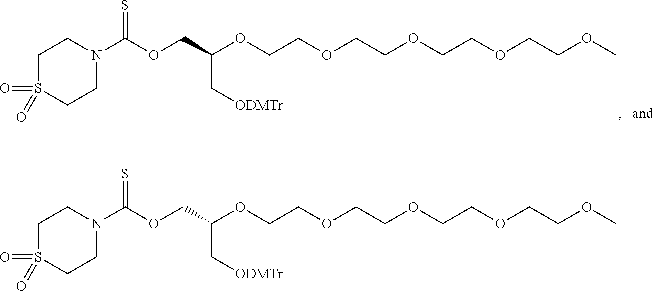

where n ranges from 1 to 24; Y is alkyl, alkenyl, alkynyl, acyl, —CH₂-alkynyl, a “click functional group,” -Het, or —CH₂-Het, where “Het” is a substituted or unsubstituted 5-membered heterocyclic moiety; and -ODMTr is 4,4′-dimethoxytrityl ether.

A tenth aspect of the present disclosure is a compound selected from:

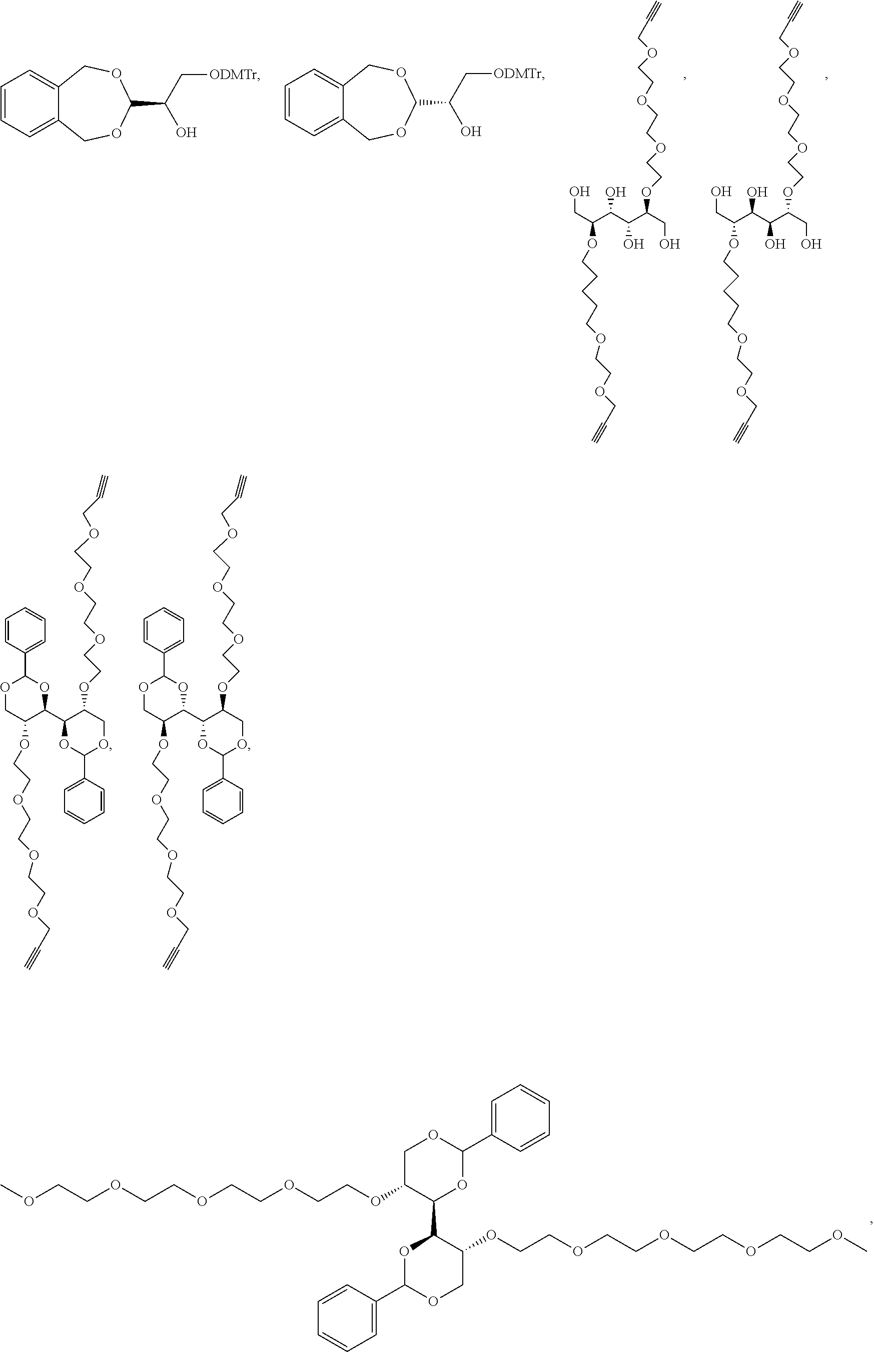

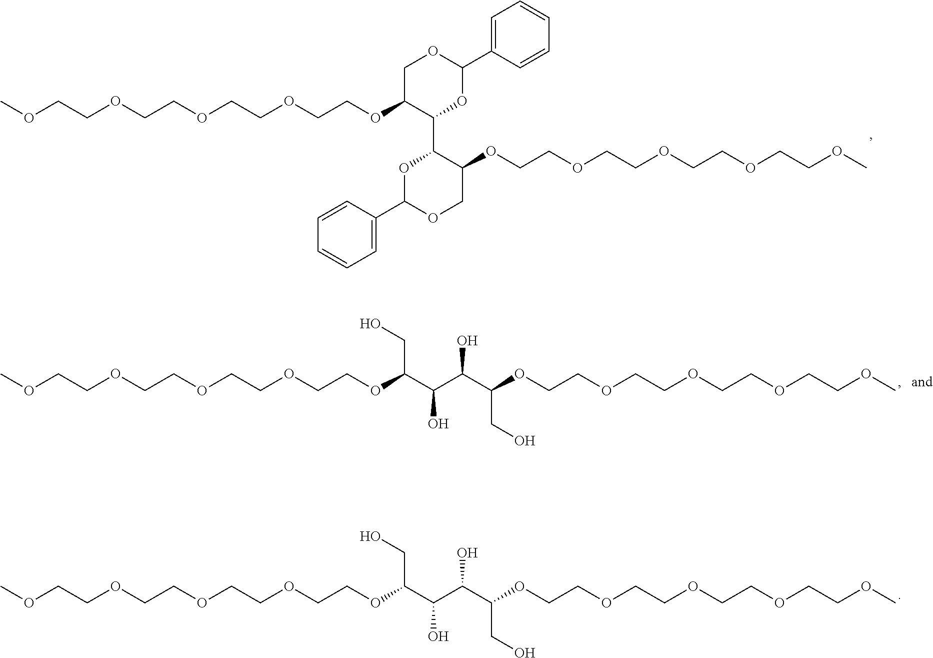

where ODMTr is 4,4′-dimethoxytrityl ether.

An eleventh aspect of the present disclosure is a compound having any one of Formulas (XIIIA) or (XIIIB):

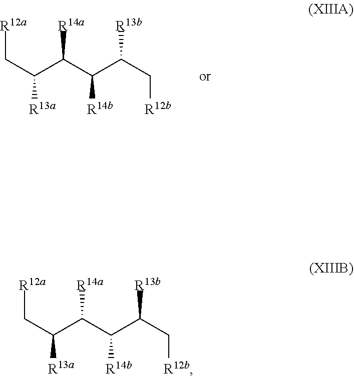

wherein
R^{12a} and R^{12b} are independently —OH, —O-trityl or a derivative or analog thereof, —O-9-phenylthioxanthyl (pixyl) or a derivative or analog thereof, —O-2-(2-nitrophenyl)prop-1-oxycarbonyl (“NPPOC”), —O-(2-nitrobenzyl) or a derivative or analog thereof, —O-(1-(2-nitrophenyl)ethyl) or a derivative or analog thereof, tetrahydropyranyl ether, ethoxyethyl ether, methallyl ether, prenyl ether, or methoxymethyl ether;
R^{13a} and R^{13b} are independently —O—R^{w}—Z, where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, or —CH₂-Het, where “Het” is a substituted or unsubstituted 5-membered heterocyclic moiety;
R^{14a} and R^{14b} are —OH;
or where R^{12a} and R^{14a} and/or R^{12b} and R^{14b} taken together may be

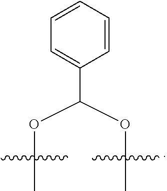 [SMILES: *OC(O*)c1ccccc1]

A twelfth aspect of the present disclosure is a compound having any one of Formulas (IXA) and (IXB), respectively:

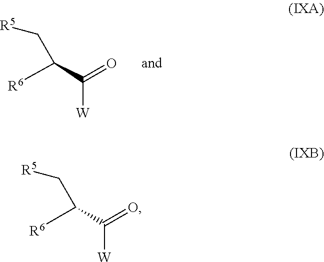

where
W is H, —O—CH₃, or —O-T, where T is an C₁-C₆ branched or unbranched alkyl group;
R⁵ is —OH, —O-trityl or a derivative or analog thereof, —O-9-phenylthioxanthyl (pixyl) or a derivative or analog thereof, —O-2-(2-nitrophenyl)prop-1-oxycarbonyl (“NPPOC”), —O-(2-nitrobenzyl) or a derivative or analog thereof, —O-(1-(2-nitrophenyl)ethyl) or a derivative or analog thereof, tetrahydropyranyl ether, ethoxyethyl ether, methallyl ether, prenyl ether, or methoxymethyl ether;
R⁶ is —OH, —O—R^{w}—Z, where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, or —CH₂-Het, where “Het” is a substituted or unsubstituted 5-membered heterocyclic moiety;
provided that R⁵ and R⁶ are both not —OH.

A thirteenth aspect of the present disclosure is a regioisomerically and enantiomerically pure phosphoramidite-containing monomer derived from any of the compounds of the first through twelfth aspects of the present disclosure. In some embodiments, the present disclosure provides for an oligomer, a polymer, or a copolymer derived from one or more regioisomerically and enantiomerically pure phosphoramidite-containing monomers, where the one or more regioisomerically and enantiomerically pure phosphoramidite-containing monomers are themselves derived from any of the compounds of the first through tenth aspects of the present disclosure.

### BRIEF DESCRIPTION OF THE FIGURES

For a general understanding of the features of the disclosure, reference is made to the drawings. In the drawings, like reference numerals have been used throughout to identify identical elements.

FIG. 1 is a ³¹P NMR spectrum showing a regioisomeric impurity produced during the course of the synthesis outlined in Scheme 2.

FIG. 2 is a ³¹P NMR spectrum showing the product recovered according to the process set forth in Example 1.

FIG. 3 is a ³¹P NMR spectrum showing the product recovered according to the process set forth in Example 2.

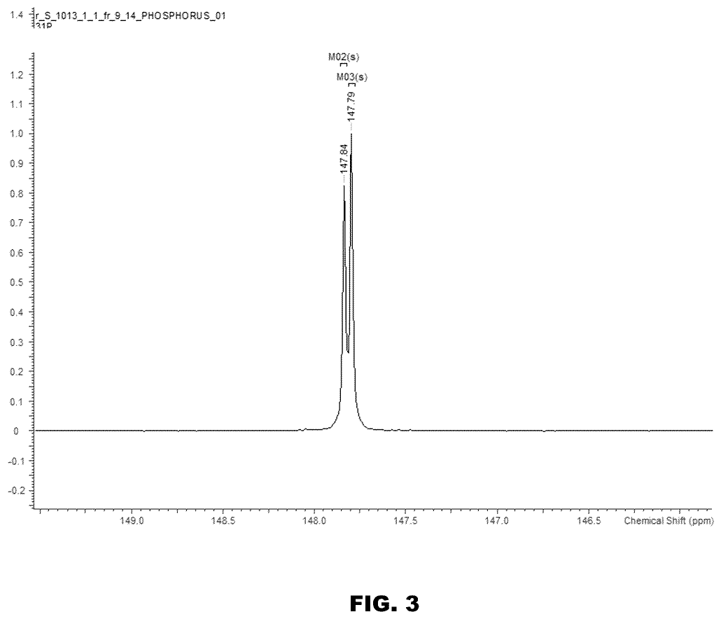

FIG. 4 is a ³¹P NMR spectrum showing the product recovered according to the process set forth in Example 3.

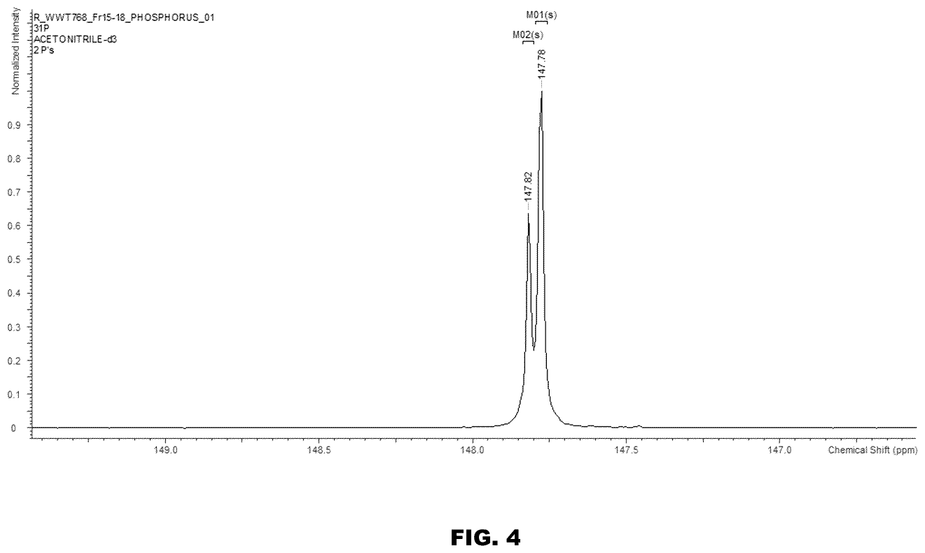

FIG. 5 is a ³¹P NMR spectrum showing the product recovered according to the process set forth in Example 4.

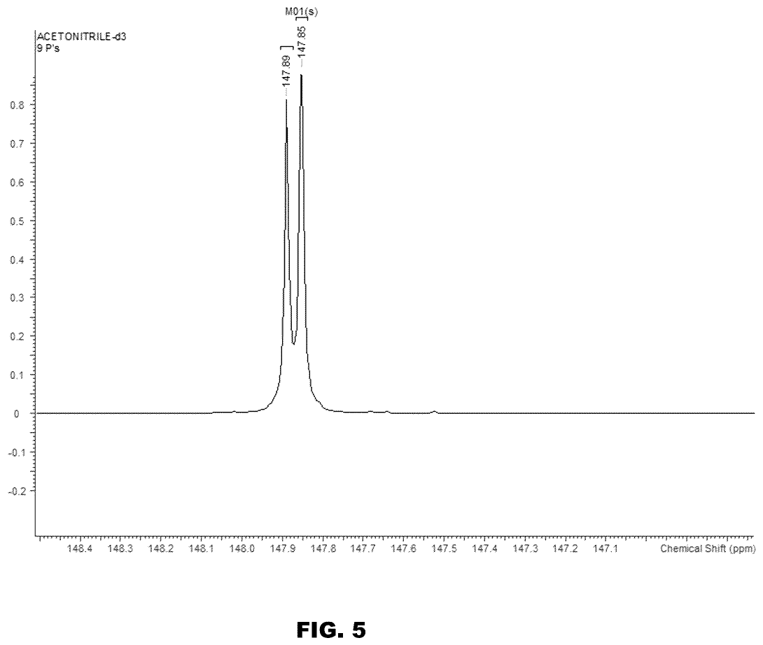

FIG. 6 is a ³¹P NMR spectrum showing the product recovered according to the process set forth in Example 6.

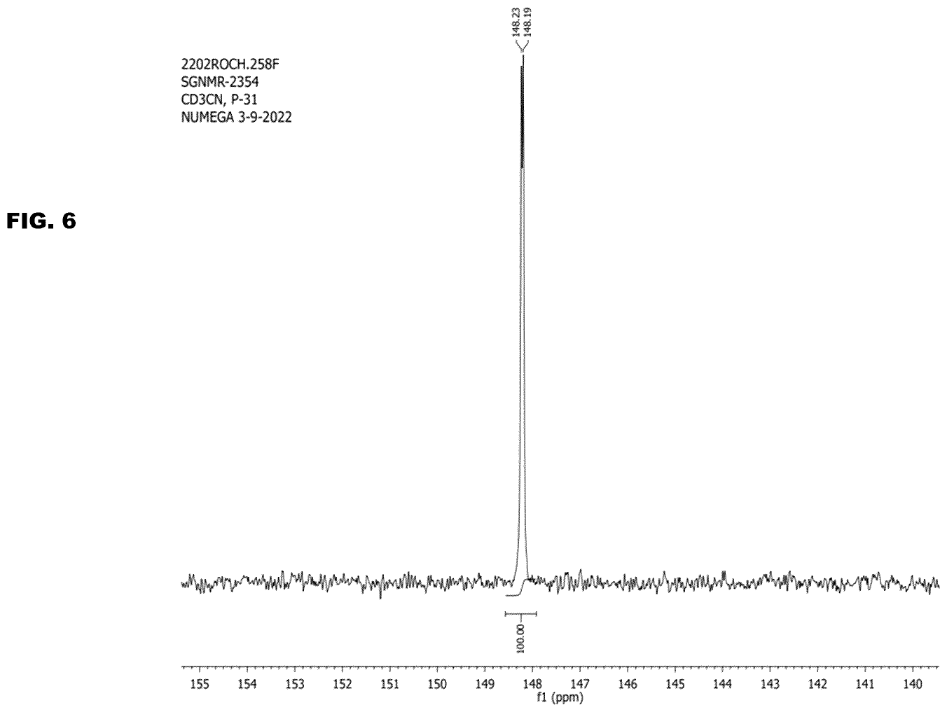

FIG. 7 is a ³¹P NMR spectrum showing the product recovered according to the process set forth in Example 14.

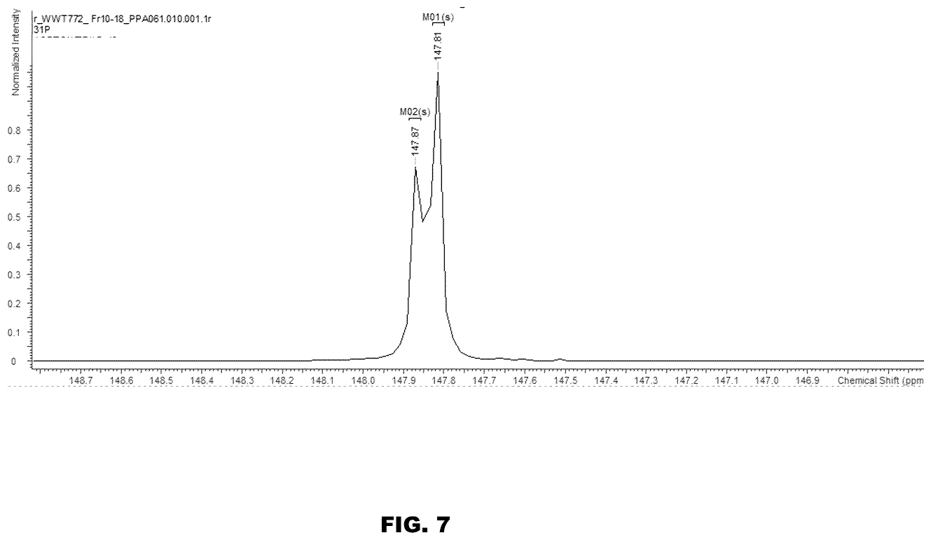

FIG. 8 is a ³¹P NMR spectrum showing the product recovered according to the process set forth in Example 15.

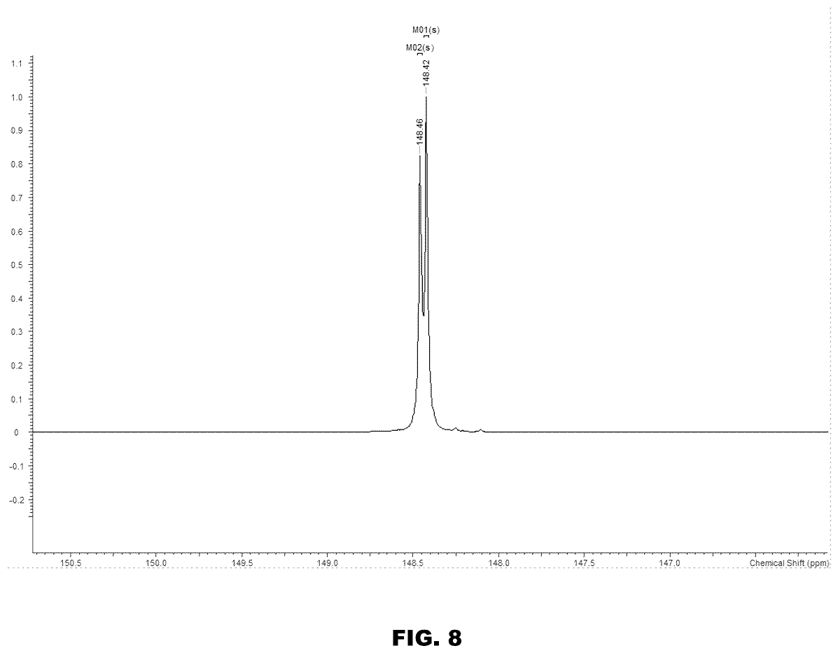

FIG. 9 is a ³¹P NMR spectrum showing the product recovered according to the process set forth in Example 18.

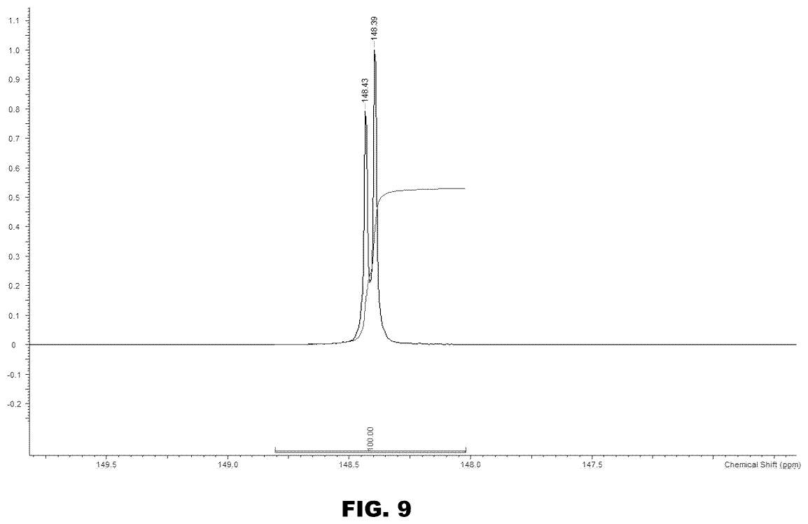

FIG. 10 is a ³¹P NMR spectrum showing the product recovered according to the process set forth in Example 19.

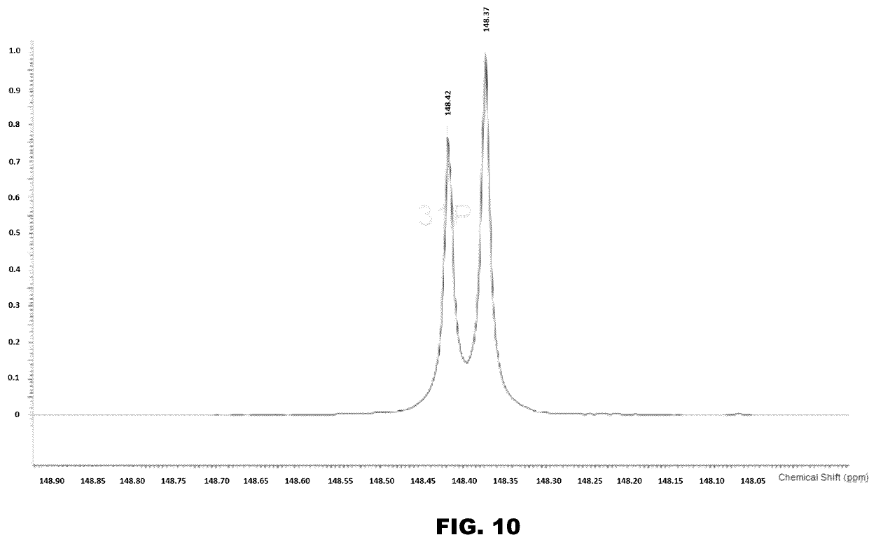

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of.” “Consisting essentially of,” when used in the claims, shall have its ordinary meaning as used in the field of patent law.

As used herein, the terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, cycloalkynyl or aryl group, or the total number of carbon atoms and heteroatoms in a heteroalkyl, heterocyclyl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂, CH₃CH₂CH(CH₃)— and (CH₃)₃C—. If no “a” and “b” are designated with regard to an alkyl, alkenyl, alkynyl, cycloalkyl cycloalkenyl, cycloalkynyl, aryl, heteroaryl or heteroalicyclyl group, the broadest range described in these definitions is to be assumed.

As used herein, the term “acyl” refers to residues derived from substituted or unsubstituted acids including, but not limited to, carboxylic acids, carbamic acids, carbonic acids, sulfonic acids, and phosphorous acids. Examples include aliphatic carbonyls, aromatic carbonyls, aliphatic sulfonyls, aromatic sulfinyls, aliphatic sulfinyls, aromatic phosphates, aliphatic phosphates, and the like.

As used herein, the term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In certain embodiments, a straight chain or branched chain alkyl has 50 or fewer carbon atoms in its backbone (e.g., C₁-C₅₀ for straight chain, C₁-C₅₀ for branched chain).

Moreover, the term alkyl includes both “unsubstituted alkyls” and “substituted alkyls”, the latter of which refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. An “alkylaryl” or an “arylalkyl” moiety is an alkyl substituted with an aryl (e.g., phenylmethyl (benzyl)).

As used herein, the term “alkenyl” includes unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups which include oxygen, nitrogen, sulfur or phosphorous atoms replacing one or more carbons of the hydrocarbon backbone. In certain embodiments, a straight chain or branched chain alkenyl group has 50 or fewer carbon atoms in its backbone (e.g., C₂-C₅₀ for straight chain, C₃-C₅₀ for branched chain).

Moreover, the term alkenyl includes both “unsubstituted alkenyls” and “substituted alkenyls,” the latter of which refers to alkenyl moieties having substituents replacing a hydrogen on one or more carbons of the hydrocarbon backbone. Such substituents can include, for example, alkyl groups, alkenyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other examples of alkenyl groups include, but are not limited to, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl groups. Groups containing multiple double bonds may include but are not limited to buta-1,3-dienyl, penta-1,3-dienyl or penta-1,4-dienyl groups.

As used herein, the term “alkynyl” includes unsaturated aliphatic groups analogous in length and possible substitution to the alkyls described above, but which contain at least one triple bond. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups which include oxygen, nitrogen, sulfur or phosphorous atoms replacing one or more carbons of the hydrocarbon backbone. In some embodiments, a straight chain or branched chain alkynyl group has 50 or fewer carbon atoms in its backbone (e.g., C₂-C₅₀ for straight chain, C₃-C₅₀ for branched chain).

Moreover, the term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, for example, alkyl groups, alkenyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Groups containing multiple triple bonds may include but are not limited to buta-1,3-diynyl, penta-1,3-diynyl or penta-1,4-diynyl groups.

As used herein, the terms “analog” or “derivative” are used in accordance with its plain ordinary meaning within chemistry and biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

As used herein, the terms “cycloalkyl” and “heterocycloalkyl,” by themselves or in combination with other terms, mean, unless otherwise stated, cyclic versions of “alkyl” and “heteroalkyl,” respectively. In some embodiments, a “heterocycloalkyl” is also referred as a “heterocyclic” group or moiety. Cycloalkyl and heterocycloalkyl are not aromatic. Cycloalkyl and heterocycloalkyl can be further substituted, e.g., with any of the substituents described herein.

Each of the terms (e.g., “alkyl,” “aromatic,” “heteroalkyl,” “cycloalkyl,” “heterocyclic,” etc.) includes both substituted and unsubstituted forms of the indicated radical. In that regard, whenever a group or moiety is described as being “substituted” or “optionally substituted” (or “optionally having” or “optionally comprising”) that group may be unsubstituted or substituted with one or more of the indicated substituents. Likewise, when a group is described as being “substituted or unsubstituted” if substituted, the substituent(s) may be selected from one or more of the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, cyanate, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, an ether, amino (e.g. a mono-substituted amino group or a di-substituted amino group), and protected derivatives thereof. Any of the above groups may include one or more heteroatoms, including O, N, or S. For example, where a moiety is substituted with an alkyl group, that alkyl group may comprise a heteroatom selected from O, N, or S (e.g. —(CH₂—CH₂—O—CH₂—CH₃)).

As used herein, the term “heteroatom” is meant to include boron (B), oxygen (O), nitrogen (N), sulfur (S), phosphorus (P), and silicon (Si). As noted herein, in some embodiments, a “heterocyclic ring” may comprise one or more heteroatoms. In other embodiments, an aliphatic group may comprise or be substituted by one or more heteroatoms.

As used herein, the term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen, phosphorus, and sulfur atoms may optionally be oxidized, and the nitrogen heteroatom may optionally be quaternate. The heteroatom(s) O, N, P, S, and Si may be placed at any interior position of the heteroalkyl group or at the position at which the alkyl group is attached to the remainder of the molecule. A heteroalkyl is not cyclized. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—O—CH₃, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

As used herein, the terms “couple” or “coupling” refer to the joining, bonding (e.g. covalent bonding), or linking of one molecule or atom to another molecule or atom.

As used herein, the term “leaving group” refers to any group that is the conjugate base of a strong acid. Leaving groups which are useful in the present invention include, but are not limited to, halogen, alkylsulfonyl, substituted alkylsulfonyl, arylsulfonyl, substituted arylsulfonyl, heterocyclcosulfonyl or trichloroacetimidate. In some embodiments, the leaving group is chloro, fluoro, bromo, iodo, p-(2,4-dinitroanilino)benzenesulfonyl, benzenesulfonyl, methylsulfonyl(mesylate), p-methylbenzene-sulfonyl (tosylate), p-bromobenzenesulfonyl, trifluoromethyl-sulfonyl(triflate), trichloroacetimidate, acyloxy, 2,2,2-trifluoroethanesulfonyl, imidazolesulfonyl, and 2,4,6 trichlorophenyl, with chloro being preferred.

As used herein, the term “phosphoramidite” refers to a trivalent phosphorus group typically used in oligonucleotide synthesis. Detailed descriptions of the chemistry used to form oligonucleotides by the phosphoramidite method are provided in Caruthers et al., U.S. Pat. Nos. 4,458,066 and 4,415,732; Caruthers et al., Genetic Engineering, 4:1-17 (1982); Users Manual Model 392 and 394 Polynucleotide Synthesizers, pages 6-1 through 6-22, Applied Biosystems, Part No. 901237 (1991), each of which are incorporated by reference in their entirety.

As used herein, the term “protecting group” refers to a moiety that when attached to a reactive group in a molecule masks, reduces or prevents that reactivity. A “protected” molecule has one or more reactive groups (e.g., hydroxyl, amino, thiol, etc.) protected by protecting groups. Examples of protecting groups can be found in T. W. Greene and P. G. M. Wuts, Protective Groups in Organic Synthesis, 3rd edition, John Wiley & Sons, New York, 1999, Harrison and Harrison et al. Compendium of Synthetic Organic Methods, Vols. 1-8 (John Wiley and Sons, 1971-1996), and “Protection of Nucleosides for Oligonucleotide Synthesis,” Current Protocols in Nucleic Acid Chemistry, ed. by Boyle, A. L., John Wiley & Sons, Inc., 2000, New York, N.Y., all of which are incorporated herein by reference in their entirety. Examples of hydroxyl protecting groups include, but are not limited to, benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl (BOC), isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl (Alloc), acetyl (Ac), formyl, chloroacetyl, trifluoroacetyl, methoxyacetyl, phenoxyacetyl, benzoyl (Bz), methyl, t-butyl, 2,2,2-trichloroethyl, 2-trimethylsilyl ethyl, 1,1-dimethyl-2-propenyl, 3-methyl-3-butenyl, allyl, benzyl (Bn), para-methoxybenzyldiphenylmethyl, triphenylmethyl(trityl), 4,4′-dimethoxytriphenylmethyl (DMT), substituted or unsubstituted 9-(9-phenyl)xanthenyl(pixyl), tetrahydrofuryl, methoxymethyl, methylthiomethyl, benzyloxymethyl, 2,2,2-trichloroethoxymethyl, 2-(trimethylsilyl)ethoxymethyl, methanesulfonyl, para-toluenesulfonyl, trimethylsilyl, triethylsilyl, triisopropylsilyl, and the like.

As used herein, the terms “reactive group” or “reactive functional group” refer to a functional group that are capable of chemically associating with, interacting with, hybridizing with, hydrogen bonding with, or coupling with a functional group of a different moiety. In some embodiments, a “reaction” between two reactive groups or two reactive functional groups may mean that a covalent linkage is formed between two reactive groups or two reactive functional groups; or may mean that the two reactive groups or two reactive functional groups associate with each other, interact with each other, hybridize to each other, hydrogen bond with each other, etc. In some embodiments, the “reaction” thus includes binding events, such as the binding of a hapten with an anti-hapten antibody, or a guest molecule associating with a supramolecular host molecule.

As used herein, the symbol “ [symbol] ” refers to a location in which a moiety is bonded to another moiety.

As used herein, the abbreviations “DMT” or “DMTr” (used interchangeably) refer to 4,4′-dimethoxytrityl, which has the chemical structure:

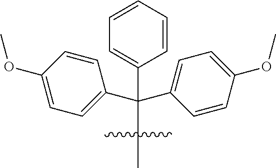

### Overview

The present disclosure relates to a process for the regiochemically and enantiomerically controlled synthesis of phosphoramidite-containing monomers, and to intermediate products of this process. In some embodiments, the phosphoramidite-containing monomers are derived from polyols, such as glycerol, mannitol, erythulose, dihydroxypropanal, or dihydroxypropanoate or derivatives or analogs thereof. In some embodiments, the phosphoramidite-containing monomers are at least 97% regioisomerically and/or enantiomerically pure. In other embodiments, the phosphoramidite-containing monomers are at least 98% regioisomerically and/or enantiomerically pure. In yet other embodiments, the phosphoramidite-containing monomers are at least 99% regioisomerically and/or enantiomerically pure. In some embodiments, oligomers, polymers, or copolymers, such as oligomers, polymers, or copolymers incorporated in XNTP molecules, may be derived from one or more phosphoramidite-containing monomers (see PCT Publication No. WO/2020/236526, which provides examples of phosphoramidite-containing monomers, the disclosure of which is hereby incorporated by reference herein in its entirety).

In some embodiments, the intermediate products described herein include a protecting group which includes a methylene group (—CH₂—) between a protected alcohol and a residual portion of the protecting group moiety. By way of example, —CH₂—N₃ may be employed as a protecting group, where the —CH₂—N₃ moiety includes a methylene group between the protected alcohol and the azide. Non-limiting examples of other protecting groups that meet this criteria are described herein.

In some embodiments, the protecting group is selected such that its incorporation and subsequent removal provides isomerization free products, i.e., products that are least 97% regioisomerically and/or enantiomerically pure, at least 98% regioisomerically and/or enantiomerically pure, or at least 99% regioisomerically and/or enantiomerically pure. In some embodiments, the protecting group is selected such that it does not migrate in any subsequent downstream reactions or transformations. In some embodiments, the protecting group is selected such that in the course of protection, transformation, and deprotection reactions, the protecting group and reagents utilized do not cause any racemization of other carbon atoms in any starting materials or subsequently synthesized intermediates, e.g., racemization of secondary carbon atoms in polyols, such as glycerol, mannitol, erythrulose, dihydroxypropanal, or dihydroxypropanoate starting materials.

In some embodiments, the protecting group is selected from one which is stable in subsequent synthetic steps. For instance, the protecting group is selected from one that is (i) predominantly stable under mild to moderately acidic conditions; and/or (ii) stable under basic and alkylating conditions. In some embodiments, the protecting group is selected such that it permits moderate to high yields in subsequent substitution reactions with long and/or bulky side chains, such as polyalkylene oxide (e.g., polyalkylene oxide linkers), alkyl, acyl, alkenyl, or alkynyl side chains or derivatives or analogs thereof. In some embodiments, the protecting group is selected such that it is entirely removable without pronounced decomposition or transformation of other functional groups in the protected molecule, such as base-labile esters, acid-labile trityl-protected alcohols, or oxidation-sensitive polyalkylene oxide-linkers (e.g., polyalkylene oxide linkers).

Suitable protecting groups that meet one or more of the aforementioned criteria are described in Wuts, P. G. M., & Greene, T. W. (2007). Greene's protective groups in organic synthesis, fourth edition (4th ed.). Wiley-Interscience; T. W. Greene and P. G. M. Wuts, “Protective Groups in Organic Synthesis,” 2nd ed., John Wiley & Sons, Inc., New York, N.Y., 1991, chapter 5; E. Haslam, “Protective Groups in Organic Chemistry,” J. G. W. McOmie, Ed., Plenum Press, New York, N.Y., 1973, Chapter 5, and T. W. Greene, “Protective Groups in Organic Synthesis,” John Wiley and Sons, New York, NY, 1981, Chapter 5, the disclosures of which are hereby incorporated by reference herein in their entireties.

### Intermediates for the Synthesis of Phosphoramidite-Containing Monomers

One aspect of the present disclosure are intermediates useful for the synthesis of phosphoramidite-containing monomers. Examples of phosphoramidite-containing monomers are set forth in Formulas (VIIIA) to (VIIID). In some embodiments, the intermediates of phosphoramidite-containing monomers disclosed herein are substantially regioisomerically and/or enantiomerically pure, i.e., they are at least 97% regioisomerically and/or enantiomerically pure, at least 98% regioisomerically and/or enantiomerically pure, or at least 99% regioisomerically and/or enantiomerically pure.

In some embodiments, the intermediates in the synthesis of phosphoramidite-containing monomers have the structure of any one of Formulas (IA) and (IB):

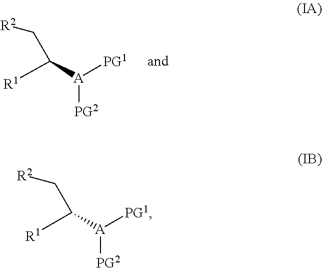

wherein
R¹ is —OH, —O—R^{w}—Z, where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, or —CH₂-Het, where “Het” is a substituted or unsubstituted 5-membered heterocyclic moiety;
R² is —OH, —O-trityl or a derivative or analog thereof, —O-9-phenylthioxanthyl (pixyl) or a derivative or analog thereof, —O-2-(2-nitrophenyl)prop-1-oxycarbonyl (“NPPOC”), —O-(2-nitrobenzyl) or a derivative or analog thereof, —O-(1-(2-nitrophenyl)ethyl) or a derivative or analog thereof, —O-1-(2-fluorophenyl)-4-methoxypiperidin-4-yl, —O-[(chloro-4-methyl)phenyl]-4′-methoxypiperidin-4-yl, tetrahydropyranyl ether, ethoxyethyl ether, methallyl ether, prenyl ether, or methoxymethyl ether;
A is CH;
PG² is H;
PG¹ is

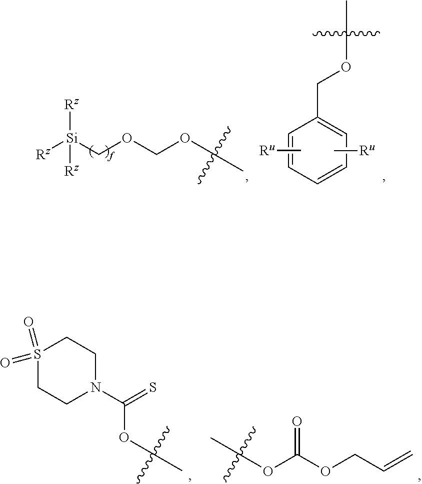

—O—CH₂—S—CH₃, —O—CH₂—N₃, or —O—CH₂—CH═CH₂; or
PG¹-A-PG² taken together form C(O)H, C(O)OMe, —C(O)OT,

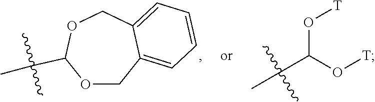

T is a C₁-C₆ branched or unbranched alkyl group;
f is 0 or 2;
each R^{u} is independently H, —O—C₁-C₄ alkyl or —C₁-C₄ alkyl; and
each R^{z} is independently a branched or unbranched C₁-C₆ alkyl group;

provided that when PG¹-A-PG² together form C(O)H, C(O)OMe, or

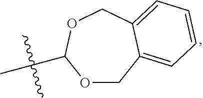 [SMILES: *C1OCc2ccccc2CO1]

R¹ and R² are both not —OH.

In some embodiments, PG¹-A-PG² is

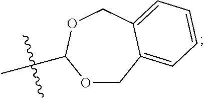 [SMILES: *C1OCc2ccccc2CO1]

and R² is —O-trityl or a derivative or analog thereof. In other embodiments, PG¹-A-PG² is

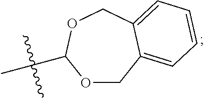 [SMILES: *C1OCc2ccccc2CO1]

R² is —O-trityl or a derivative or analog thereof; and R¹ is —OH. In some embodiments, PG¹-A-PG² is

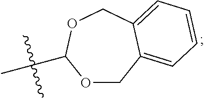 [SMILES: *C1OCc2ccccc2CO1]

and R² is —O-pixyl or a derivative or analog thereof. In other embodiments, PG¹-A-PG² is

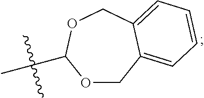 [SMILES: *C1OCc2ccccc2CO1]

R² is —O-pixyl or a derivative or analog thereof; and R¹ is —OH.

In some embodiments, PG¹ is

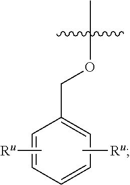

and R² is —O-trityl or a derivative or analog thereof. In some embodiments, PG¹ is

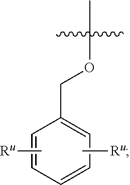

R² is —O-trityl or a derivative or analog thereof; and R¹ is —OH.

In some embodiments, PG¹ is

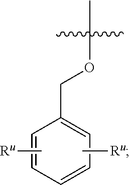

and R² is —O-pixyl or a derivative or analog thereof. In some embodiments, PG¹ is

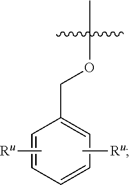

R² is —O-pixyl or a derivative or analog thereof; and R¹ is —OH.

In some embodiments, PG¹ is

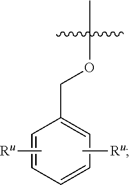

R² is —O-trityl or a derivative or analog thereof; and where PG¹ includes one R^{u} group, wherein the R^{u} group is —CH₃. In some embodiments, PG¹ is

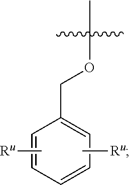

R² is —O-trityl or a derivative or analog thereof; R¹ is —OH; and where PG¹ includes one R^{u} group, wherein the R^{u} group is —CH₃.

In some embodiments, PG¹ is

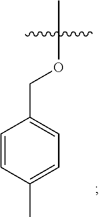 [SMILES: *OCc1ccc(C)cc1]

and R² is —O-trityl or a derivative or analog thereof. In some embodiments, PG¹ is

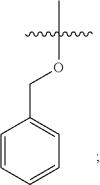 [SMILES: *OCc1ccccc1]

R² is —O-trityl or a derivative or analog thereof; and R¹ is —OH.

In some embodiments, PG¹ is

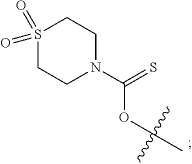 [SMILES: *OC(=S)N1CCS(=O)(=O)CC1]

and R² is —O-trityl or a derivative or analog thereof. In some embodiments, PG¹ is

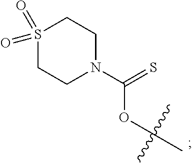 [SMILES: *OC(=S)N1CCS(=O)(=O)CC1]

R² is —O-trityl or a derivative or analog thereof; and R¹ is —OH.

In some embodiments, PG¹ is

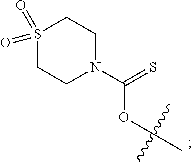 [SMILES: *OC(=S)N1CCS(=O)(=O)CC1]

and R² is —O-pixyl or a derivative or analog thereof. In some embodiments, PG¹ is

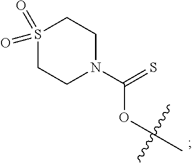 [SMILES: *OC(=S)N1CCS(=O)(=O)CC1]

R² is —O-pixyl or a derivative or analog thereof; and R¹ is —OH.

In some embodiments, PG¹ is —O—CH₂—N₃; and R² is —O-trityl or a derivative or analog thereof. In some embodiments, PG1 is —O—CH₂—N₃; R² is —O-trityl or a derivative or analog thereof; and R¹ is —OH. In some embodiments, PG¹ is —O—CH₂—N₃; and R² is —O-pixyl or a derivative or analog thereof. In some embodiments, PG¹ is —O—CH₂—N₃; R² is —O-pixyl or a derivative or analog thereof; and R¹ is —OH.

In some embodiments, PG¹ is —O—CH₂—S—CH₃, and R² is —O-trityl or a derivative or analog thereof. In some embodiments, PG1 is —O—CH₂—S—CH₃, R² is —O-trityl or a derivative or analog thereof; and R¹ is —OH. In some embodiments, PG¹ is —O—CH₂—S—CH₃, and R² is —O— pixyl or a derivative or analog thereof. In some embodiments, PG¹ is —O—CH₂—S—CH₃, R² is —O— pixyl or a derivative or analog thereof; and R¹ is —OH.

In some embodiments, PG¹ is —O—CH₂—CH═CH₂; and R² is —O-trityl or a derivative or analog thereof. In some embodiments, PG¹ is —O—CH₂—CH═CH₂; R² is —O-trityl or a derivative or analog thereof; and R¹ is —OH. In some embodiments, PG¹ is-O—CH₂—CH═CH₂; and R² is —O-pixyl or a derivative or analog thereof. In some embodiments, PG¹ is —O—CH₂—CH═CH₂; R² is —O-pixyl or a derivative or analog thereof; and R¹ is —OH.

In some embodiments, PG¹-A-PG² is C(O)H or C(O)OMe; and R² is —O-trityl or a derivative or analog thereof. In some embodiments, PG¹-A-PG² is C(O)H or C(O)OMe; R² is —O-trityl or a derivative or analog thereof; and R¹ is —OH. In some embodiments, PG¹-A-PG² is C(O)H or C(O)OMe; and R² is —O-pixyl or a derivative or analog thereof. In some embodiments, PG¹-A-PG² is C(O)H or C(O)OMe; R² is —O-pixyl or a derivative or analog thereof; and R¹ is —OH.

In some embodiments, PG¹-A-PG² is

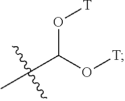

and R² is —O-trityl or a derivative or analog thereof. In some embodiments, PG¹-A-PG² is

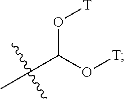

R² is —O-trityl or a derivative or analog thereof; and R¹ is —OH. In some embodiments, PG¹-A-PG² is

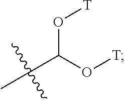

and R² is —O-pixyl or a derivative or analog thereof. In some embodiments, PG¹-A-PG² is

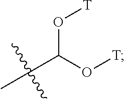

R² is —O-pixyl or a derivative or analog thereof; and R¹ is —OH.

In some embodiments, PG¹ is

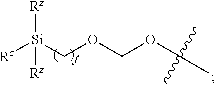

and R² is —O-trityl or a derivative or analog thereof. In some embodiments, PG¹ is

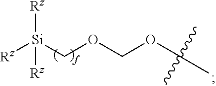

R² is —O-trityl or a derivative or analog thereof; and R¹ is —OH.

In some embodiments, PG¹ is

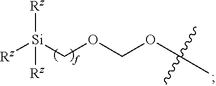

and R² is —O-pixyl or a derivative or analog thereof. In some embodiments, PG¹ is

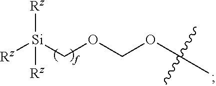

R² is —O-pixyl or a derivative or analog thereof; and R¹ is —OH.

In some embodiments, PG¹ is

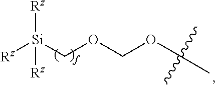

and f is 0. In some embodiments, PG¹ is

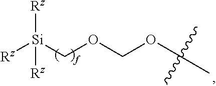

f is 0; and R² is —O-trityl. In some embodiments, PG¹ is

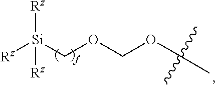

f is 0; R² is —O-trityl; and R¹ is —OH.

In other embodiments, PG¹ is

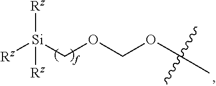

and f is 2. In some embodiments, PG¹ is

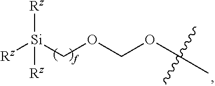

f is 2; and R² is —O-trityl. In some embodiments, PG¹ is

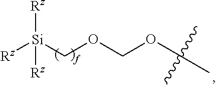

f is 2; R² is —O-trityl; and R¹ is —OH. In some embodiments, PG¹ is

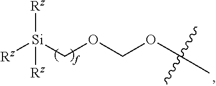

f is 0; and R^{z} is a branched or unbranched C₁-C₆ group.

In other embodiments, PG¹ is

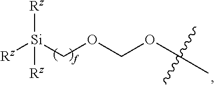

f is 2; and R^{z} is a branched or unbranched C₁-C₆ group.

In some embodiments, PG¹ is

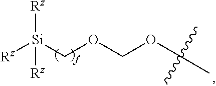

f is 0; and R^{z} is a branched or unbranched C₁-C₆ group.

In some embodiments, PG¹ is

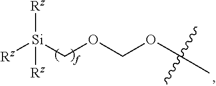

f is 0; and R^{z} is a branched or unbranched C₁-C₃ group.

In other embodiments, PG¹ is

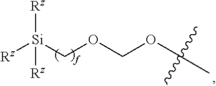

f is 2; and R^{z} is a branched or unbranched C₁-C₆ group.

In other embodiments, PG¹ is

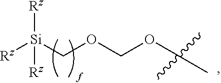

f is 2; and R^{z} is a branched or unbranched C₁-C₃ group.

In some embodiments, R² has the structure:

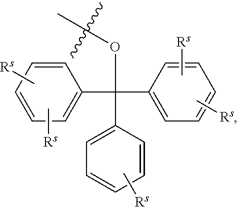

where each R^{s} is independently H, —O—C₁-C₄ alkyl or —C₁-C₄ alkyl. In some embodiments, each R^{s} is independently selected from H, —O—C₁-C₃ alkyl or —C₁-C₃ alkyl. In some embodiments, each R^{s} is independently selected from H, —O—C₁-C₂ alkyl or —C₁-C₂ alkyl. In some embodiments, each R^{s} is independently selected from H, —O—CH₃ or —CH₃.

In some embodiments, R² has the structure:

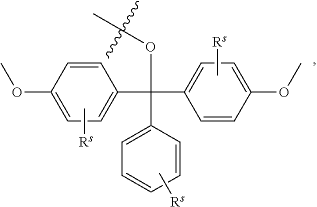

where each R^{s} is independently H, —O—C₁-C₄ alkyl or —C₁-C₄ alkyl. In some embodiments, each R^{s} is independently selected from H, —O—C₁-C₃ alkyl or —C₁-C₃ alkyl. In some embodiments, each R^{s} is independently selected from H, —O—C₁-C₂ alkyl or —C₁-C₂ alkyl. In some embodiments, each R^{s} is independently selected from H, —O—CH₃ or —CH₃.

In some embodiments, R² has the structure:

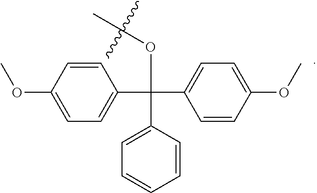

In some embodiments, R² is —O-pixyl or a derivative or analog thereof. Non-limiting examples of derivatives or analogs of pixyl moieties are described in United States Patent Application 2007/0276139, the disclosure of which is hereby incorporated by reference herein in its entirety.

In some embodiments, R^{w} is a substituted or unsubstituted, branched or unbranched, saturated or unsaturated alkyl moiety having between 2 and 80 carbon atoms, and which optionally includes one or more oxygen heteroatoms. In some embodiments, R^{w} is a substituted or unsubstituted, branched or unbranched, saturated or unsaturated alkyl moiety having between 2 and 60 carbon atoms, and which optionally includes one or more oxygen heteroatoms. In some embodiments, R^{w} is a substituted or unsubstituted, branched or unbranched, saturated or unsaturated alkyl moiety having between 4 and 48 carbon atoms, and which optionally includes one or more oxygen heteroatoms. In some embodiments, R^{w} is a substituted or unsubstituted, branched or unbranched, saturated or unsaturated alkyl moiety having between 4 and 24 carbon atoms, and which optionally includes one or more oxygen heteroatoms. In some embodiments, R^{w} is a substituted or unsubstituted, branched or unbranched, saturated or unsaturated alkyl moiety having between 4 and 12 carbon atoms, and which optionally includes one or more oxygen heteroatoms.

In some embodiments, R^{w} is an unsubstituted alkyl moiety having between 2 and 80 carbon atoms, and which optionally includes one or more oxygen heteroatoms. In some embodiments, R^{w} is an unsubstituted alkyl moiety having between 2 and 60 carbon atoms, and which optionally includes one or more oxygen heteroatoms. In some embodiments, R^{w} is an unsubstituted alkyl moiety having between 4 and 48 carbon atoms, and which optionally includes one or more oxygen heteroatoms. In some embodiments, R^{w} is an unsubstituted alkyl moiety having between 4 and 24 carbon atoms, and which optionally includes one or more oxygen heteroatoms. In some embodiments, R^{w} is an unsubstituted alkyl moiety having between 4 and 12 carbon atoms, and which optionally includes one or more oxygen heteroatoms.

In some embodiments, R^{w} includes at least one polyethylene glycol group (“PEG”), at least two PEG groups, at least three PEG groups, at least four PEG groups, at least 8 PEG groups, at least 12 PEG groups, or at least 24 PEG groups.

In some embodiments, R^{w} includes at least one PEG group, at least two PEG groups, at least three PEG groups, at least four PEG groups, at least 8 PEG groups, at least 12 PEG groups, or at least 24 PEG groups; and where Z is an alkyl group, e.g., a C₁-C₄ alkyl group, methyl, or ethyl.

In some embodiments, R^{w} includes at least one PEG group, at least two PEG groups, at least three PEG groups, at least four PEG groups, at least 8 PEG groups, at least 12 PEG groups, or at least 24 PEG groups; and where Z is an alkenyl group, e.g., —CH═CH₂, or —CH₂—CH═CH₂.

In some embodiments, R^{w} includes at least one PEG group, at least two PEG groups, at least three PEG groups, at least four PEG groups, at least 8 PEG groups, at least 12 PEG groups, or at least 24 PEG groups; and where Z is an alkynyl group, e.g.,

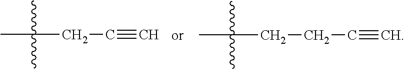

In some embodiments, R^{w} includes at least one PEG group, at least two PEG groups, at least three PEG groups, at least four PEG groups, at least 8 PEG groups, at least 12 PEG groups, or at least 24 PEG groups; and where Z is an acyl group.

In some embodiments, R^{w} includes at least one PEG group, at least two PEG groups, at least three PEG groups, at least four PEG groups, at least 8 PEG groups, at least 12 PEG groups, or at least 24 PEG groups; and where Z includes a substituted heterocyclic moiety. In some embodiments, the heterocyclic moiety is substituted with an alkylaryl group. In some embodiments, the substituted heterocyclic moiety includes a triazole. In some embodiments, the triazole is substituted with an alkylaryl group. In some embodiments, the triazole is substituted with —CH₂—CH₂—O-Ph. In some embodiments, the triazole is substituted with —CH₂—CH₂—O-Bz.

In some embodiments, the “click functional group” is selected from DBCO, TCO, maleimide, —N₃, tetrazine, thiol, 1,3-nitrone, hydrazine, and hydroxylamine.

In some embodiments, the compounds of Formulas (IA) and (IB) are regioisomerically and/or enantiomerically pure, e.g., at least 97%, at least 98%, at least 99% regioisomerically and/or enantiomerically pure.

In some embodiments, the intermediates in the synthesis of phosphoramidite-containing monomers have the structure of any one of Formulas (IIA) and (IIB):

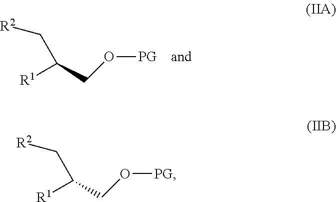

wherein
PG is

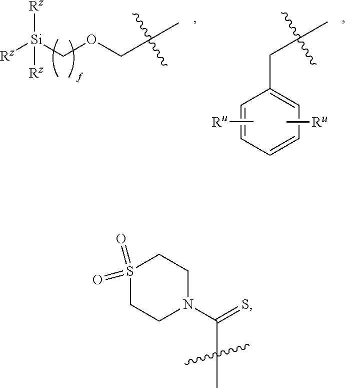

—CH₂—S—CH₃, —CH₂—N₃, or —CH₂—CH═CH₂;

R¹ is —OH, —O—R^{w}—Z, where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, or —CH₂-Het, where “Het” is a substituted or unsubstituted 5-membered heterocyclic moiety;
R² is —OH, —O-trityl or a derivative or analog thereof, —O-9-phenylthioxanthyl (pixyl) or a derivative or analog thereof, —O-2-(2-nitrophenyl)prop-1-oxycarbonyl (“NPPOC”), —O-(2-nitrobenzyl) or a derivative or analog thereof, —O-(1-(2-nitrophenyl)ethyl) or a derivative or analog thereof, tetrahydropyranyl ether, ethoxyethyl ether, methallyl ether, prenyl ether, or methoxymethyl ether;
or where or where R¹ and R² together form a substituted or unsubstituted 5-membered ring;
f is 0 or 2;
each R^{u} is independently H, —O—C₁-C₄ alkyl or —C₁-C₄ alkyl; and
each R^{z} is independently a branched or unbranched C₁-C₆ alkyl group.

In some embodiments, the “click functional group” is selected from DBCO, TCO, maleimide, —N₃, tetrazine, thiol, 1,3-nitrone, hydrazine, and hydroxylamine.

In some embodiments, PG is

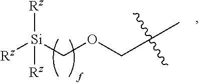

and f is 0. In other embodiments, PG is

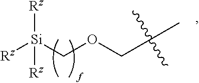

and f is 2.

In some embodiments, PG is

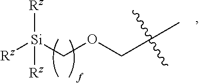

f is 0, and R^{z} is a branched or unbranched C₁-C₃ group. In other embodiments, PG is

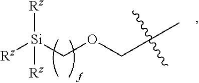

f is 2, and R^{z} is a branched or unbranched C₁-C₆ group. In other embodiments, PG is

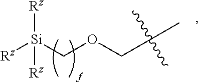

f is 2, and R^{z} is a branched or unbranched C₁-C₃ group.

In some embodiments, PG is

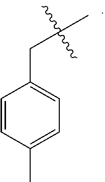 [SMILES: *Cc1ccc(C)cc1]

In some embodiments, PG is

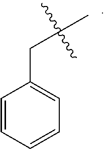 [SMILES: *Cc1ccccc1]

In some embodiments, R² has the structure:

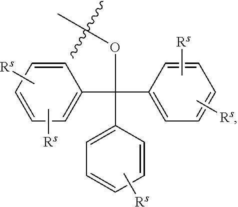

where each R^{s} is independently H, —O—C₁-C₄ alkyl or —C₁-C₄ alkyl. In some embodiments, each R^{s} is independently selected from H, —O—C₁-C₃ o alkyl r —C₁-C₃ alkyl. In some embodiments, each R^{s} is independently selected from H, —O—C₁-C₂ alkyl or —C₁-C₂ alkyl. In some embodiments, each R^{s} is independently selected from H, —O—CH₃ or —CH₃.

In some embodiments, R² has the structure:

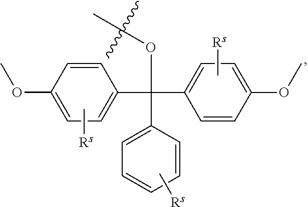

where each R^{s} is independently H, —O—C₁-C₄ alkyl or —C₁-C₄ alkyl. In some embodiments, each R^{s} is independently selected from H, —O—C₁-C₃ alkyl or —C₁-C₃ alkyl. In some embodiments, each R^{s} is independently selected from H, —O—C₁-C₂ alkyl or —C₁-C₂ alkyl. In some embodiments, each R^{s} is independently selected from H, —O—CH₃ or —CH₃.

In some embodiments, R² has the structure:

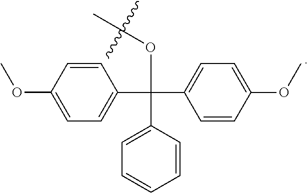

In some embodiments, R² is pixyl or a derivative or along thereof. Non-limiting examples of derivatives or analogs of pixyl are described in United States Patent Application 2007/0276139 the disclosure of which is hereby incorporated by reference herein in its entirety.

In some embodiments, R^{w} is a substituted or unsubstituted, branched or unbranched, saturated or unsaturated alkyl moiety having between 2 and 80 carbon atoms, and which optionally includes one or more oxygen heteroatoms. In some embodiments, R^{w} is a substituted or unsubstituted, branched or unbranched, saturated or unsaturated alkyl moiety having between 2 and 60 carbon atoms, and which optionally includes one or more oxygen heteroatoms. In some embodiments, R^{w} is a substituted or unsubstituted, branched or unbranched, saturated or unsaturated alkyl moiety having between 4 and 48 carbon atoms, and which optionally includes one or more oxygen heteroatoms. In some embodiments, R^{w} is a substituted or unsubstituted, branched or unbranched, saturated or unsaturated alkyl moiety having between 4 and 24 carbon atoms, and which optionally includes one or more oxygen heteroatoms. In some embodiments, R^{w} is a substituted or unsubstituted, branched or unbranched, saturated or unsaturated alkyl moiety having between 4 and 12 carbon atoms, and which optionally includes one or more oxygen heteroatoms.

In some embodiments, R^{w} is an unsubstituted alkyl moiety having between 2 and 80 carbon atoms, and which optionally includes one or more oxygen heteroatoms. In some embodiments, R^{w} is an unsubstituted alkyl moiety having between 2 and 60 carbon atoms, and which optionally includes one or more oxygen heteroatoms. In some embodiments, R^{w} is an unsubstituted alkyl moiety having between 4 and 48 carbon atoms, and which optionally includes one or more oxygen heteroatoms. In some embodiments, R^{w} is an unsubstituted alkyl moiety having between 4 and 24 carbon atoms, and which optionally includes one or more oxygen heteroatoms. In some embodiments, R^{w} is an unsubstituted alkyl moiety having between 4 and 12 carbon atoms, and which optionally includes one or more oxygen heteroatoms.

In some embodiments, R^{w} includes at least one PEG group, at least two PEG groups, at least three PEG groups, at least four PEG groups, at least 8 PEG groups, at least 12 PEG groups, or at least 24 PEG groups.

In some embodiments, R^{w} includes at least one PEG group, at least two PEG groups, at least three PEG groups, at least four PEG groups, at least 8 PEG groups, at least 12 PEG groups, or at least 24 PEG groups; and where Z is an alkenyl group, e.g., —CH═CH₂, or —CH₂—CH═CH₂.

In some embodiments, R^{w} includes at least one PEG group, at least two PEG groups, at least three PEG groups, at least four PEG groups, at least 8 PEG groups, at least 12 PEG groups, or at least 24 PEG groups; and where Z is an alkynyl group, e.g.,

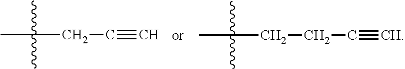

In some embodiments, R^{w} includes at least one PEG group, at least two PEG groups, at least three PEG groups, at least four PEG groups, at least 8 PEG groups, at least 12 PEG groups, or at least 24 PEG groups; and where Z is an acyl group.

In some embodiments, R^{w} includes at least one PEG group, at least two PEG groups, at least three PEG groups, at least four PEG groups, at least 8 PEG groups, at least 12 PEG groups, or at least 24 PEG groups; and where Z includes a substituted heterocyclic moiety. In some embodiments, the heterocyclic moiety is substituted with an alkylaryl group. In some embodiments, the substituted heterocyclic moiety includes a triazole. In some embodiments, the triazole is substituted with an alkylaryl group. In some embodiments, the triazole is substituted with —CH₂—CH₂—O-Ph. In some embodiments, the triazole is substituted with —CH₂—CH₂—O-Bz.

In some embodiments, the intermediates of Formulas (HA) and (IIB) have Formulas (IIIA) and (IIIB), respectively:

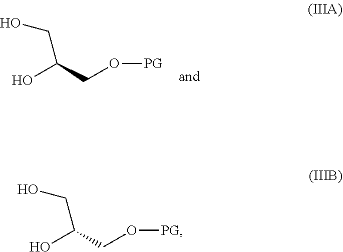

wherein PG is as defined above.

Non-limiting examples of compounds having any one of Formulas (IIIA) or (IIIB) include:

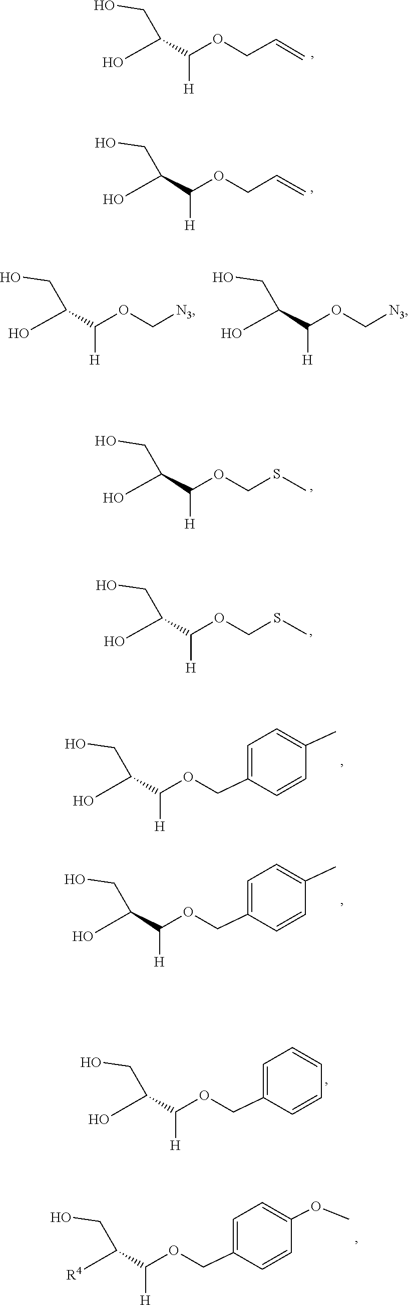

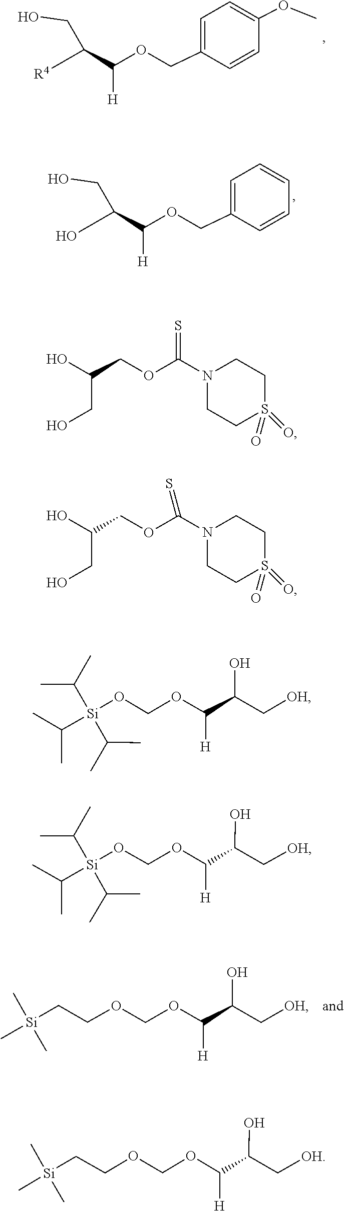

In some embodiments, the intermediates of Formulas (IIA) and (IIB) have Formulas (IVA) and (IVB), respectively:

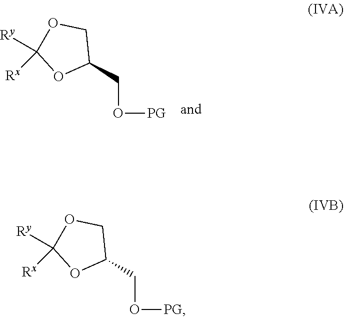

wherein PG is as defined above, and

wherein R^{x} and R are independently a C₁-C₄ branched or unbranched alkyl group.

In some embodiments, R^{x} and R^{y} are each independently a C₁-C₂ alkyl group. In some embodiments, R^{x} and R^{y} are both methyl.

In some embodiments, the intermediates of Formulas (HA) and (IIB) have Formulas (VA) and (VB), respectively:

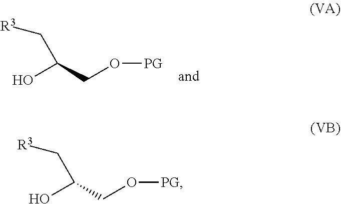

where PG is as defined above; and where R³ is —O-trityl or a derivative or analog thereof, or —O-pixyl or a derivative or analog thereof.

In some embodiments, R³ has the structure:

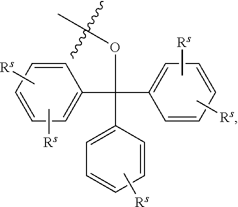

where each R^{s} is independently H, —O—C₁-C₄ alkyl or —C₁-C₄ alkyl. In some embodiments, each R^{s} is independently selected from H, —O—C₁-C₃ alkyl or —C₁-C₃ alkyl. In some embodiments, each R^{s} is independently selected from H, —O—C₁-C₂ alkyl or —C₁-C₂ alkyl. In some embodiments, each R^{s} is independently selected from H, —O—CH₃ or —CH₃.

In some embodiments, R³ has the structure:

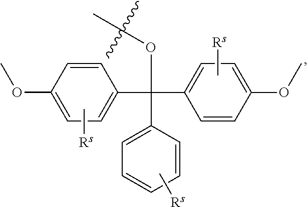

where each R^{s} is independently H, —O—C₁-C₄ alkyl or —C₁-C₄ alkyl. In some embodiments, each R^{s} is independently selected from H, —O—C₁-C₃ alkyl or —C₁-C₃ alkyl. In some embodiments, each R^{s} is independently selected from H, —O—C₁-C₂ alkyl or —C₁-C₂ alkyl. In some embodiments, each R^{s} is independently selected from H, —O—CH₃ or —CH₃.

In some embodiments, R³ has the structure:

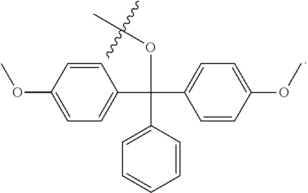

Non-limiting examples of compounds having any one of Formulas (VA) or (VB) include:

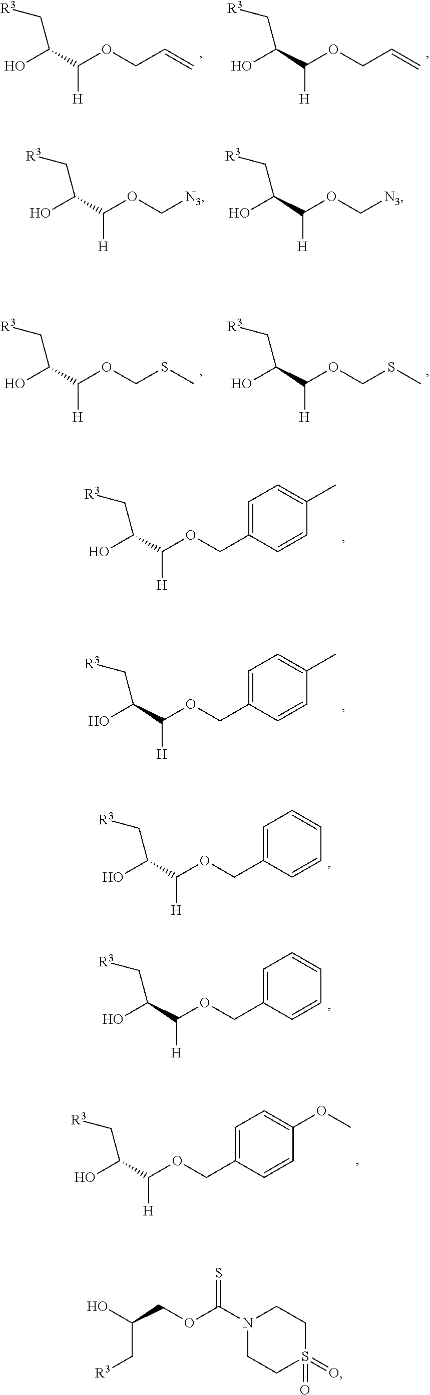

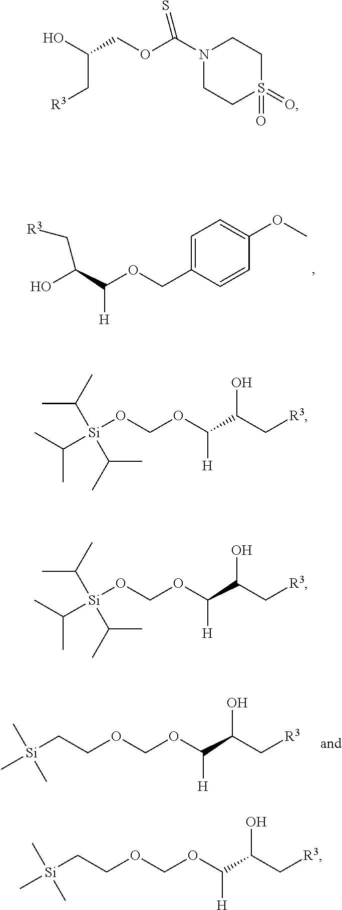

where R³ is 4,4′-dimethoxytrityl ether, 4-methoxytrityl ether, or —O-(9-phenylthioxanthyl).

In some embodiments, the intermediates of Formulas (HA) and (IIB) have Formulas (VIA) and (VIB), respectively:

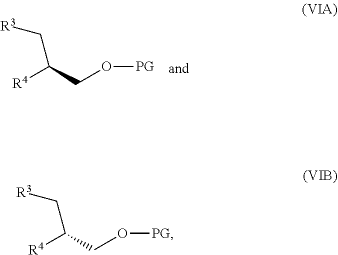

wherein PG is as defined above; R³ is —O-trityl or a derivative or analog thereof, or —O-pixyl or a derivative or analog thereof; and R⁴ has any one of Formulas (XIIIA) and (XIIIB):

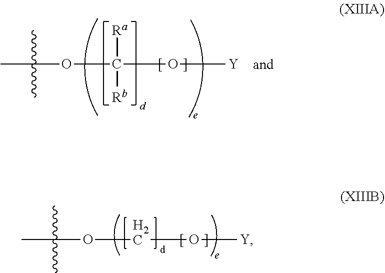

where
R^{a} and R^{b} are independently H or a C₁-C₄ alkyl;
Y is alkyl, alkenyl, alkynyl, acyl, —CH₂-alkynyl, a “click functional group,” -Het, or —CH₂-Het, where “Het” is a substituted or unsubstituted 5-membered heterocyclic moiety;
d is an integer ranging from between 1 to about 10; and
e is an integer ranging from between 1 to about 36.

In some embodiments, d is an integer ranging from 1 to about 8. In other embodiments, d is an integer ranging from 1 to about 6. In yet other embodiments, d is an integer ranging from 1 to about 2. In further embodiments, d is 2. In yet even further embodiments, d is 1.

In some embodiments, e is an integer ranging from 1 to about 24. In other embodiments, e is an integer ranging from 1 to about 20. In yet other embodiments, e is an integer ranging from 1 to about 16. In further embodiments, e is an integer ranging from 1 to about 12. In even further embodiments, e is an integer ranging from 1 to about 8. In yet further embodiments, e is an integer ranging from 1 to about 4. In yet even further embodiments, e is 12. In yet even further embodiments, e is 8. In yet even further embodiments, e is 4. In yet even further embodiments, e is 2.

In some embodiments, d is 1 and e is an integer ranging from 1 to 24. In some embodiments, d is 1 and e is an integer ranging from 1 to 12. In some embodiments, d is 1 and e is an integer ranging from 1 to 8. In some embodiments, d is 1 and e is an integer ranging from 1 to 4.

In some embodiments, Y is a substituted with an alkylaryl group. In some embodiments, the substituted heterocyclic moiety includes a triazole. In some embodiments, the triazole is substituted with an alkylaryl group. In some embodiments, the triazole is substituted with —CH₂—CH₂—O-Ph. In some embodiments, the triazole is substituted with —CH₂—CH₂—O-Bz.

In some embodiments, d is 1, e is an integer ranging from 1 to 24, and Y is alkynyl or —CH₂-alkynyl. In some embodiments, d is 1, e is an integer ranging from 1 to 12, and Y is alkynyl or —CH₂-alkynyl. In some embodiments, d is 1, e is an integer ranging from 1 to 8, and Y is alkynyl or —CH₂-alkynyl. In some embodiments, d is 1, e is an integer ranging from 1 to 4, and Y is alkynyl or —CH₂-alkynyl.

In some embodiments, d is 2, e is an integer ranging from 1 to 24, and Y is alkynyl or —CH₂-alkynyl. In some embodiments, d is 2, e is an integer ranging from 1 to 12, and Y is alkynyl or —CH₂-alkynyl. In some embodiments, d is 2, e is an integer ranging from 1 to 8, and Y is alkynyl or —CH₂-alkynyl. In some embodiments, d is 2, e is an integer ranging from 1 to 4, and Y is alkynyl or —CH₂-alkynyl.

In some embodiments, d is 1, e is an integer ranging from 1 to 24, and Y is alkenyl. In some embodiments, d is 1, e is an integer ranging from 1 to 12, and Y is alkenyl. In some embodiments, d is 1, e is an integer ranging from 1 to 8, and Y is alkenyl. In some embodiments, d is 1, e is an integer ranging from 1 to 4, and Y is alkenyl.

In some embodiments, d is 1, e is an integer ranging from 1 to 24, and Y is alkyl. In some embodiments, d is 1, e is an integer ranging from 1 to 12, and Y is alkyl. In some embodiments, d is 1, e is an integer ranging from 1 to 8, and Y is alkyl. In some embodiments, d is 1, e is an integer ranging from 1 to 4, and Y is alkyl.

In some embodiments, d is 1, e is an integer ranging from 1 to 24, and Y is a substituted 5-membered heterocyclic moiety. In some embodiments, d is 1, e is an integer ranging from 1 to 12, and Y is a substituted 5-membered heterocyclic moiety. In some embodiments, d is 1, e is an integer ranging from 1 to 8, and Y is a substituted 5-membered heterocyclic moiety. In some embodiments, d is 1, e is an integer ranging from 1 to 4, and Y is a substituted 5-membered heterocyclic moiety.

In some embodiments, R³ has the structure:

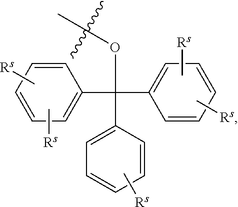

where each R^{s} is independently H, —O—C₁-C₄ alkyl or —C₁-C₄ alkyl. In some embodiments, each R^{s} is independently selected from H, —O—C₁-C₃ alkyl or —C₁-C₃ alkyl. In some embodiments, each R^{s} is independently selected from H, —O—C₁-C₂ alkyl or —C₁-C₂ alkyl. In some embodiments, each R^{s} is independently selected from H, —O—CH₃ or —CH₃.

In some embodiments, R³ has the structure:

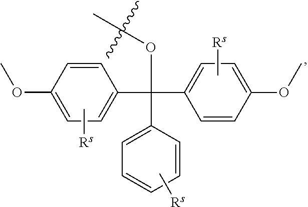

where each R^{s} is independently H, —O—C₁-C₄ alkyl or —C₁-C₄ alkyl. In some embodiments, each R^{s} is independently selected from H, —O—C₁-C₃ alkyl or —C₁-C₃ alkyl. In some embodiments, each R^{s} is independently selected from H, —O—C₁-C₂ alkyl or —C₁-C₂ alkyl. In some embodiments, each R^{s} is independently selected from H, —O—CH₃ or —CH₃.

In some embodiments, R³ has the structure:

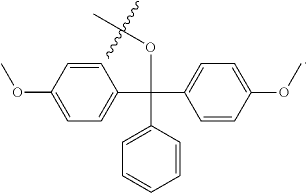

Non-limiting examples of compounds having any one of Formulas (VIA) or (VIB) include:

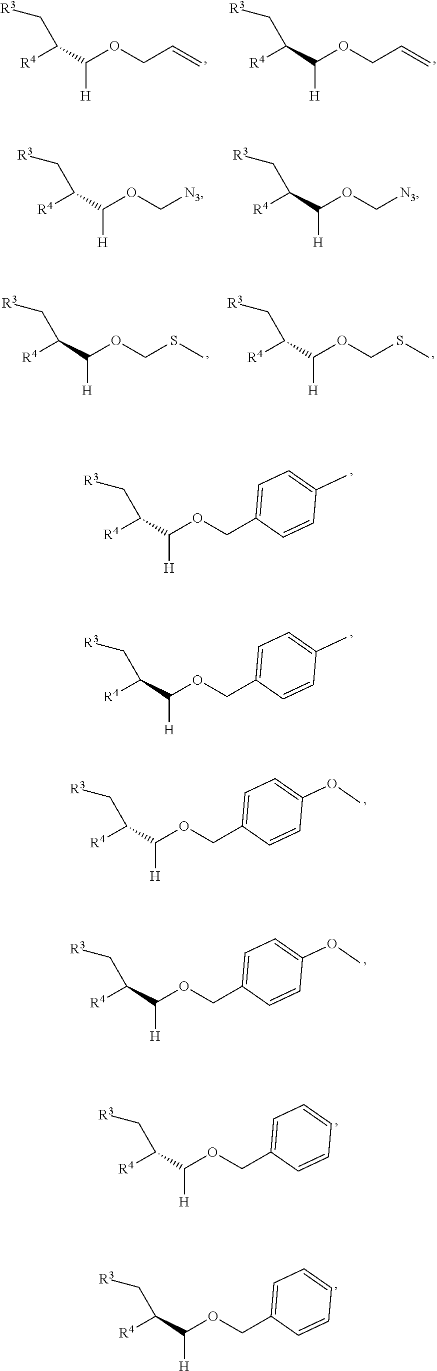

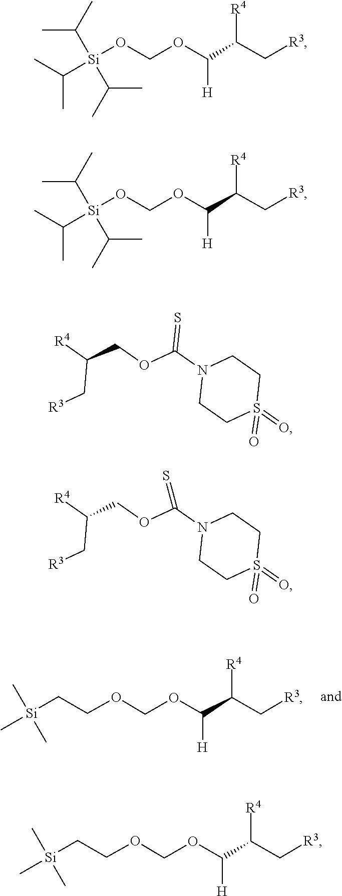

where R³ is 4,4′-dimethoxytrityl ether, 4-methoxytrityl ether, or —O-(9-phenylthioxanthyl); R⁴ is —O-PEG₂-Y, —O-PEG₃-Y, —O-PEG₄-Y, —O-PEG₈-Y, —O-PEG₁₂-Y, or —O-PEG₂₄-Y; and Y is methyl, —CH═CH₂,

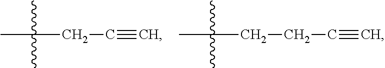

or a substituted or unsubstituted 5-membered heterocyclic moiety.

In some embodiments, the intermediates of Formulas (IIA) and (IIB) have Formulas (VIIA) and (VIIB), respectively:

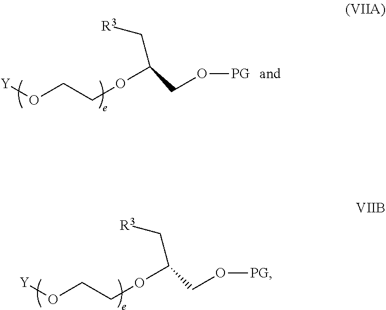

wherein PG, Y, R³, and e are as defined above.

In some embodiments, e is an integer ranging from 1 to about 24. In other embodiments, e is an integer ranging from 1 to about 20. In yet other embodiments, e is an integer ranging from 1 to about 16. In further embodiments, e is an integer ranging from 1 to about 12. In even further embodiments, e is an integer ranging from 1 to about 8. In yet further embodiments, e is an integer ranging from 1 to about 4. In yet even further embodiments, e is 2.

In some embodiments, e is an integer ranging from 1 to about 24, and Y is an alkynyl group, e.g.,

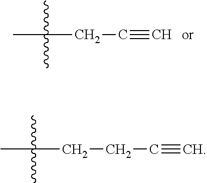

In other embodiments, e is an integer ranging from 1 to about 20, and Y is an alkynyl group, e.g.,

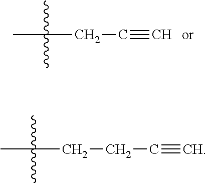

In yet other embodiments, e is an integer ranging from 1 to about 16, and Y is an alkynyl group, e.g.,

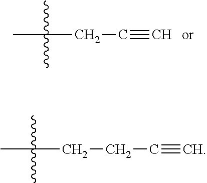

In further embodiments, e is an integer ranging from 1 to about 12, and Y is an alkynyl group, e.g.,

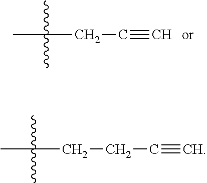

In even further embodiments, e is an integer ranging from 1 to about 8, and Y is an alkynyl group, e.g.,

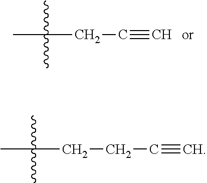

In yet further embodiments, e is an integer ranging from 1 to about 4, and Y is an alkynyl group, e.g.,

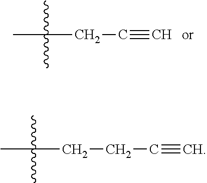

In yet further embodiments, e is an integer ranging from 1 to about 3, and Y is an alkynyl group, e.g.,

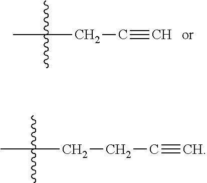

In yet even further embodiments, e is 2, and Y is an alkynyl group, e.g.,

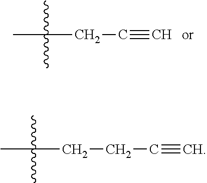

In some embodiments, e is an integer ranging from 1 to about 24, and Y is an alkyl group. In other embodiments, e is an integer ranging from 1 to about 20, and Y is an alkyl group (e.g., —CH₃). In yet other embodiments, e is an integer ranging from 1 to about 16, and Y is an alkyl group. In further embodiments, e is an integer ranging from 1 to about 12, and Y is an alkyl group. In even further embodiments, e is an integer ranging from 1 to about 8 and is an alkyl group. In yet further embodiments, e is an integer ranging from 1 to about 4, and Y is an alkyl group. In yet even further embodiments, e is 2, and Y is an alkyl group.

In some embodiments, e is an integer ranging from 1 to about 24, and Y is a methyl group. In other embodiments, e is an integer ranging from 1 to about 20, and Y is a methyl group. In yet other embodiments, e is an integer ranging from 1 to about 16, and Y is a methyl group. In further embodiments, e is an integer ranging from 1 to about 12, and Y is a methyl group. In even further embodiments, e is an integer ranging from 1 to about 8 and is a methyl group. In yet further embodiments, e is an integer ranging from 1 to about 4, and Y is a methyl group. In yet even further embodiments, e is 2, and Y is a methyl group.

In some embodiments, e is an integer ranging from 1 to about 24, and Y is an alkenyl group. In other embodiments, e is an integer ranging from 1 to about 20, and Y is an alkenyl group. In yet other embodiments, e is an integer ranging from 1 to about 16, and Y is an alkenyl group. In further embodiments, e is an integer ranging from 1 to about 12, and Y is an alkenyl group. In even further embodiments, e is an integer ranging from 1 to about 8 and is an alkenyl group. In yet further embodiments, e is an integer ranging from 1 to about 4, and Y is an alkenyl group. In yet even further embodiments, e is 2, and Y is an alkenyl group.

In some embodiments, e is an integer ranging from 1 to about 24, and Y is an acyl group. In other embodiments, e is an integer ranging from 1 to about 20, and Y is an acyl group. In yet other embodiments, e is an integer ranging from 1 to about 16, and Y is an acyl group. In further embodiments, e is an integer ranging from 1 to about 12, and Y is an acyl group. In even further embodiments, e is an integer ranging from 1 to about 8 and is an acyl group. In yet further embodiments, e is an integer ranging from 1 to about 4, and Y is an acyl group. In yet even further embodiments, e is 2, and Y is an acyl group.

In some embodiments, the intermediates of Formulas (II) through (VII) may be utilized to in the synthesis of phosphoramidite-containing monomers having the structure of any one of Formulas (VIIIA) to (VIID):

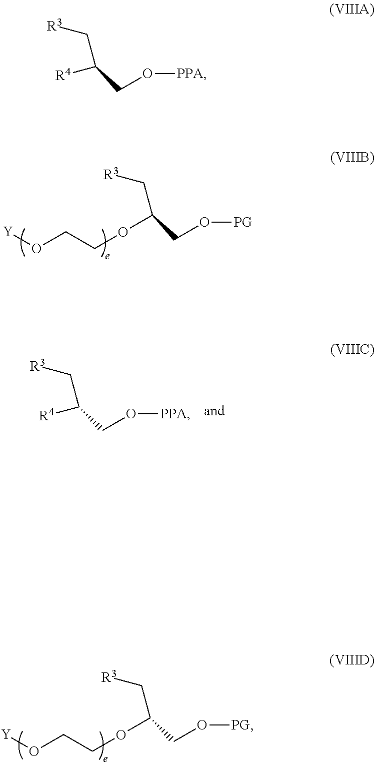

wherein Y, R³, R⁴, and e are as defined above; and where PPA is a phosphoramidite species.

In some embodiments, the phosphoramidite species has the structure:

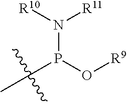

where R⁹ is a substituted or unsubstituted C₁-C₆ alkyl group terminating in a cyano moiety; and where R¹⁰ and R¹¹ are independently a branched or unbranched C₁-C₆ alkyl group.

In some embodiments, the intermediates in the synthesis of phosphoramidite-containing monomers of Formulas (IA) and (IB) have Formulas (IXA) and (IXB), respectively:

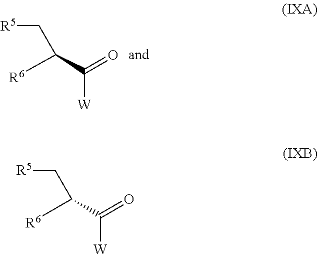

where
W is H, —O—CH₃, or —O-T, where T is an C₁-C₆ branched or unbranched alkyl group;
R⁵ is —OH, —O-trityl or a derivative or analog thereof, —O-9-phenylthioxanthyl (pixyl) or a derivative or analog thereof, —O-2-(2-nitrophenyl)prop-1-oxycarbonyl (“NPPOC”), —O-(2-nitrobenzyl) or a derivative or analog thereof, —O-(1-(2-nitrophenyl)ethyl) or a derivative or analog thereof, tetrahydropyranyl ether, ethoxyethyl ether, methallyl ether, prenyl ether, or methoxymethyl ether;
R⁶ is —OH, —O—R^{w}—Z, where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, —CH₂-Het, where “Het” is a substituted or unsubstituted 5-membered heterocyclic moiety;
provided that R⁵ and R⁶ are both not —OH.

In some embodiments, W is H.

In some embodiments, W is —O—CH₃.

In some embodiments, the “click functional group” is selected from DBCO, TCO, maleimide, —N3, tetrazine, thiol, 1,3-nitrone, hydrazine, and hydroxylamine.

In some embodiments, R⁵ is or 9-phenylthioxanthyl or a derivative or analog thereof.

In some embodiments, R⁵ is —O-trityl or a derivative or analog thereof.

In some embodiments, R⁵ has the structure:

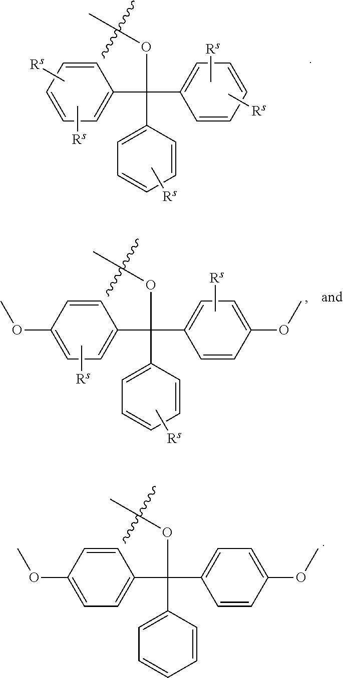

where each R^{s} is independently H, —O—C₁-C₄ alkyl or —C₁-C₄ alkyl. In some embodiments, each R^{s} is independently selected from H, —O—C₁-C₃ alkyl or —C₁-C₃ alkyl. In some embodiments, R each^{s} is independently selected from H, —O—C₁-C₂ alkyl or —C₁-C₂ alkyl. In some embodiments, each R^{s} is independently selected from H, —O—CH₃ or —CH₃.

In some embodiments, R^{w} is a substituted or unsubstituted, branched or unbranched, saturated or unsaturated alkyl moiety having between 2 and 80 carbon atoms, and which optionally includes one or more oxygen heteroatoms. In some embodiments, R^{w} is a substituted or unsubstituted, branched or unbranched, saturated or unsaturated alkyl moiety having between 2 and 60 carbon atoms, and which optionally includes one or more oxygen heteroatoms. In some embodiments, R^{w} is a substituted or unsubstituted, branched or unbranched, saturated or unsaturated alkyl moiety having between 4 and 48 carbon atoms, and which optionally includes one or more oxygen heteroatoms. In some embodiments, R^{w} is a substituted or unsubstituted, branched or unbranched, saturated or unsaturated alkyl moiety having between 4 and 24 carbon atoms, and which optionally includes one or more oxygen heteroatoms. In some embodiments, R^{w} is a substituted or unsubstituted, branched or unbranched, saturated or unsaturated alkyl moiety having between 4 and 12 carbon atoms, and which optionally includes one or more oxygen heteroatoms.

In some embodiments, R^{w} is an unsubstituted alkyl moiety having between 2 and 80 carbon atoms, and which optionally includes one or more oxygen heteroatoms. In some embodiments, R^{w} is an unsubstituted alkyl moiety having between 2 and 60 carbon atoms, and which optionally includes one or more oxygen heteroatoms. In some embodiments, R^{w} is an unsubstituted alkyl moiety having between 4 and 48 carbon atoms, and which optionally includes one or more oxygen heteroatoms. In some embodiments, R^{w} is an unsubstituted alkyl moiety having between 4 and 24 carbon atoms, and which optionally includes one or more oxygen heteroatoms. In some embodiments, R^{w} is an unsubstituted alkyl moiety having between 4 and 12 carbon atoms, and which optionally includes one or more oxygen heteroatoms.

In some embodiments, R^{w} includes at least one PEG group, at least two PEG groups, at least three PEG groups, at least four PEG groups, at least 8 PEG groups, at least 12 PEG groups, or at least 24 PEG groups.

In some embodiments, R^{w} includes at least one PEG group, at least two PEG groups, at least three PEG groups, at least four PEG groups, at least 8 PEG groups, at least 12 PEG groups, or at least 24 PEG groups; and where Z is an alkyl group, e.g., a C₁-C₄ alkyl group, methyl, or ethyl.

In some embodiments, R^{w} includes at least one PEG group, at least two PEG groups, at least three PEG groups, at least four PEG groups, at least 8 PEG groups, at least 12 PEG groups, or at least 24 PEG groups; and where Z is an alkenyl group, e.g., —CH═CH₂, or —CH₂—CH═CH₂.

In some embodiments, R^{w} includes at least one PEG group, at least two PEG groups, at least three PEG groups, at least four PEG groups, at least 8 PEG groups, at least 12 PEG groups, or at least 24 PEG groups; and where Z is an alkynyl group, e.g.,

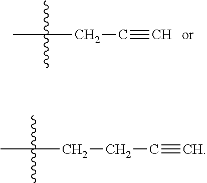

In some embodiments, R^{w} includes at least one PEG group, at least two PEG groups, at least three PEG groups, at least four PEG groups, at least 8 PEG groups, at least 12 PEG groups, or at least 24 PEG groups; and where Z is an acyl group.

In some embodiments, R^{w} includes at least one PEG group, at least two PEG groups, at least three PEG groups, at least four PEG groups, at least 8 PEG groups, at least 12 PEG groups, or at least 24 PEG groups; and where Z includes a substituted heterocyclic moiety. In some embodiments, the heterocyclic moiety is substituted with an alkylaryl group. In some embodiments, the substituted heterocyclic moiety includes a triazole. In some embodiments, the triazole is substituted with an alkylaryl group. In some embodiments, the triazole is substituted with —CH₂—CH₂—O-Ph. In some embodiments, the triazole is substituted with —CH₂—CH₂—O-Bz.

In some embodiments, R⁵ is —O-trityl or a derivative or analog thereof; and R⁶ is —O—R^{w}—Z, where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, or —CH₂-alkynyl. In some embodiments, R⁵ is —O-trityl or a derivative or analog thereof; and R⁶ is —O—R^{w}—Z, where R^{w} is a substituted or unsubstituted, branched or unbranched, saturated or unsaturated alkyl group having between 1 and 48 carbon atoms, and which optionally includes one or more oxygen heteroatoms, and where Z is alkyl, alkenyl, alkynyl, or —CH₂-alkynyl. In some embodiments, R⁵ is —O-trityl or a derivative or analog thereof; and R⁶ is —O—R^{w}—Z, where R^{w} is a substituted or unsubstituted, branched or unbranched, saturated or unsaturated alkyl group having between 1 and 24 carbon atoms, and which optionally includes one or more oxygen heteroatoms, and Z is alkyl, alkenyl, alkynyl, or —CH₂-alkynyl.

In some embodiments, R⁵ is —O-trityl or a derivative or analog thereof; and R⁶ is —O—R^{w}—Z, where R^{w} is an unsubstituted alkyl group having between 1 and 60 carbon atoms, and which optionally includes one or more oxygen heteroatoms, and where Z is alkyl, alkenyl, alkynyl, or —CH₂-alkynyl. In some embodiments, R⁵ is —O-trityl or a derivative or analog thereof; and R⁶ is —O—R^{w}—Z, where R^{w} is an unsubstituted alkyl group having between 1 and 48 carbon atoms, and which optionally includes one or more oxygen heteroatoms, and where Z is alkyl, alkenyl, alkynyl, or —CH₂-alkynyl. In some embodiments, R⁵ is —O-trityl or a derivative or analog thereof; and R⁶ is —O—R^{w}—Z, where R^{w} is an unsubstituted alkyl group having between 1 and 24 carbon atoms, and which optionally includes one or more oxygen heteroatoms, and where Z is alkyl, alkenyl, alkynyl, or —CH₂-alkynyl.

In some embodiments, R⁵ is —O-trityl or a derivative or analog thereof; and R^{w} includes at least one PEG group, at least two PEG groups, at least three PEG groups, at least four PEG groups, at least 8 PEG groups, at least 12 PEG groups, or at least 24 PEG groups.

In some embodiments, R⁵ is —O-trityl or a derivative or analog thereof; R^{w} includes at least one PEG group, at least two PEG groups, at least three PEG groups, at least four PEG groups, at least 8 PEG groups, at least 12 PEG groups, or at least 24 PEG groups; and where Z is an alkyl group, e.g., a C₁-C₄ alkyl group, methyl, or ethyl.

In some embodiments, R⁵ is —O-trityl or a derivative or analog thereof; R^{w} includes at least one PEG group, at least two PEG groups, at least three PEG groups, at least four PEG groups, at least 8 PEG groups, at least 12 PEG groups, or at least 24 PEG groups; and where Z is an alkenyl group, e.g., —CH═CH₂, or —CH₂—CH═CH₂.

In some embodiments, R⁵ is —O-trityl or a derivative or analog thereof; R^{w} includes at least one PEG group, at least two PEG groups, at least three PEG groups, at least four PEG groups, at least 8 PEG groups, at least 12 PEG groups, or at least 24 PEG groups; and where Z is an alkynyl group, e.g.,

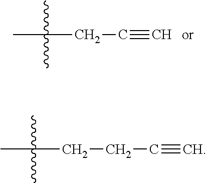

In some embodiments, R⁵ is —O-trityl or a derivative or analog thereof; R^{w} includes at least one PEG group, at least two PEG groups, at least three PEG groups, at least four PEG groups, at least 8 PEG groups, at least 12 PEG groups, or at least 24 PEG groups; and where Z is an acyl group.

In some embodiments, R⁵ is —O-trityl or a derivative or analog thereof; R^{w} includes at least one PEG group, at least two PEG groups, at least three PEG groups, at least four PEG groups, at least 8 PEG groups, at least 12 PEG groups, or at least 24 PEG groups; and where Z includes a substituted heterocyclic moiety. In some embodiments, the heterocyclic moiety is substituted with an alkylaryl group. In some embodiments, the substituted heterocyclic moiety includes a triazole, e.g., a 1,2,4-triazole. In some embodiments, the triazole is substituted with an alkylaryl group. In some embodiments, the triazole is substituted with —CH₂—CH₂—O-Ph. In some embodiments, the triazole is substituted with —CH₂—CH₂—O-Bz.

Non-limiting examples of compounds having any one of Formulas (IXA) and (IXB) include:

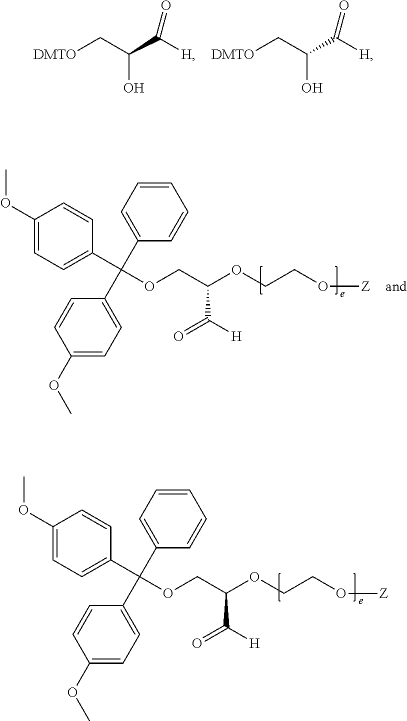

where DMTO is 4,4′-dimethoxytrityl ether; Z is as defined above; and e is an integer ranging from 1 to 24. In some embodiments, Z is allyl or alkyl. In some embodiments, e is 2. In other embodiments, is 3. In other embodiments, e is 4. In yet other embodiments, e is 8. In some embodiments, e is 4 and Z is allyl or alkyl. Also contemplated are compounds which include an ether of 9-phenylthioxanthyl or a derivative or analog thereof in place of DMTO.

In some embodiments, the intermediates of Formulas (IXA) and (IXB) have the structure of any one of Formulas (XA) and (XB):

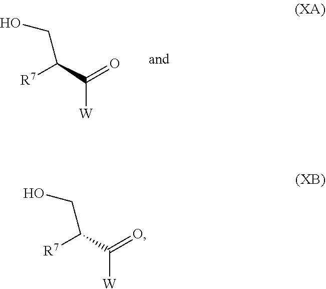

where W is as defined above; and
R⁷ is —O—R^{w}—Z, where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, acyl, a “click functional group,” or a substituted or unsubstituted 5-membered heterocyclic moiety.

In some embodiments, R⁷ has any one of Formulas (XIIIA) and (XIIIB):

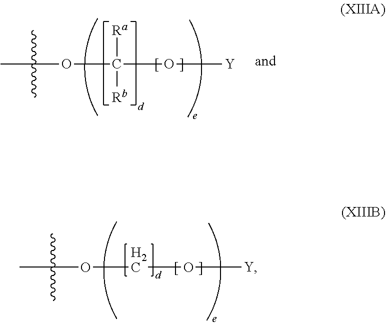

where
R^{a} and R^{b} are independently H or a C₁-C₄ alkyl;
Y is alkyl, alkenyl, alkynyl, acyl, —CH₂-alkynyl, a “click functional group,” -Het, or —CH₂-Het, where “Het” is a substituted or unsubstituted 5-membered heterocyclic moiety;
d is an integer ranging from between 1 to about 10; and
e is an integer ranging from between 1 to about 36.

In some embodiments, d is an integer ranging from 1 to about 8. In other embodiments, d is an integer ranging from 1 to about 6. In yet other embodiments, d is an integer ranging from 1 to about 2. In further embodiments, d is 2. In yet even further embodiments, d is 1.

In some embodiments, e is an integer ranging from 1 to about 24. In other embodiments, e is an integer ranging from 1 to about 20. In yet other embodiments, e is an integer ranging from 1 to about 16. In further embodiments, e is an integer ranging from 1 to about 12. In even further embodiments, e is an integer ranging from 1 to about 8. In yet further embodiments, e is an integer ranging from 1 to about 4. In some embodiments, e is 4. In some embodiments, e is 3. In some embodiments, e is 2. In some embodiments, e is 1.

In some embodiments, the heterocyclic moiety is substituted with an alkylaryl group. some embodiments, the substituted heterocyclic moiety includes a triazole. In some embodiments, the triazole is substituted with an alkylaryl group. In some embodiments, the triazole is substituted with —CH₂—CH₂—O-Ph. In some embodiments, the triazole is substituted with —CH₂—CH₂—O-Bz.

In some embodiments, the intermediates of Formulas (IXA) and (IXB) have the structure of any one of Formulas (XIA) and (XIB):

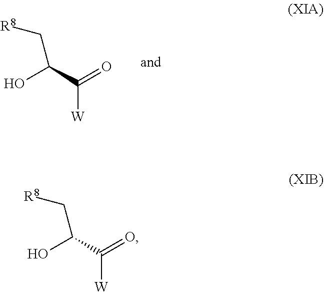

where W is as defined above; and

R⁸ is —OH, —O-trityl or a derivative or analog thereof, —O-9-phenylthioxanthyl (pixyl) or a derivative or analog thereof, —O-2-(2-nitrophenyl)prop-1-oxycarbonyl (“NPPOC”), —O-(2-nitrobenzyl) or a derivative or analog thereof, —O-(1-(2-nitrophenyl)ethyl) or a derivative or analog thereof, —O-1-(2-fluorophenyl)-4-methoxypiperidin-4-yl, —O-[(chloro-4-methyl)phenyl]-4′-methoxypiperidin-4-yl, tetrahydropyranyl ether, ethoxyethyl ether, methallyl ether, prenyl ether, or methoxymethyl ether.

In some embodiments, R⁸ is —O-pixyl or a derivative or analog thereof. Non-limiting examples of derivatives or analogs of pixyl are described in United States Patent Application 2007/0276139 the disclosure of which is hereby incorporated by reference herein in its entirety.

In some embodiments, R⁸ is —O-trityl or a derivative or analog thereof.

In some embodiments, R⁸ has the structure:

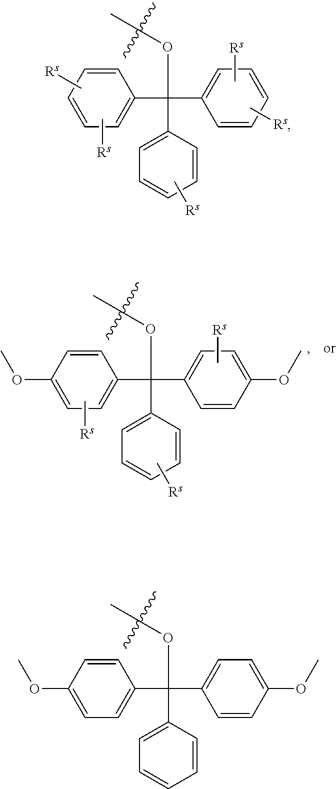

where each R^{s} is independently H, —O—C₁-C₄ alkyl or —C₁-C₄ alkyl. In some embodiments, each R^{s} is independently selected from H, —O—C₁-C₃ alkyl or —C₁-C₃ alkyl. In some embodiments, each R^{s} is independently selected from H, —O—C₁-C₂ alkyl or —C₁-C₂ alkyl. In some embodiments, each R^{s} is independently selected from H, —O—CH₃ or —CH₃.

In some embodiments, each R⁸ is 4,4′-dimethoxytrityl ether or 4-methoxytrityl ether.

In some embodiments, the intermediates of Formulas (IXA) or (IXB) may be utilized in the synthesis of phosphoramidite-containing monomers having the structure of any one of Formulas (XIIA) to (XIID):

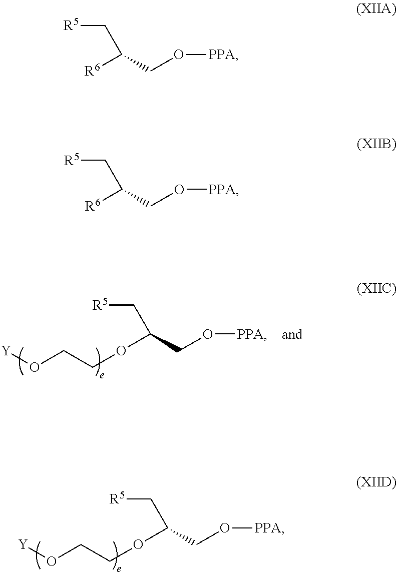

wherein Y, R⁵, R6⁴, and e are as defined above; and where PPA is a phosphoramidite species.

In some embodiments, phosphoramidite species has the structure:

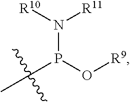

where R⁹ is a substituted or unsubstituted C₁-C₆ alkyl group terminating in a cyano moiety; and where R¹⁰ and R¹¹ are independently a branched or unbranched C₁-C₆ alkyl group.

In some embodiments, the present disclosure provides for compounds having any one of Formulas (XIIIA) or (XIIIB);

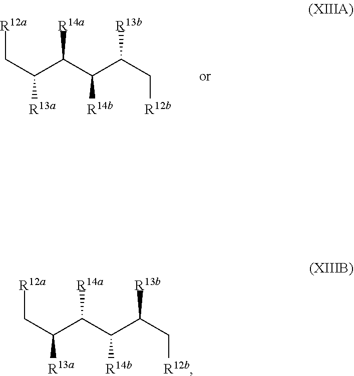

wherein
R^{12a} and R^{12b} are independently —OH, —O-trityl or a derivative or analog thereof, —O-9-phenylthioxanthyl (pixyl) or a derivative or analog thereof, —O-2-(2-nitrophenyl)prop-1-oxycarbonyl (“NPPOC”), —O-(2-nitrobenzyl) or a derivative or analog thereof, —O-(1-(2-nitrophenyl)ethyl) or a derivative or analog thereof, tetrahydropyranyl ether, ethoxyethyl ether, methallyl ether, prenyl ether, or methoxymethyl ether;
R^{13a} and R^{13b} are independently —O—R^{w}—Z, where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, or —CH₂-Het, where “Het” is a substituted or unsubstituted 5-membered heterocyclic moiety;
R^{14a} and R^{14b} are —OH;
or where R^{12a} and R^{14a} and/or R^{12b} and R^{14b} taken together may be

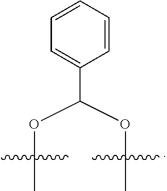 [SMILES: *OC(O*)c1ccccc1]

In some embodiments, the compounds of Formulas (XIIIA) and (XIIIB) serve as starting materials for the preparation of regioisomerically pure phosphoramidite-containing monomers. As described herein, such monomers may be polymerized into polymers or copolymers; and where such formed polymers or copolymers may be incorporated into XNTP molecules.

In some embodiments, R^{w} includes between about 4 and about 12 carbon atoms. In other embodiments, R^{w} includes between about 6 and about 12 carbon atoms. In yet other embodiments, R^{w} includes between about 9 and about 12 carbon atoms.

In some embodiments, R^{w} includes between about 4 and about 12 carbon atoms, and further includes at least 2 heteroatoms, e.g., at least 2 oxygen heteroatoms, at least 3 oxygen heteroatoms, at least 4 oxygen heteroatoms, etc. In other embodiments, R^{w} includes between about 6 and about 12 carbon atoms, and further includes at least 2 heteroatoms, e.g., at least 2 oxygen heteroatoms, at least 3 oxygen heteroatoms, at least 4 oxygen heteroatoms, etc. In yet other embodiments, R^{w} includes between about 8 and about 12 carbon atoms, and further includes at least 2 heteroatoms, e.g., at least 2 oxygen heteroatoms, at least 3 oxygen heteroatoms, at least 4 oxygen heteroatoms, etc.

In some embodiments, R^{12a} and R^{14a} taken together is

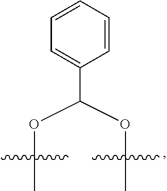 [SMILES: *OC(O*)c1ccccc1]

R^{12b} and R^{14b} taken together is

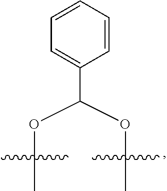 [SMILES: *OC(O*)c1ccccc1]

and where Z is alkyl, alkenyl, alkynyl, or —CH₂-alkynyl.

In some embodiments, Z is alkyl, alkenyl, alkynyl, —CH₂-alkynyl; and R^{14a} and R^{14b} are each —OH. In some embodiments, Z is alkyl, alkenyl, alkynyl, —CH₂-alkynyl; R^{14a} and R^{14b} are each —OH; and R^{12a} and R^{12b} are each —O-trityl or a derivative or analog thereof. In some embodiments, Z is alkyl, alkenyl, alkynyl, —CH₂-alkynyl; R^{14a} and R^{14b} are each —OH; and R^{12a} and R^{12b} are each —O— pixyl or a derivative or analog thereof.

Non-limiting examples of compounds having any one of Formulas (XIIIA) or (XIIIB) include:

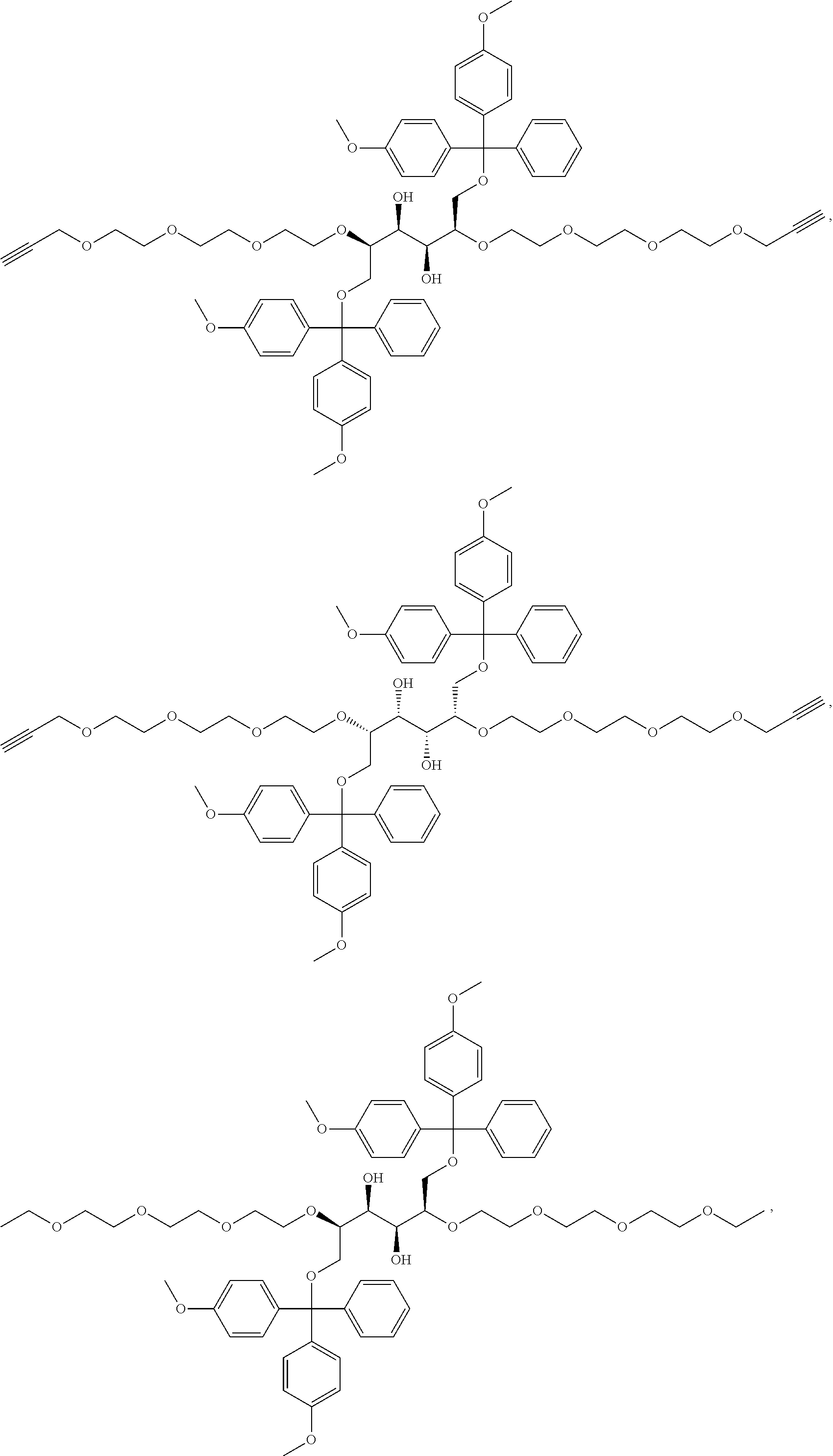

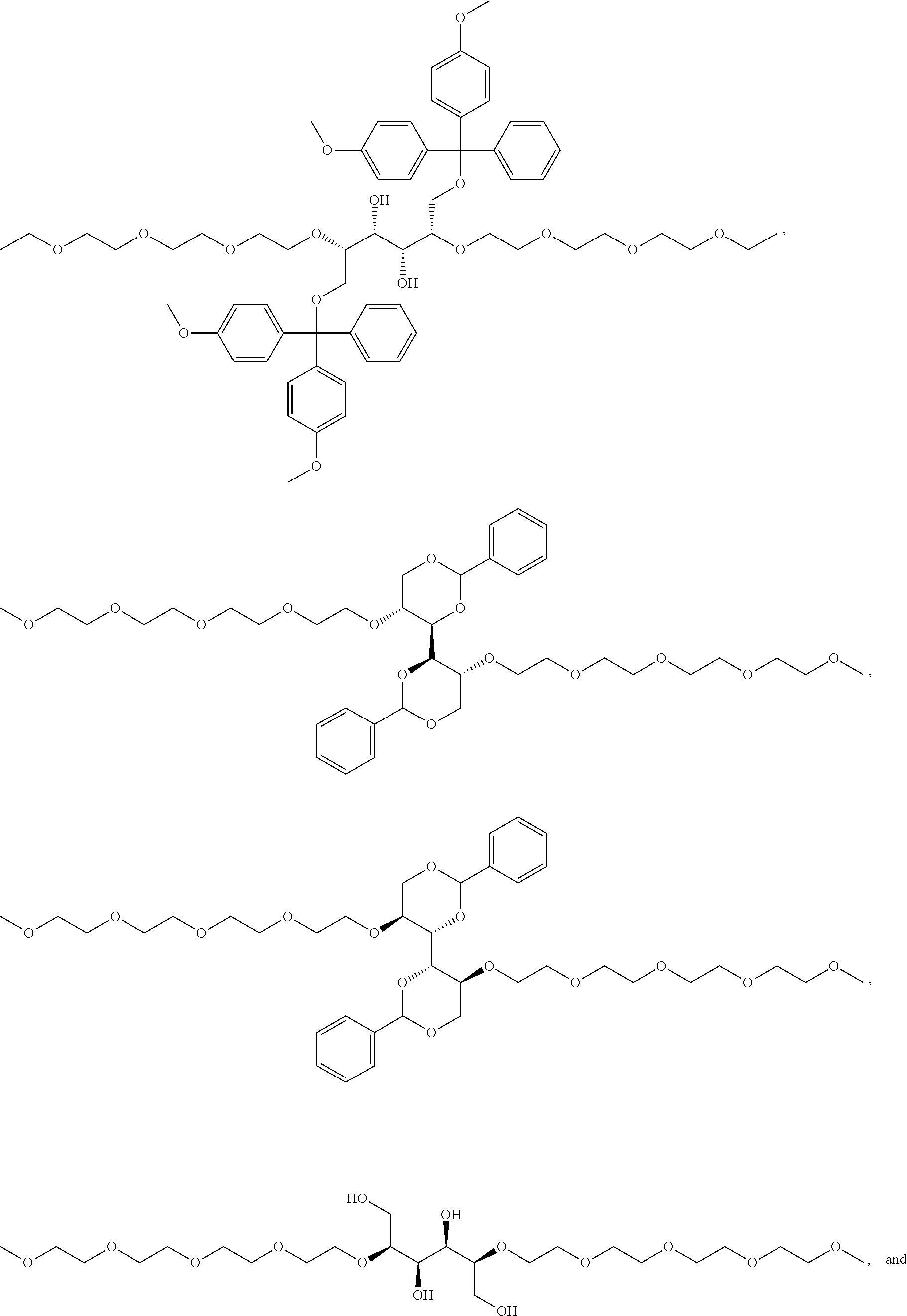

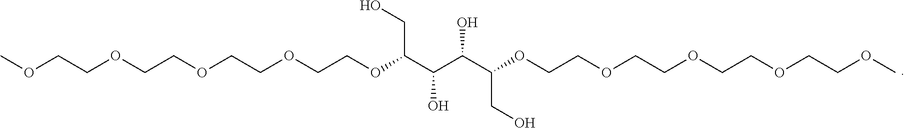

### Synthesis of Intermediates and Phosphoramidite-Containing Monomers

The present disclosure also provides methods of synthesizing phosphoramidite-containing monomers. In some embodiments, the synthetic methods result in high yields of regioisomerically and enantiomerically pure phosphoramidite-containing monomers. The present disclosure also provides methods of synthesizing intermediates of phosphoramidite-containing monomers, including any of the intermediates described herein.

Scheme 1 depicts a method of synthesizing phosphoramidite-containing monomers (see also Example 1, herein). Classical silyl groups such as TIPS or TBDPS have been used to transiently protect hydroxyl functions of glycerol in the course of the synthesis of chiral phosphoramidite-containing monomers (see Scheme 1, compound 1). Migration of the introduced silyl group has been observed between two neighboring hydroxyl functions resulting in a conversion of compound 3 to 4 or vice versa. As a result, mixtures of regioisomers are obtained, which are difficult to separate by chromatography. The resulting phosphoramidite-containing monomers thus include the respective regioisomeric impurities. When starting from TIPS-protected compound 3 the corresponding phosphoramidite-containing monomer 1 was obtained with an impurity of up to 15% of phosphoramidite-containing monomer 2. When starting from TBDPS-protected 3 the corresponding phosphoramidite-containing monomer 1 was obtained with up to 6% of phosphoramidite-containing monomer 2.

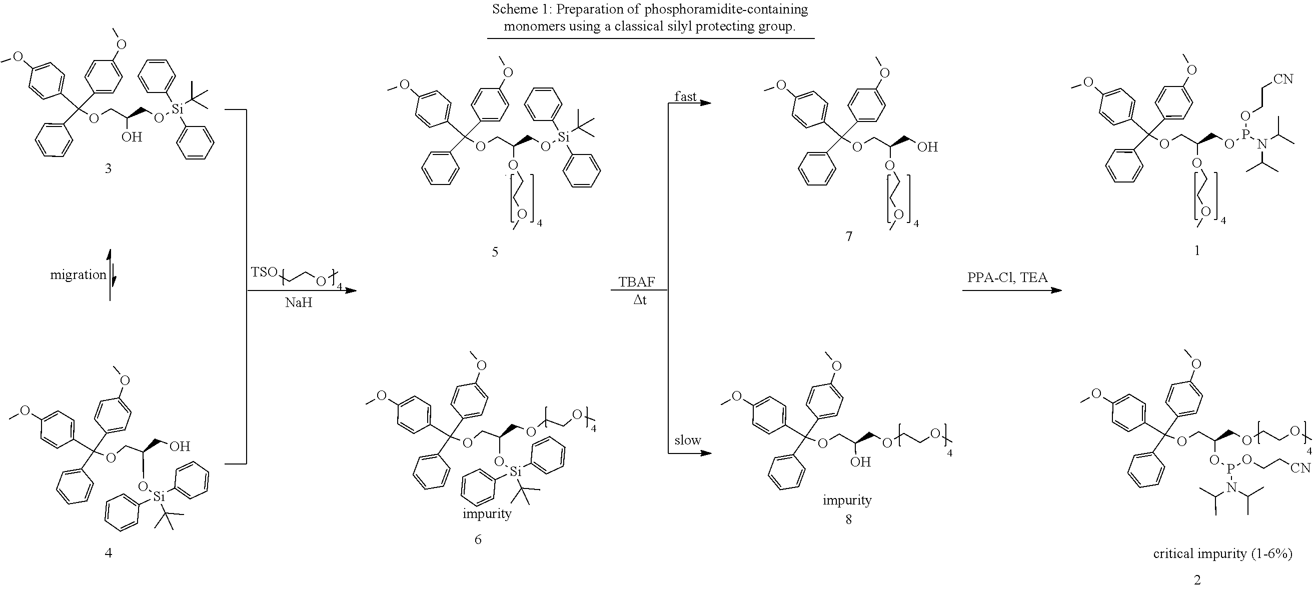

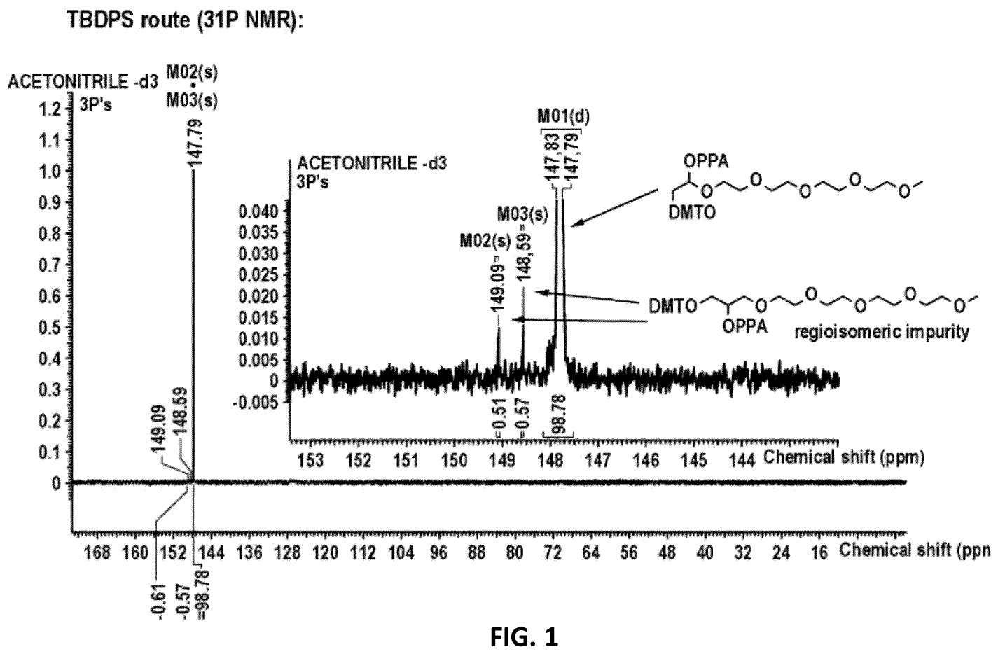

The amount of the regioisomeric impurity phosphoramidite-containing monomer may, in some embodiments, be reduced by exploiting a kinetic resolution in the deprotection step of TBDPS-protected compounds 5 and 6 (mixture). Employing TBAF, pegylated compound 5 deprotects faster than compound 6 (see Scheme 2). For this, the reaction needs to be stopped before full conversion is reached. Table 1 shows the dependencies of deprotection time, yield and regioisomeric impurity in the final phosphoramidite-containing monomer product. With decreasing reaction time, the impurity could be lowered to 0.7% (determined by 31P-NMR); however, the yield of intermediate 7 and 8 decreased from 86% to 41%. This effect could be explained by the steric hindrance and potential n-stacking of the neighboring DMT and TBDPS groups in pegylated compound 6. The bulky TBAF reagent might encounter stronger steric repulsion with compound 6 than with compound 5 (see FIG. 1).

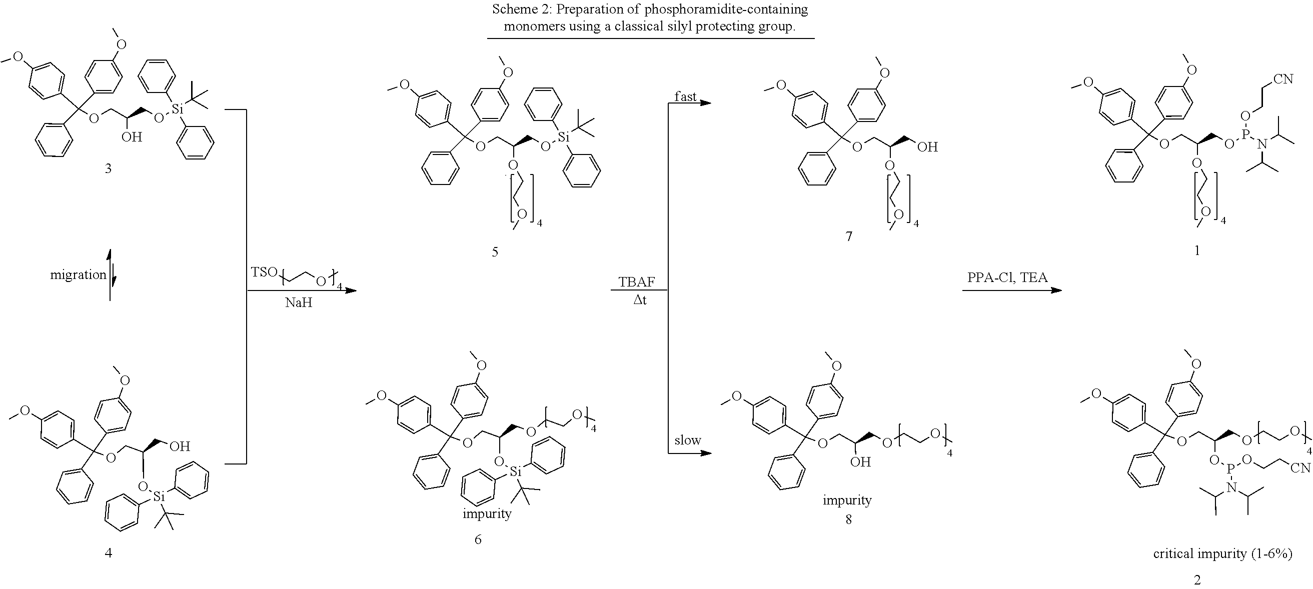

TABLE 1

| | Silyl deprotection | | Rel. amount of | |
|---|---|---|---|---|
| | time | | regioisomer 2 in | Yield |
| | (5 + 6 converted | | final product 1 | (isolated |
| Experiment | to 7 + 8) | | (31P NMR area-%) | 7 + 8) |
| 1 | over night | | 4.5% | 86% |
| 2 | 3 | h | 1.6% | 83% |
| 3 | 1 | h | 0.9% | 79% |
| 4 | 20 | min | 0.7% | 41% |

Applicants have surprisingly discovered that the selection of certain protecting groups facilitate the synthesis of a phosphoramidite-containing monomer that regiochemically and enantiomerically pure. Synthetic Schemes 3 and 4 illustrate methods of preparing such stereochemically pure phosphoramidite-containing monomers and intermediates thereof, such as intermediates having any one of Formulas (IIA) or (IIB). The person of ordinary skill in the art will appreciate that Schemes 3 and 4 utilize starting materials (compounds A and A′) having different stereochemical centers (R vs. S). Notably, the stereochemical centers in each of Schemes 3 and 4 are retained during each step of the synthesis to yield a phosphoramidite-containing monomer (compounds F and F′) having the same stereochemistry as the starting material (compounds A and A′, respectively).

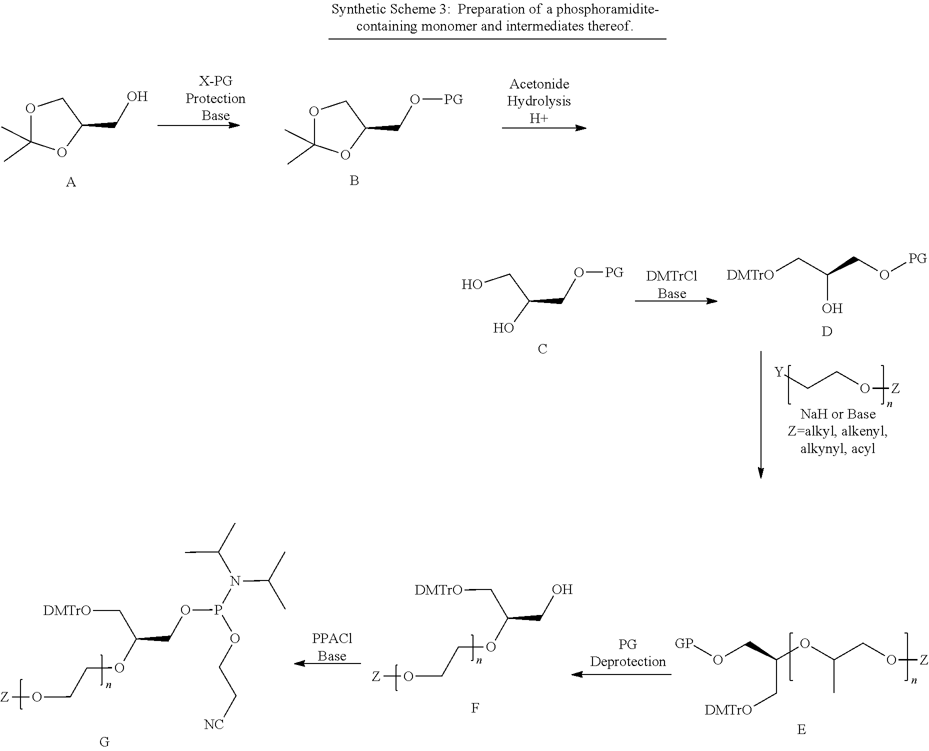

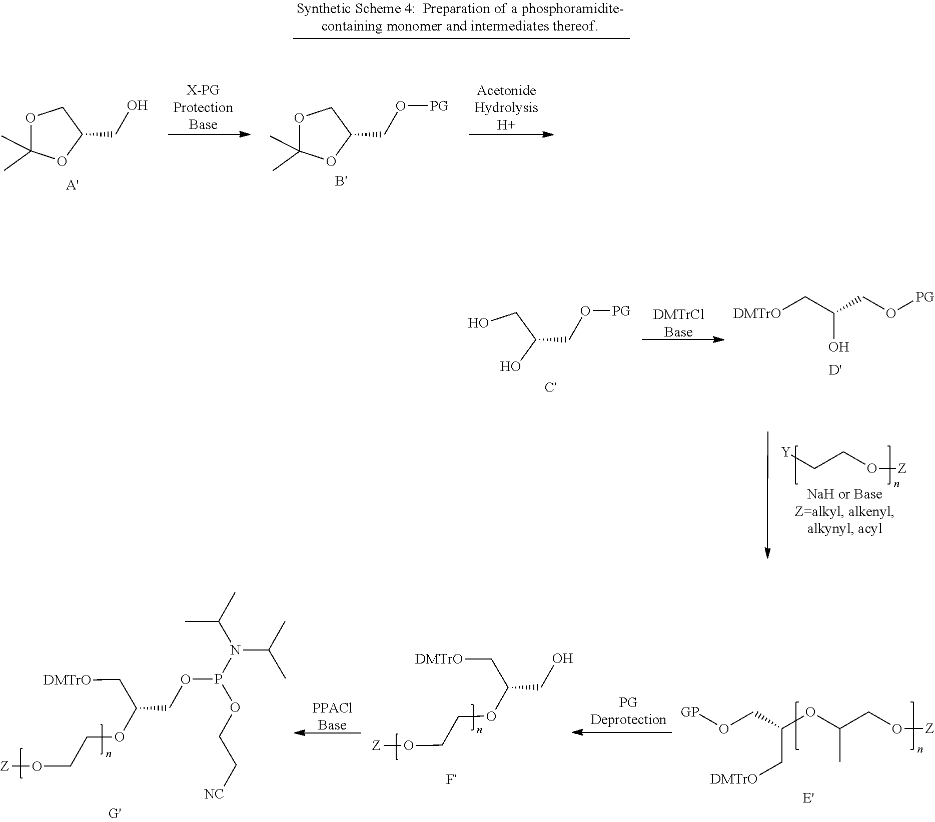

In general, compounds A and A′ may be reacted with a protecting group, e.g., Br—CH₂—CH═CH₂, in the presence of base (e.g., a non-nucleophilic base, such as NaH) to form protected compounds B and B′, respectively. Examples of suitable protecting groups are described in Wuts, P. G. M., & Greene, T. W. (2007), the disclosure of which is hereby incorporated by reference herein in its entirety. Other suitable protecting groups are disclosed within the Examples provided herein. Other non-nucleophilic bases include N,N-Diisopropylethylamine (DIPEA); silicon-based amides, such as sodium and potassium bis(trimethylsilyl)amide (NaHMDS and KHMDS, respectively); 1,8-Diazabicycloundec-7-ene (DBU); and 2,6-Di-tert-butylpyridine, and lithium tetramethylpiperidide (LiTMP or harpoon base).

Compounds B and B′ may then be reacted with an acid (e.g., a weak acid, such as acetic acid) to form compounds C and C′. The skilled artisan will appreciate that some protecting groups are more sensitive to acids than others. Other suitable acids include, but are not limited to, formic acid, benzoic acid, oxalic acid, hydrofluoric acid, nitrous acid, hydrochloric acid, sulfurous acid, and phosphoric acid. The choice of acid depends on the protecting group introduced. Subsequently, compounds C and C′ may be reacted with trityl-chloride or a derivative or analog thereof in the presence of a base (e.g., TEA) to provide the tritylated compounds D and D′. A side chain (e.g., —O—R^{w}—Z, as described herein) may then by introduced to tritylated compounds D and D′ in the presence of NaH or another base (e.g., a strong base, such as NaOH or KOtBu) to provide compounds E and E′.

Compounds E and E′ may then be deprotected according to methods known to those of ordinary skill in the art to provide orthogonally deprotected compounds F and F. For example, in some embodiments a SEM protecting group or a 2-O-Triisopropylsilyloxymethyl can be orthogonally deprotected using tetrabutylammonium fluoride. In other embodiments, a 2′-thiomorpholine-4-carbothioate protecting group can be removed by ethylenediamine. In yet other embodiments, a benzyl group can be oxidized by 2,3-Dichloro-5,6-dicyano-1,4-benzoquinone or reduced in the presence of Pd/C or Pd(OH)₂/C. In yet other embodiments, an azidomethyl protecting group may be removed by a mild Staudinger reduction. In further embodiments, an allyl group may be cleaved by Pd(0) and morpholine.

Finally, a phosphoramidite species (including any of those recited herein) may be coupled to compounds F and F in the presence of base (e.g., TEA) to provide the phosphoramidite-containing monomers.

In some embodiments, when group Z in Schemes 3 and 4 is alkynyl, the group Z may react with another substrate in a “click chemistry” reaction. For instance, when group Z in Schemes 3 and 4 is alkynyl, the group Z may react with a substrate having an azide group, such as azidoethylbenzoate. Yet other suitable substrates having an azide group include, but are not limited to, azido pentaerythritol, azido benzoic acid/esters, diether phosphoryl azides, crown ether azide, azide sugars, and azide aminoacids.

In some embodiments, the present disclosure provides a method of synthesizing a regioisomerically and/or enantiomerically pure monomer having any one of Formulas (VIIIA) to (VIIID), the method comprising:
(a) preparing a compound having any one of Formulas (IIIA) and (IIIB) from 2,3-isopropylidene-sn-glycerol, wherein the compound having any one of Formulas (IIIA) and (IIB) has the structure:

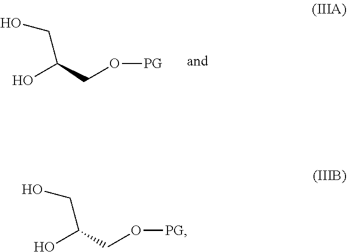

wherein
PG is

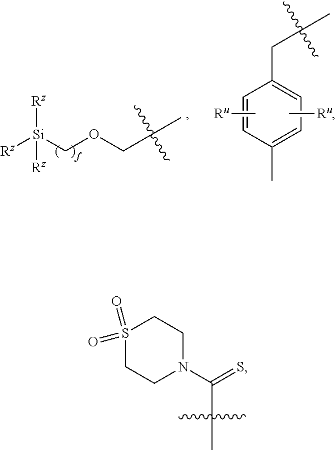

—CH₂—S—CH₃, —CH₂—N₃, or —CH₂—CH₂═CH₂;
f is 0 or 2;
each R^{u} is independently H, —O—C₁-C₄ alkyl or —C₁-C₄ alkyl; and
each R^{z} is independently a branched or unbranched C₁-C₆ alkyl group;
(b) reacting trityl chloride or a derivative or analog thereof with the compound having any one of Formulas (IIIA) and (IIIB) to provide a compound having any one of Formulas (VA) and (VB), respectively, wherein the compound having any one of Formulas (VA) and (VB) has the structure:

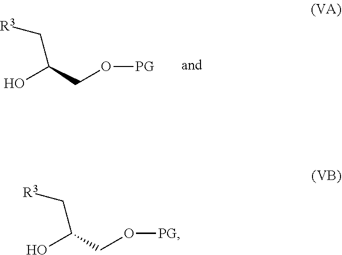

wherein PG is as defined above, and wherein R³ is —O-trityl or a derivative or analog thereof;
(c) reacting a reagent having Formula (XIV) with the compound having any one of Formulas (VA) and (VB) to provide a conjugate having any one of Formulas (VIA) and (VIB) respectively;
wherein the reagent having Formula (XIV) has the structure:

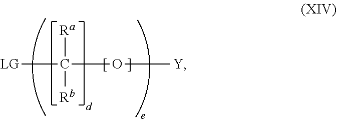

where
R^{a} and R^{b} are independently H or a C₁-C₄ alkyl;
Y is alkyl, alkenyl, alkynyl, acyl, —CH₂-alkynyl, a “click functional group,” -Het, or —CH₂-Het, where “Het” is a substituted or unsubstituted 5-membered heterocyclic moiety;
d is an integer ranging from between 1 to about 10;
e is an integer ranging from between 1 to about 36; and
LG is a leaving group;

wherein the conjugate having any one of Formulas (VIA) and (VIB) has the structure:

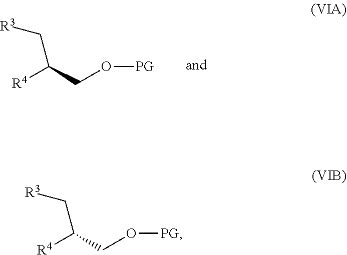

wherein PG and R³ are as defined above; and
R⁴ has Formula (XIIIA):

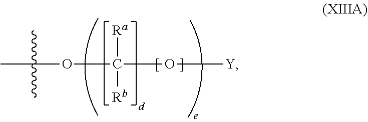

where R^{a} and R^{b}, Y, d, and e are as defined above;
(d) deprotecting the conjugate having any one of Formulas (VIA) and (VIB) to provide the respective deprotected conjugate; and
(e) coupling a compound having Formula (XV) to the deprotected conjugate to provide the regioisomerically and/or enantiomerically pure monomer having any one of Formulas (VIIIA) to (VIIID), respectively, wherein the compound having Formula (XV) has the structure:

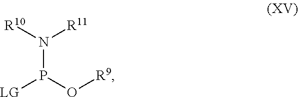

here LG is a leaving group, R⁹ is a substituted or unsubstituted C₁-C₆ alkyl group terminating in a cyano moiety; and where R¹⁰ and R¹¹ are independently a branched or unbranched C₁-C₆ alkyl group;

and where the compounds having any one of Formulas (VIIIA) to (VIIID) have the structure:

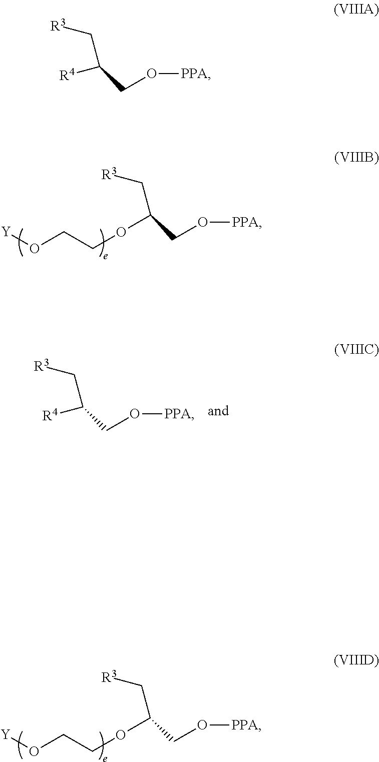

wherein Y, R³, R⁴, and e are as defined above; and where PPA is a phosphoramidite species.

In some embodiments, the present disclosure provides methods of converting compounds having any one of Formulas (XA) and (XB) to compounds having any one of Formulas (IXA) and (IXB). In these embodiments, the method comprises obtaining a compound having any one of Formulas (XA) and (XB) and reacting it with trityl chloride (or a derivative or analog thereof) a solvent (e.g., DCM) (see Example 9, herein).

In some embodiments, the present disclosure provides methods of converting compounds having any one of Formulas (XIA) and (XIB) to compounds having any one of Formulas (IXA) and (IXB). In these embodiments, the method comprises obtaining a compound having any one of Formulas (XIA) and (XIB) and reacting it with a base (e.g., NaH) in a solvent (e.g., THF) in the presence of a side chain, such as a side chain having Formula (XIV) (see Examples 10 and 11, herein).

Another aspect of the present disclosure is a method of preparing a compound having any one of the structures:

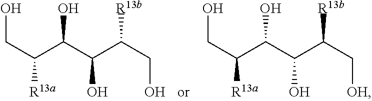

where R^{13a} and R^{13b} are independently —O—R^{w}—Z, and where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, or —CH₂-Het, where “Het” is a substituted or unsubstituted 5-membered heterocyclic moiety;
the method comprising:
introducing a lower alcohol and an acid to a starting material having any one of the structures:

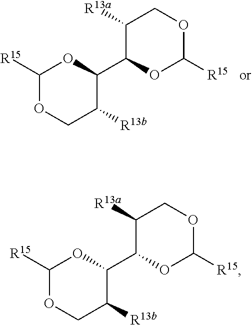

where R¹⁵ is a substituted or unsubstituted 5- or 6-membered aromatic or heteroaromatic group. Specific methods of preparing these compounds are described in Examples 16 and 17 herein.

In some embodiments, the lower alcohol is selected from the group consisting of methanol and ethanol. In some embodiments, the acid is hydrochloric acid. In some embodiments, Z is alkyl, alkenyl, alkynyl, or —CH₂-alkynyl. In some embodiments, R^{w} includes between 4 and 16 carbon atoms. In some embodiments, wherein R^{w} includes between 6 and 12 carbon atoms. In some embodiments, R^{w} includes between 8 and 12 carbon atoms. In some embodiments, R^{w} further includes at least two oxygen heteroatoms.

Another aspect of the present disclosure is a method of preparing a compound having any one of the structures:

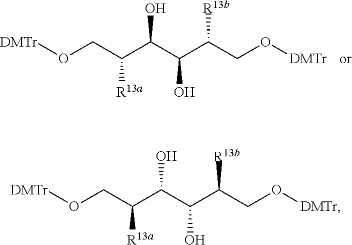

wherein R^{13a} and R^{13b} are independently —O—R^{w}—Z, and where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, or —CH₂-Het, where “Het” is a substituted or unsubstituted 5-membered heterocyclic moiety;
the method comprising:
reacting 4,4′Dimethoxytrityl chloride with a compound having any one of the following structures:

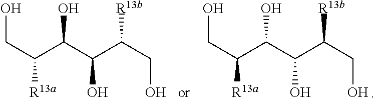

Specific methods of preparing these compounds are described in Examples 16 and 17 herein.

In some embodiments, Z is alkyl, alkenyl, alkynyl, or —CH₂— alkynyl. In some embodiments, R^{w} includes between 4 and 16 carbon atoms. In some embodiments, wherein R^{w} includes between 6 and 12 carbon atoms. In some embodiments, R^{w} includes between 8 and 12 carbon atoms. In some embodiments, R^{w} further includes at least two oxygen heteroatoms.

In another aspect of the present disclosure is a method of preparing a compound having any one of Formulas (IXA) and (IXB), respectively:

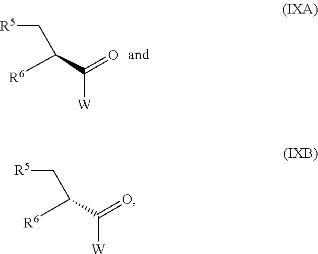

where
W is H;
R⁵ is-O-trityl;
R⁶—O—R^{w}—Z, where R^{w} is a substituted or unsubstituted, branched, or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, or —CH₂-Het, where “Het” is a substituted or unsubstituted 5-membered heterocyclic moiety;
the method comprising oxidatively cleaving a compound having any one of the structures:

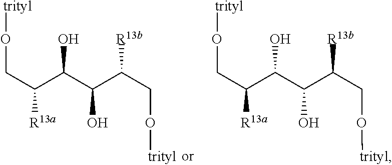

wherein R^{13a} and R^{13b} are independently —O—R^{w}—Z, and where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, or —CH₂-Het, where “Het” is a substituted or unsubstituted 5-membered heterocyclic moiety;

wherein the oxidative cleavage is performed in the presence sodium metaperiodate (NaIO₄) and a base; and wherein R⁶, R^{13a}, and R^{13b} are the same.

Specific methods of preparing these compounds are described in Examples 16 and 17 herein.

In some embodiments, Z is alkyl, alkenyl, alkynyl, or —CH₂-alkynyl. In some embodiments, R^{w} includes between 4 and 16 carbon atoms. In some embodiments, wherein R^{w} includes between 6 and 12 carbon atoms. In some embodiments, R^{w} includes between 8 and 12 carbon atoms. In some embodiments, R^{w} further includes at least two oxygen heteroatoms.

In some embodiments, the synthesized compound has one of the following structures:

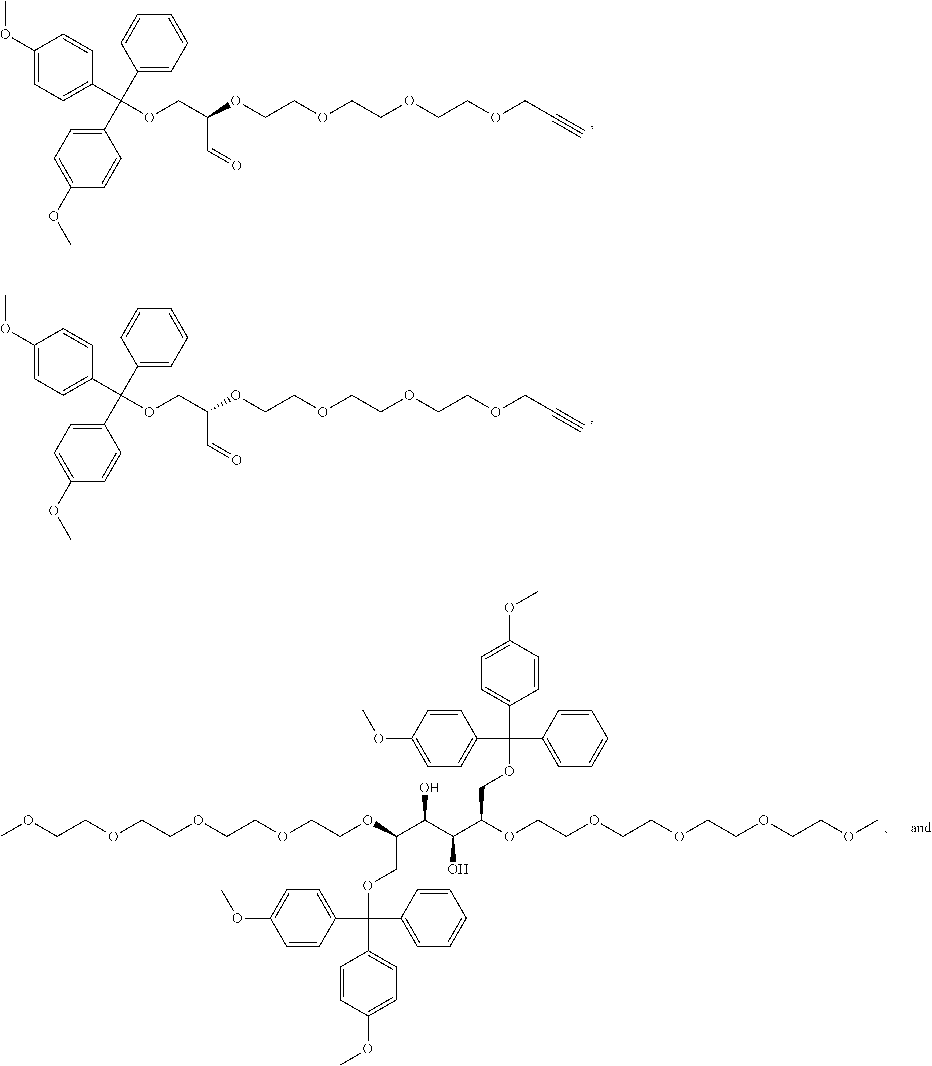

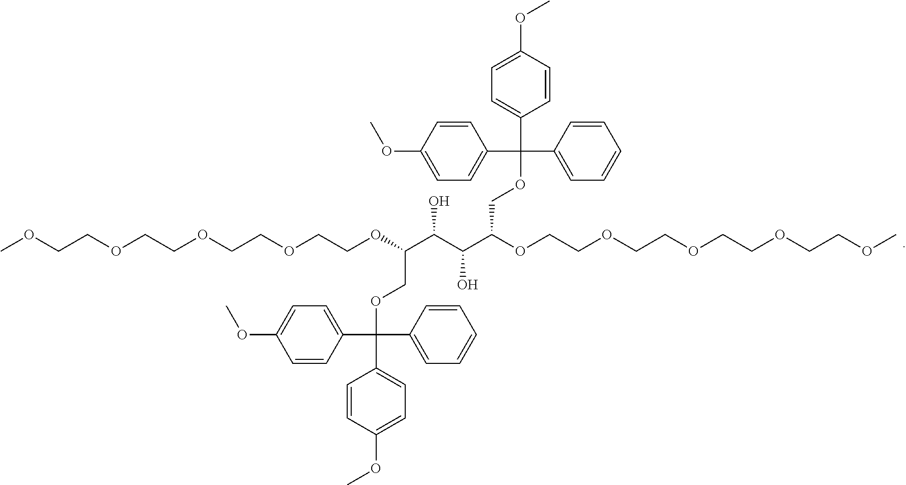

The synthesized compounds can be separated from a reaction mixture and further purified by a method such as column chromatography, high pressure liquid chromatography, precipitation, or recrystallization. Further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

### EXAMPLES

The following materials, having the abbreviations as indicated, were obtained from the mentioned sources in the United States, unless otherwise indicated. TOM-Cl (2-O-Triisopropylsilyloxymethyl) was purchased from Astatech, Inc. (Bristol, PA). NaH (sodium hydride), MeOH (methanol), toluene, THF (tetrahydrofuran), TBAF (tetrabutylammonium fluoride), DCM (dichloromethane), DMSO (dimethylsulfoxide), Na ascorbate (sodium ascorbate), sodium bicarbonate, copper sulfate, and acetic acid were obtained from Sigma-Aldrich (St. Louis, MO). DMT-Cl (4,4′-dimethoxytrityl chloride) and PPA-Cl (N,N-diisopropylamino cyanoethyl phosphonamidic chloride) from ChemGenes Corporation (Wilmington, MA). Toluene, TEA (triethylamine), hexanes, EtOAc (ethyl acetate), EDTA (ethylenediaminetetraacetic acid), diethyl ether, L-(+)-Erythrulose from EMD Millipore (Billerica, MA). m-PEG4-Tos was made from m-PEG4-OH (2,5,8,11-tetraoxatridecan-13-ol, Cat. No. BP-23742) from BroadPharm (San Diego, CA). Alkyne-PEG4-OTs was made from Alkyne-PEG4-OH (Triethylene Glycol Mono(2-propynyl) Ether, Cat. No. T3114), 1,1′-Thiocarbonyldiimidazole, L-mannitol, D-mannitol, benzaldehyde, NaIO4 (sodium periodate), NaBH4 (sodium borohydride), MBn (4-Methoxybenzyl Chloride), DDQ (2,3-Dichloro-5,6-dicyano-1,4-benzoquinone), and thiomorpholine were purchased from TCI, Inc (Portland, OR). 2,3-Isopropylidene-sn-glycerol was purchased from Biosynth Ltd. TBDPSCl, SEM-Cl, pTsOH, Ph₃P, Pd/C and Pd(OH)₂/C were purchased from Sigma-Aldrich.

High performance liquid chromatography (HPLC) was performed on a ProStar Helix™ HPLC system from Agilent Technologies, Inc. (Santa Clara, CA) consisting of two pumps (ProStar 210 Solvent Delivery Modules) with 10 ml titanium pump heads, a column oven (ProStar 510 Air Oven), a UV detector (ProStar 320 UV/Vis Detector) set at 292 nm. The system is controlled by Star Chromatography Workstation Software (version 6.41). The column used was a Cadenza Guard Column System CD-C18 (2.0 mm×5 mm) both from Imtakt USA (Portland, OR). The buffers used are Buffer A (100 mM triethylammonium acetate, pH 7.0) and Buffer B (100 mM triethylammonium acetate, pH 7.0 with 95% by volume acetonitrile). Automated solid phase phosphoramidite synthesis was done on a MerMade™ 12 synthesizer (Bioautomation Corp, Plano, TX). Synthesis solutions for the MerMade™ were purchased from Glen Research (Sterling, VA). NuMega operates 500 MHz Bruker NMR spectrometers, Avance II and AV-500 (San Diego, CA).

### Example 1: Synthesis of DMT Phosphoramidite Via the TBDPS Protecting Group Using Kinetic Resolution

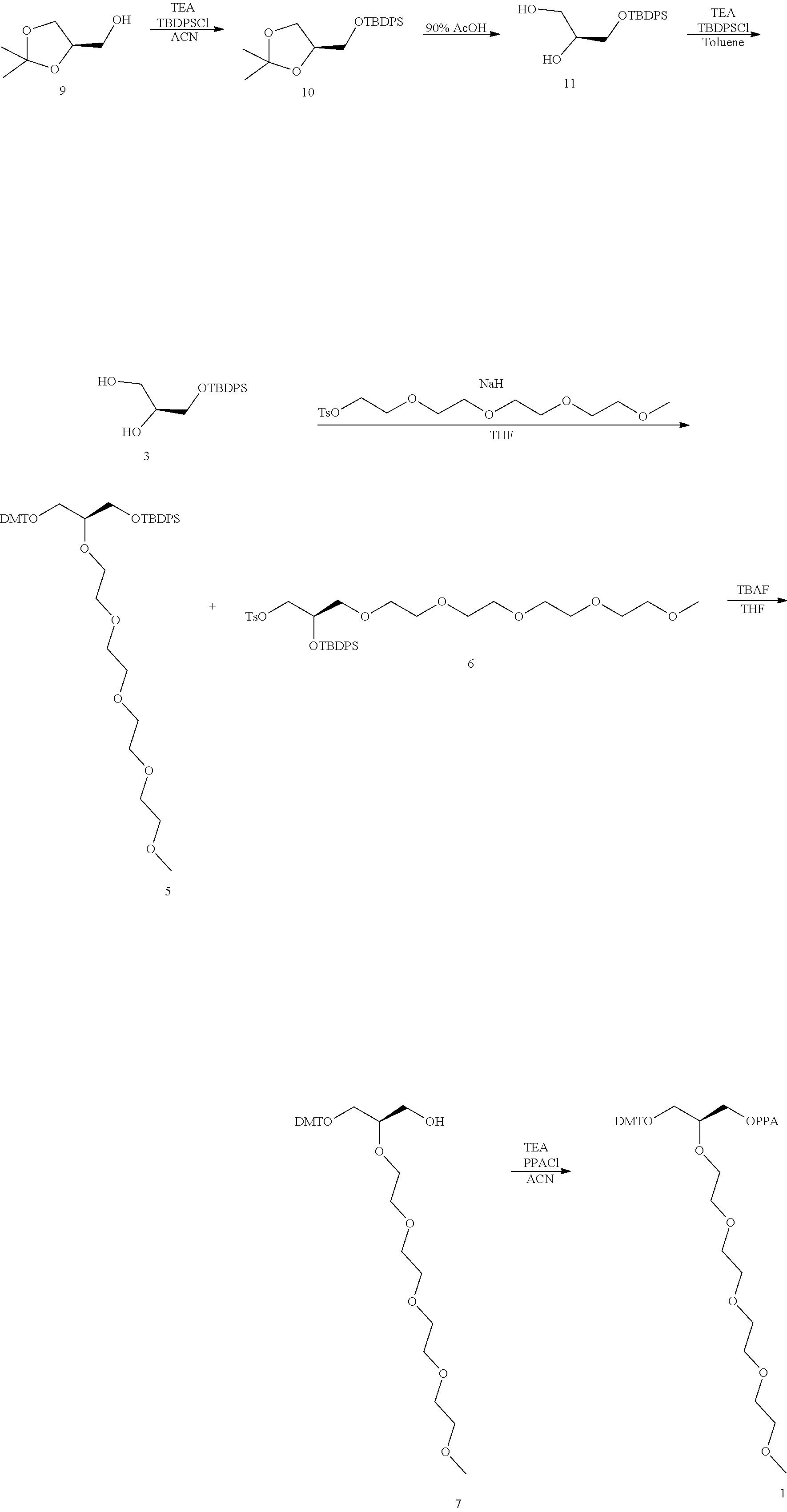

2,3-Isopropylidene-sn-glycerol 9 was dissolved in anhydrous ACN and TEA. DMAP and TBDPS-Cl were added. The solvent was evaporated, and the residue extracted from water with EtOAc and purified by flash chromatography to afford product 10 in 99% yield.

Silyl ether 10 was dissolved in 90% AcOH and heated 20 min at 80° C. The solvent was removed under reduced pressure. The residue was resuspended in EtOAc and was extracted with water and brine. The solvent was evaporated to afford diol 11 in 100% yield.

Diol 11 was dissolved in toluene and TEA. A solution of DMT-Cl in toluene was added dropwise at rt. The reaction mixture was extracted with water and brine. The solvent was evaporated, and the residue then purified by flash chromatography to afford the mono-trityl product 3 in 64% yield.

Secondary alcohol 3 was dissolved in THF, NaH (60% dispersion in mineral oil) and mPEG4Tos were added. After the reaction was completed, water was added, and the aqueous phase was extracted with EtOAc. The combined extracts were dried and concentrated in vacuo. The crude product was purified by flash chromatography to afford 5 and about 15% 6 in 77% yield.

The mixture of mPEG4 ether 5 and 6 was resuspended in THF and TBAF (1 M in THF) was added and was stirred 50 min at rt. The reaction was quenched with sat. solution of NaHCO₃ and the aqueous phase was extracted with EtOAc. The combined extracts were dried and concentrated in vacuo. The crude product was purified by flash chromatography to afford 7 in 80% yield.

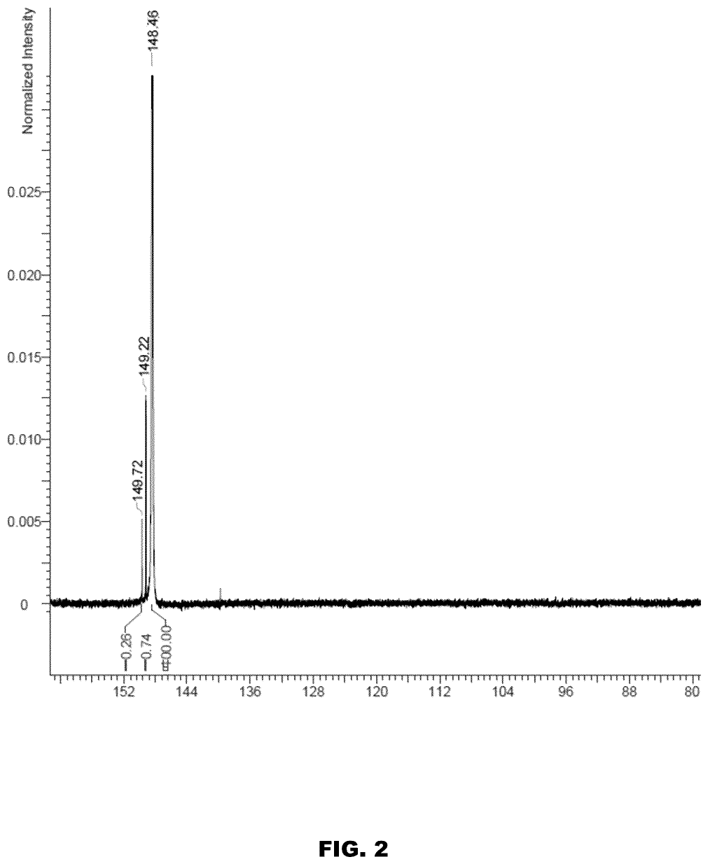

DMT PEG4 alcohol 7 was dissolved in ACN and TEA. PPA-Cl was added, and the reaction was stirred 1 h at rt. The precipitate was filtered off and the solvent dried down under reduced pressure. The crude product was purified by flash chromatography to afford 1 in 70% yield. TLC (EtOAc+0.5% TEA): Rf=0.75, 1H NMR (500 MHz, ACETONITRILE-d3) δ ppm 1.04-1.22 (m, 12H), 2.49-2.62 (m, 2H), 3.05-3.19 (m, 2H), 3.22-3.28 (m, 3H), 3.44 (br dd, J=5.94, 3.17 Hz, 3H), 3.48-3.58 (m, 14H), 3.60-3.76 (m, 13H), 6.81-6.87 (m, 4H), 7.16-7.25 (m, 1H), 7.26-7.34 (m, 6H), 7.41-7.48 (m, 2H), 31P NMR (500 MHz, ACETONITRILE-d3) δ ppm 148.43, 148.47, 149.23, 149.72 (see FIG. 2).

### Example 2: Synthesis of DMT Phosphoramidite 1 Via the TOM Protecting Group

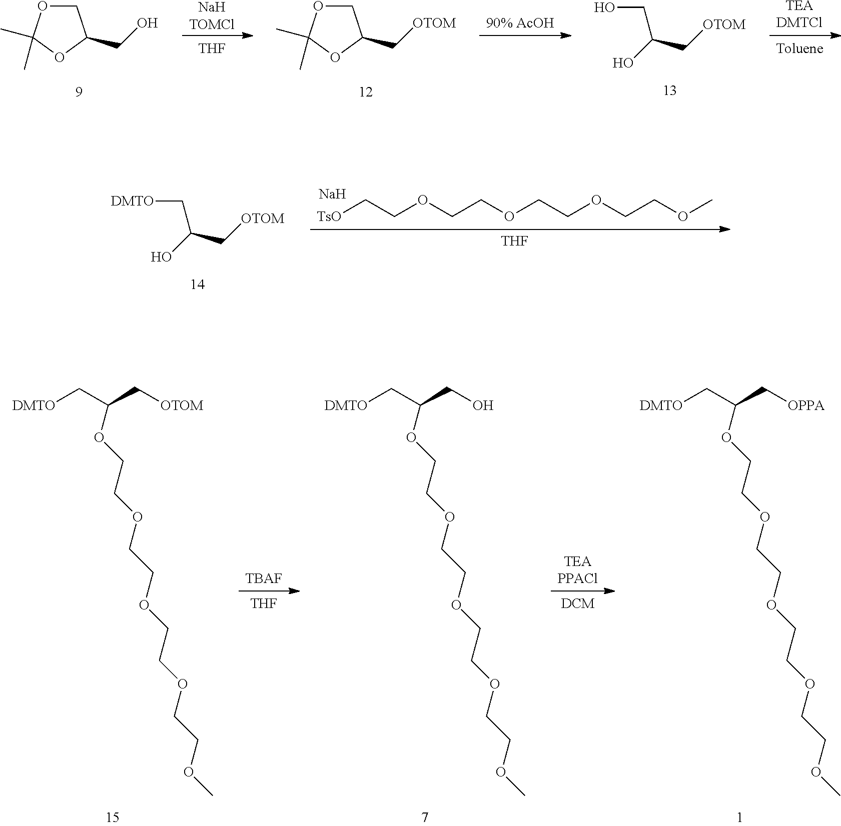

2,3-Isopropylidene-sn-glycerol 9 was dissolved in anhydrous THF, NaH (60% dispersion in mineral oil) and TOMCl were added. After the reaction was completed, water was added, and the aqueous phase was extracted with EtOAc. The combined extracts were dried and concentrated in vacuo. The crude product was purified by flash chromatography to afford 12 in 49% yield.

TOM-protected ether 12 was dissolved in 90% AcOH and heated to 80° C. for 30 min. The reaction mixture was concentrated in vacuo and the crude product was purified by flash chromatography to afford diol 13 in 80% yield.

Diol 13 was dissolved in toluene, TEA and DMTCl were added, and the reaction mixture was stirred at rt. After the reaction was completed, water was added, and the aqueous phase was extracted with toluene. The combined extracts were dried and concentrated in vacuo. The crude product was purified by flash chromatography to afford 14 in 59% yield.

Secondary alcohol 14 was dissolved in THF, NaH (60% dispersion in mineral oil) and mPEG4Tos were added. After the reaction was completed, water was added, and the aqueous phase was extracted with EtOAc. The combined extracts were dried and concentrated in vacuo. The crude product was purified by flash chromatography to afford 15 in 82% yield.

mPEG4 ether 15 was dissolved in THF, TBAF (1 M in THF) was added, and the reaction mixture was stirred at rt. After the reaction was completed, sat. solution of NaHCO₃ was added and the aqueous phase was extracted with EtOAc. The combined extracts were dried and concentrated in vacuo. The crude product was purified by flash chromatography to afford 7 in 71% yield.

DMT PEG4 alcohol 7 was dissolved in DCM, TEA and PPACl were added, and the reaction mixture was stirred at rt. After the reaction was completed, water was added, and the aqueous phase was extracted with DCM. The combined extracts were dried and concentrated in vacuo. The crude product was purified by flash chromatography to afford 1 in 67% yield. The structure of the phosphoramidite 1 was confirmed by ¹H and ³¹P NMR (CD₃CN). No regioisomers were detected.

### Example 3: Synthesis of DMT Phosphoramidite Via the Azidomethyl Protecting

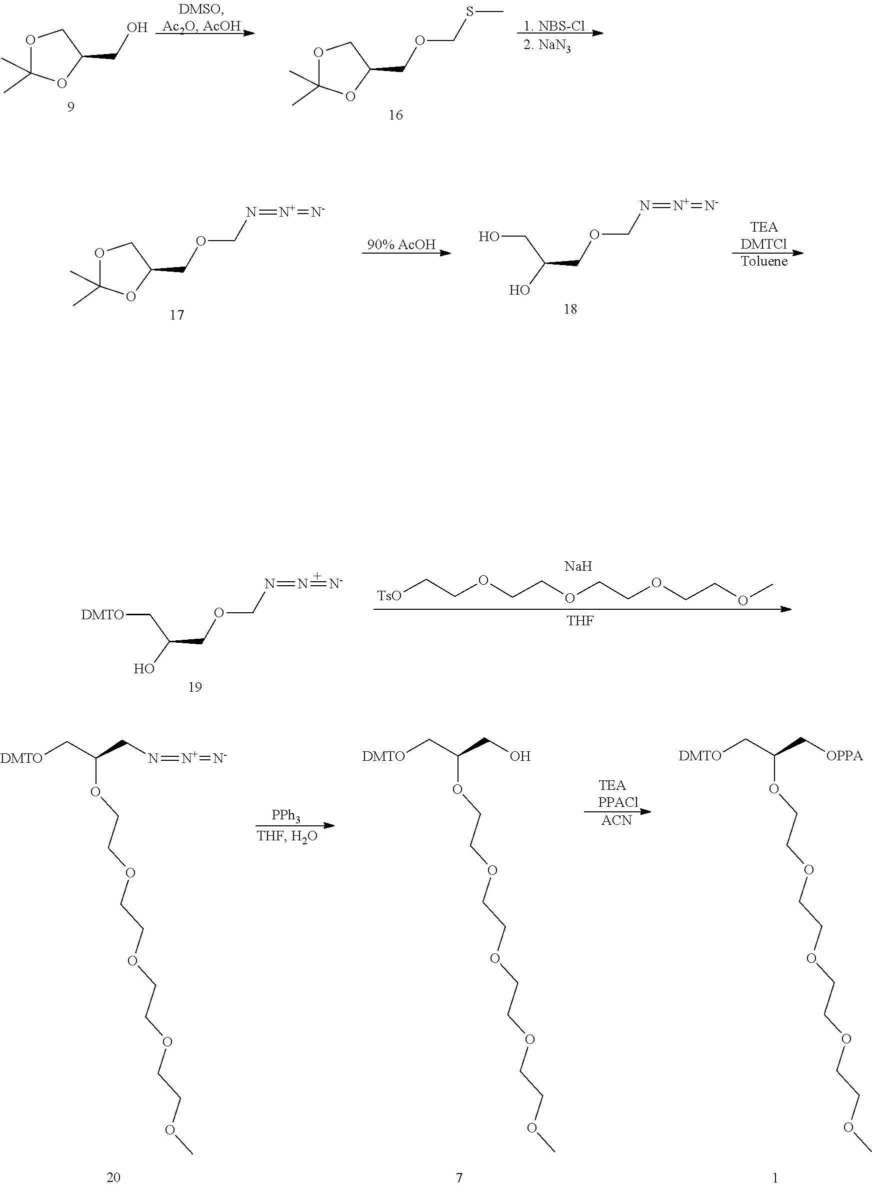

The azidomethyl group was introduced in the two following steps based on literature methods: (1) PNAS 2008, 105(27), 9145-9150; (2) Russian Journal of Bioorganic Chemistry 2009, 35, 270-273.

2,3-Isopropylidene-sn-glycerol 9 was dissolved in DMSO, AcOH and Ac₂O. The reaction mixture was stirred 50 h at rt. The reaction was quenched with sat. solution of NaHCO₃ and the aqueous phase was extracted with EtOAc. The EtOAc was evaporated, and the residue dried under vacuum to afford 16 as crude product in 63% yield.

The crude thio ether 16 was solved in DMF and 2-nitrobenzene sulfonyl chloride was added. After 5 min the reaction mixture was cooled to 0° C. and NaN₃ was added. After 1 h the reaction was quenched with half concentrated NaHCO₃ solution, and the aqueous phase was extracted with EtOAc. The crude product was purified by flash chromatography to afford 17 in 58% yield.

Azidomethyl ether 17 was dissolved in 90% AcOH and heated 20 min at 80° C. The solvent was removed under reduced pressure. The residue was resuspended in EtOAc and was extracted with water and brine. The solvent was evaporated to afford diol 18 in 87% yield.

Diol 18 was dissolved in toluene and TEA. A solution of DMT-Cl in toluene was added dropwise at rt. The reaction mixture was extracted with water and brine. The solvent was evaporated, and the residue then purified by flash chromatography to afford the mono-trityl product 19 in 88% yield.

Secondary alcohol 19 was dissolved in THF, NaH (60% dispersion in mineral oil) and mPEG4Tos was added. After the reaction was completed, water was added, and the aqueous phase was extracted with EtOAc. The combined extracts were dried and concentrated in vacuo. The crude product was purified by flash chromatography to afford 20 in 70% yield.

mPEG4 ether 20 was dissolved in THF, PPh₃ and H₂O were added. The reaction mixture was stirred 18 h at rt. The solvent was evaporated, and the crude product was purified by flash chromatography to afford 7 in 90% yield.

DMT PEG4 alcohol 7 was dissolved in ACN and TEA. PPA-Cl was added, and the reaction was stirred 1 h at rt. The precipitate was filtered off and the solvent dried down under reduced pressure. The crude product was purified by flash chromatography to afford 1 in 49% yield. The structure of the phosphoramidite 1 was confirmed by ¹H and ³¹P NMR (CD₃CN). No regioisomers were detected.

### Example 4: Synthesis of DMT Phosphoramidite Via the Allyl Protecting Group

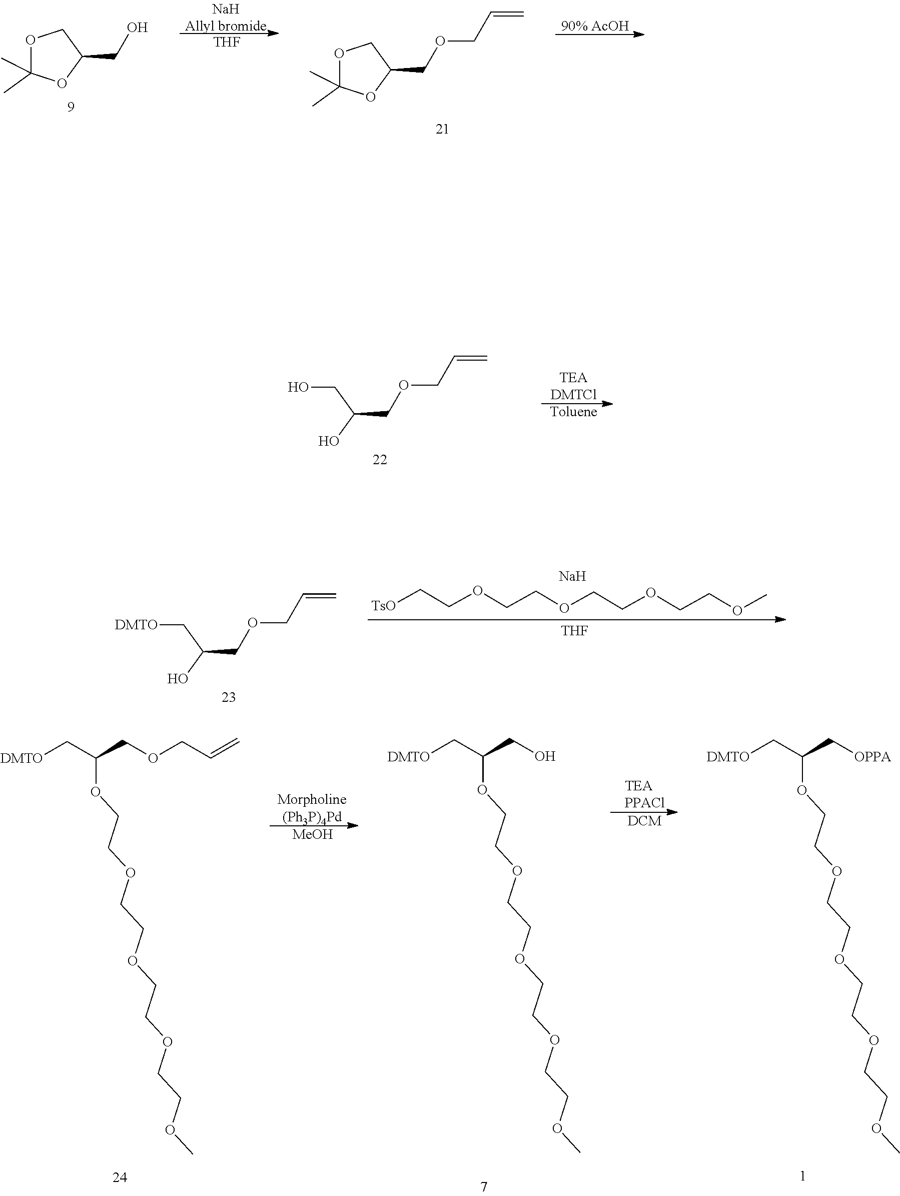

2,3-Isopropylidene-sn-glycerol 9 was added to an ice-cold suspension of NaH (60% dispersion in mineral oil) in anhydrous THF. Allyl bromide was added, and the reaction mixture was warmed to rt. After the reaction was completed, water was added, and the aqueous phase was extracted with EtOAc. The combined extracts were washed with water and brine. Organic phase was dried and concentrated in vacuo. The crude product was solved in 90% AcOH and heated to 80° C. for 2 h. The reaction mixture was concentrated in vacuo and the crude product was purified by flash chromatography to afford diol 22 in 63% yield.

Diol 22 was dissolved in toluene, TEA and DMTCl were added, and the reaction mixture was stirred at rt. After the reaction was completed, water was added, and the aqueous phase was extracted with toluene. The combined extracts were dried and concentrated in vacuo. The crude product was purified by flash chromatography to afford 23 in 79% yield.

Secondary alcohol 23 was dissolved in THF, NaH (60% dispersion in mineral oil) and mPEG4Tos were added. After the reaction was completed, water was added, and the aqueous phase was extracted with EtOAc. The combined extracts were dried and concentrated in vacuo. The crude product was purified by flash chromatography to afford 24 in 90% yield.

mPEG4 ether 24 was dissolved in MeOH, morpholine and (Ph₃P)₄Pd were added and the reaction mixture was stirred in the microwave at 90° C. for 4 h. Catalyst was filtered off, the filtrate was concentrated in vacuo and the crude product was purified by flash chromatography to afford 7 in 63% yield.

DMT PEG4 alcohol 7 was dissolved in DCM, TEA and PPACl were added, and the reaction mixture was stirred at rt. After the reaction was completed, water was added, and the aqueous phase was extracted with DCM. The combined extracts were dried and concentrated in vacuo. The crude product was purified by flash chromatography to afford 1 in 67% yield. The structure of the phosphoramidite 1 was confirmed by 1H and 31P NMR (CD3CN). No regioisomers were detected.

### Example 5: Synthesis of DMT Phosphoramidite Via the SEM Protecting Group

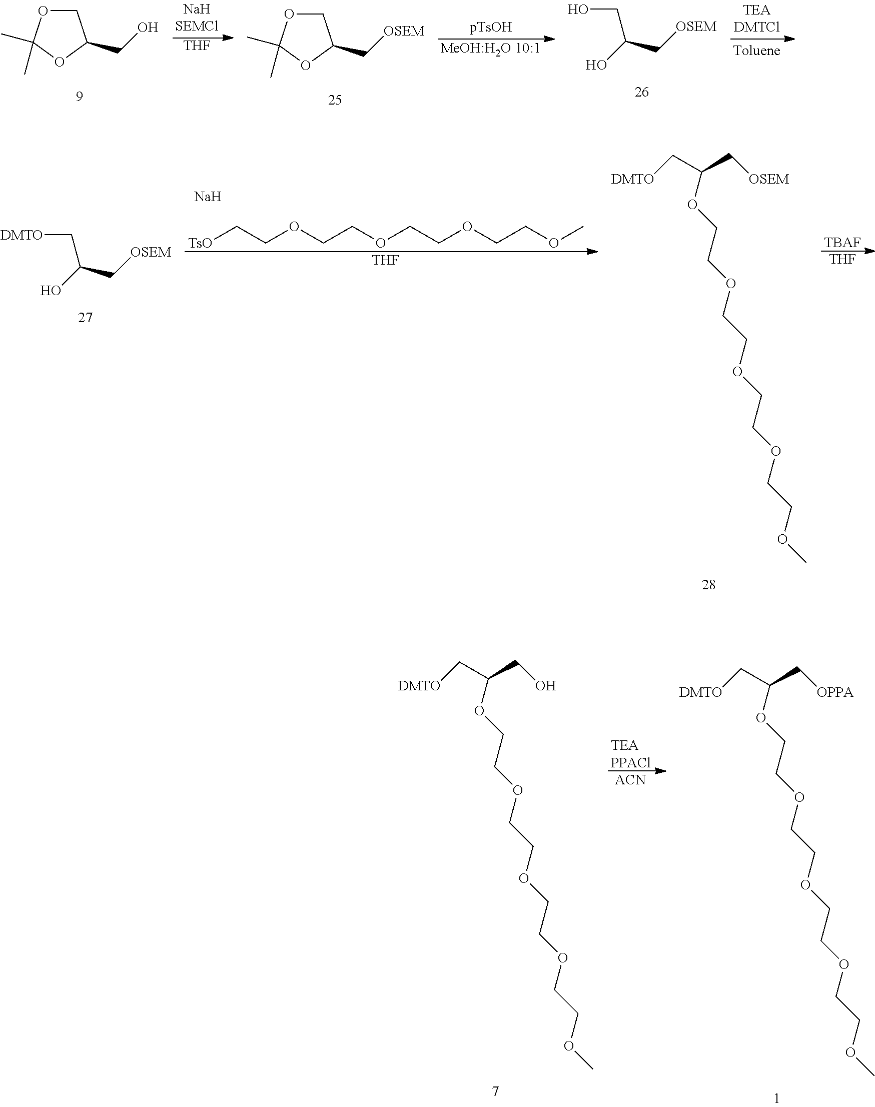

Compound 26 was prepared according to Bull. Soc. Chem. Jpn. 1987, 60, 2169-2172.

2,3-Isopropylidene-sn-glycerol 9 was added to an ice-cold suspension of NaH (60% dispersion in mineral oil) in THF. SEMCl was added and the reaction mixture was stirred in an ice bath. After the reaction was completed, water was added, and the aqueous phase was extracted with EtOAc. The combined extracts were dried and concentrated in vacuo. The crude product was purified by flash chromatography to afford 25.

SEM-protected ether 25 was dissolved in 90% AcOH and heated to 80° C. for 30 min. The reaction mixture was concentrated in vacuo and the crude product was purified by flash chromatography to afford diol 26.

Diol 26 was dissolved in toluene, TEA and DMTCl were added, and the reaction mixture was stirred at rt. After the reaction was completed, water was added, and the aqueous phase was extracted with toluene. The combined extracts were dried and concentrated in vacuo. The crude product was purified by flash chromatography to afford 27.

Secondary alcohol 27 was dissolved in THF, NaH (60% dispersion in mineral oil) and mPEG4Tos were added. After the reaction was completed, water was added, and the aqueous phase was extracted with EtOAc. The combined extracts were dried and concentrated in vacuo. The crude product was purified by flash chromatography to afford 28.

Compound 28 was dissolved in THF, TBAF (1 M in THF) was added, and the reaction mixture was stirred at 100° C. in the microwave. After the reaction was completed, sat. solution of NaHCO₃ was added and the aqueous phase was extracted with EtOAc. The combined extracts were dried and concentrated in vacuo. The crude product was purified by flash chromatography to afford 7.

DMT PEG4 alcohol 7 was dissolved in ACN, TEA and PPACl were added, and the reaction mixture was stirred at rt. After the reaction was completed, water was added, and the aqueous phase was extracted with DCM. The combined extracts were dried and concentrated in vacuo. The crude product was purified by flash chromatography to afford 1.

### Example 6: Synthesis of DMT Phosphoramidite 1b Via the TOM Protecting Group

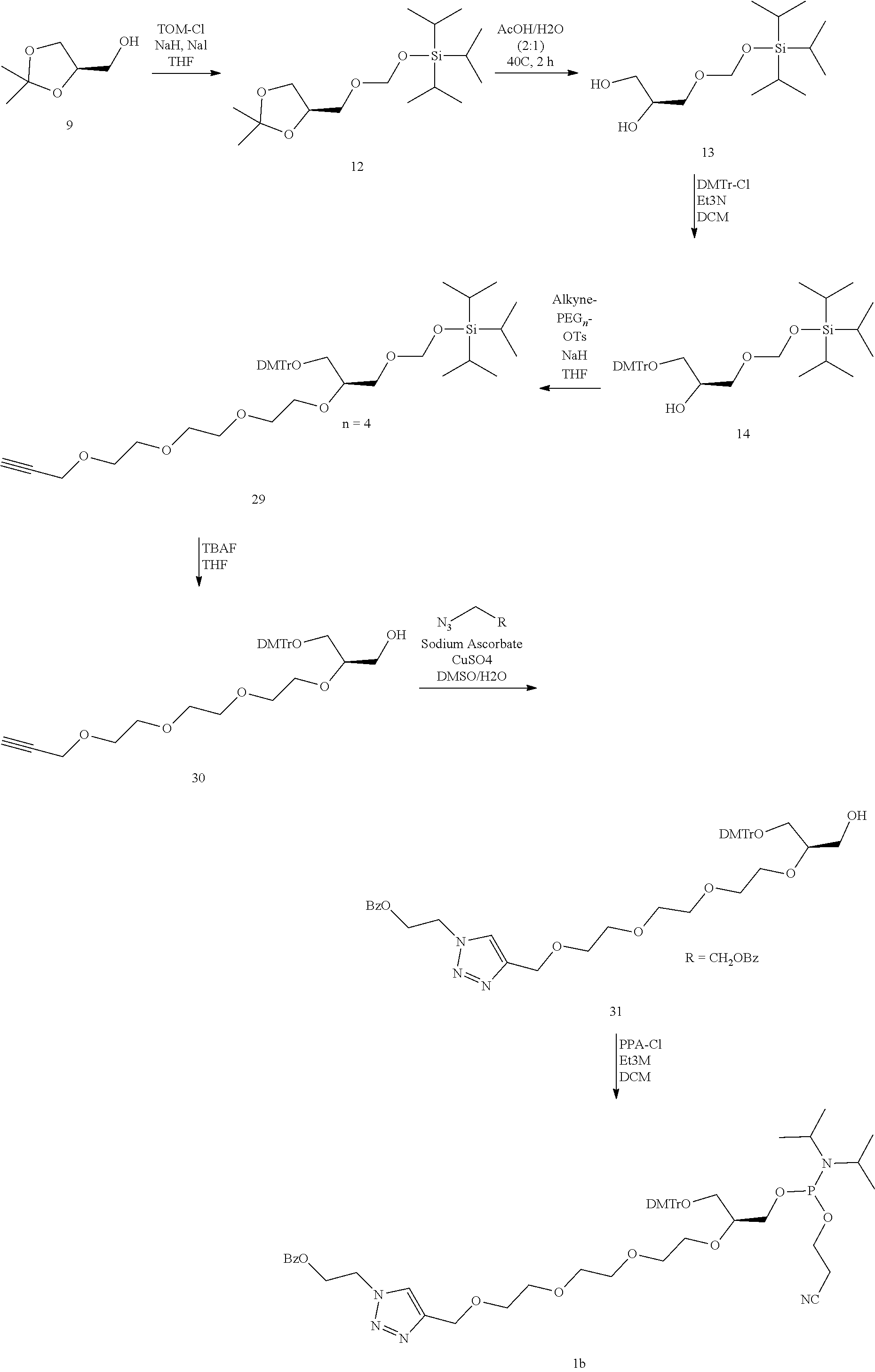

2,3-Isopropylidene-sn-glycerol 9 was dissolved in anhydrous THF. Sodium hydride was added to generate alkoxide. TOM-Cl was dissolved in THF, charged with NaI, and added portion-wise. The reaction was incubated for 24 hours. Excess NaH was quenched with MeOH, then diluted with water and extracted with EtOAc. The combined organic layers were concentrated under reduced pressure. Oils were separated by extraction with ACN. The residue was purified by flash chromatography to afford protected 12.

Product 12 was dissolved in a mixture of acetic acid and water (2:1). The solution was heated to 40 C and stirred for 120 minutes, then neutralized with sodium bicarbonate and extracted with EtOAc. Organics were separated, dried over Na₂SO₄, and concentrated under reduced pressure. The residue was purified by flash chromatography to afford diol 13.

Diol 13 was dissolved in DCM and TEA. A solution of DMT-Cl in DCM was added portion-wise. MeOH was added and the reaction was dried under reduced pressure. The residue was resuspended in toluene and separated from the salts, then purified by flash chromatography to afford the mono-trityl product 14.

1-O-TOM-3-O-DMTr-propane-1,2,3-triol 14 (from Example 2) was dissolved in anhydrous THF. Sodium hydride was added to generate alkoxide. When the bubbling ceased, alkyne tosylate (prepared via tosylation of Cat. No. BP-21657, Broadpharm) was added portion-wise. The reaction was incubated with stirring for 24-48 h. Excess NaH was quenched with water, then the solution was transferred to a separatory funnel and extracted with ethyl acetate. The combined organic layers were washed with brine, dried with sodium sulfate, filtered, and concentrated under reduced pressure. The residue was resuspended in toluene, separated from remaining salts, and purified by flash chromatography to afford Alkyne 29.

Alkyne 29 was resuspended in THF and TBAF was added and stirred for 2 hrs. The reaction was concentrated under reduced pressure and purified by flash chromatography to afford 30.

To a solution of alkyne 30 in DMSO was added 2-azidoethyl acetate. In a separate vial, sodium ascorbate was dissolved in water/DMSO followed by 1M CuSO4, to prepare the catalyst mixture. The catalyst mixture was added to a solution of alkyne/azide dropwise over 10 minutes. Upon completion, reaction was quenched with 0.5M EDTA and stirred for 15 minutes. Dilute with water and extract with ethyl acetate three times. Wash combined organics with brine and dried over sodium sulfate. The residue was resuspended in toluene and purified by flash chromatography to afford triazole 31.

Alcohol 31 was dissolved in DCM and TEA under an inert atmosphere. PPA-Cl was added, and the reaction was stirred for 60 minutes. The reaction was dried down under reduced pressure and resuspended in toluene with 0.5% TEA, then purified by flash chromatography (Silica gel basified by 0.5% TEA mobile phase). Phosphoramidite 1b was isolated and regiopurity was confirmed by 1H and 31P NMR.

### Example 7: Synthesis of DMT Phosphoramidite 1 Via the TC Protecting Group

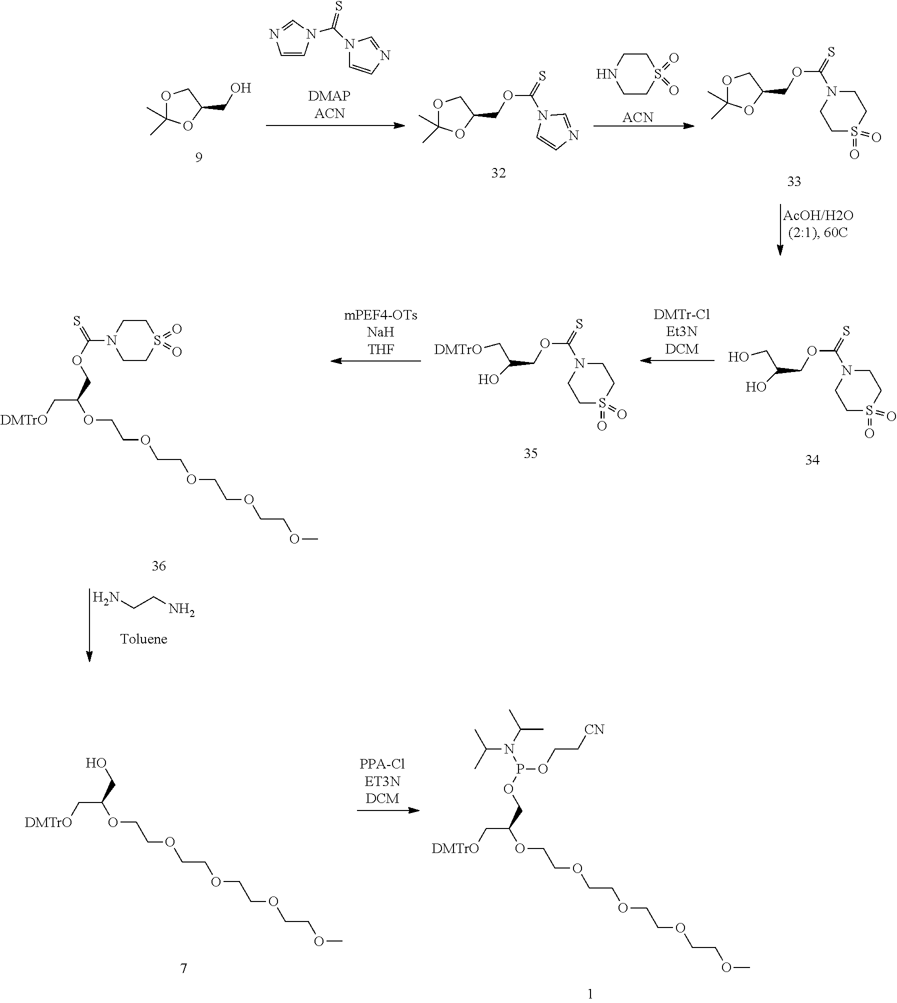

2,3-Isopropylidene-sn-glycerol 9 was dissolved in anhydrous ACN. The reaction mixture was charged portion-wise with thiocarbonyldiimidazole, then DMAP portion-wise and stirred for 4 hours. The reaction mixture was concentrated under reduced pressure. The crude residue was purified by flash chromatography to afford 32.

Thiobocarbamate glycerol 32 was dissolved in anhydrous ACN and charged with thiomorpholine portion-wise. The reaction mixture stirred under an inert atmosphere overnight then concentrated under reduced pressure. The rude residue was purified by flash chromatography to afford 2′-Thiomorpholine-4-carbothioate 33.

Product 33 was dissolved in a mixture of acetic acid and water (2:1). The solution was heated to 40° C. and stirred for 120 minutes, then neutralized with sodium bicarbonate and extracted with EtOAc. Organics were separated, dried over Na₂SO₄, and concentrated under reduced pressure. The residue was purified by flash chromatography to afford diol 34.

Diol 34 was dissolved in DCM and TEA. A solution of DMT-Cl in DCM was added portion-wise. MeOH was added and the reaction was dried under reduced pressure. The residue was resuspended in toluene and separated from the salts, then purified by flash chromatography to afford 35.

Secondary alcohol 35 was dissolved in anhydrous THF. Sodium hydride was added to generate alkoxide. When the bubbling ceased, mPEGn-OTs was dissolved in THF and added portion-wise. The reaction stirred vigorously at ambient temperatures overnight. Excess NaH was quenched with water, then diluted with water and extracted with EtOAc. The combined organic layers were dried under reduced pressure. The residue was resuspended in toluene, separated from remaining salts, and purified by flash chromatography to afford 36.

2′-Thiomorpholine-4-carbothioate 36 was resuspended in Toluene and Ethylenediamine (1:1) under an inert atmosphere. The reaction was stirred overnight and then concentrated under reduced pressure. The residue was purified by flash chromatography to afford free alcohol 7.

Alcohol 7 was dissolved in DCM and TEA under an inert atmosphere. PPA-Cl was added, and the reaction was stirred for 60 minutes. The reaction was dried down under reduced pressure and resuspended in toluene with 0.5% TEA, then purified by flash chromatography (Silica gel basified by 0.5% TEA mobile phase) to afford phosphoramidite 1.

### Example 8: Synthesis of DMT Phosphoramidite 1b Via the MBn Protecting Group

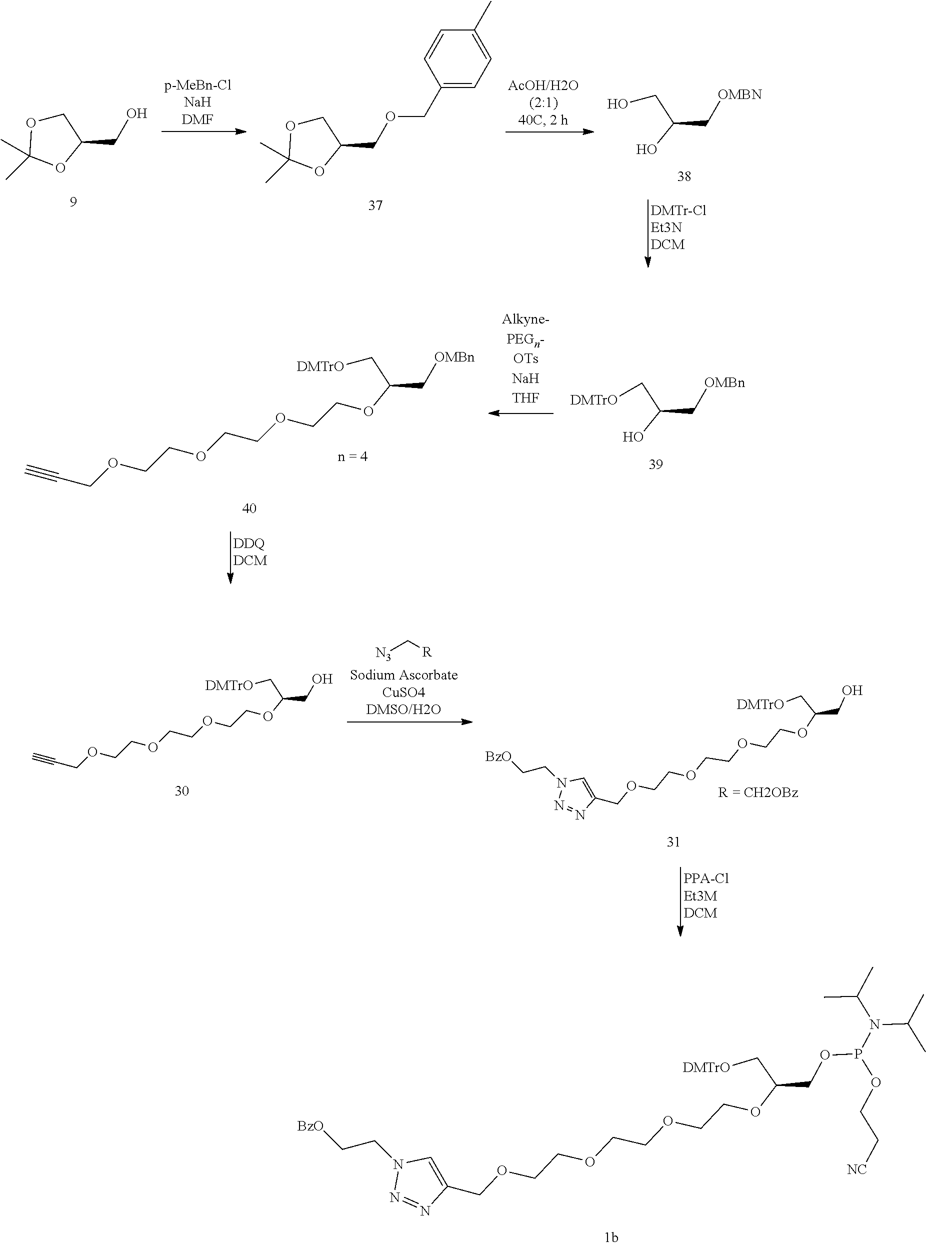

2,3-Isopropylidene-sn-glycerol 9 was dissolved in anhydrous DMF. The reaction mixture was charged portion-wise with NaH to form the alcoxide, then para-methylbenzyl chloride (MBn-Cl) and stirred for 4 hours. The reaction mixture was concentrated under reduced pressure. The crude residue was purified by flash chromatography to afford 37.

Product 37 was dissolved in a mixture of Acetic acid and water (2:1). The solution was heated to 40 C and stirred for 120 minutes, then neutralized with sodium bicarbonate and extracted with EtOAc. Organics were separated, dried over Na₂SO₄, and concentrated under reduced pressure. The residue was purified by flash chromatography to afford diol 38.

Diol 38 was dissolved in DCM and TEA. A solution of DMT-Cl in DCM was added portion-wise. MeOH was added and the reaction was dried under reduced pressure. The residue was resuspended in toluene and separated from the salts, then purified by flash chromatography to afford mono-trityl product 39.

Alcohol 39 was dissolved in anhydrous THF. Sodium hydride was added to generate alkoxide. When the bubbling ceased, alkyne tosylate (prepared via tosylation of Cat. No. BP-21657, Broadpharm) was added portion-wise. The reaction was incubated with stirring for 24-48 h. Excess NaH was quenched with water, then the solution was transferred to a separatory funnel and extracted with ethyl acetate. The combined organic layers were washed with brine, dried with sodium sulfate, filtered, and concentrated under reduced pressure. The residue was resuspended in toluene, separated from remaining salts, and purified by flash chromatography to afford Alkyne 40.

40 was resuspended in DCM and DDQ was added and stirred for 60 minutes. The reaction was concentrated under reduced pressure and purified by flash chromatography to afford 30.

To a solution of alkyne 30 in DMSO was added 2-azidoethyl acetate. In a separate vial, sodium ascorbate was dissolved in water/DMSO followed by 1M CuSO4, to prepare the catalyst mixture. The catalyst mixture was added to a solution of alkyne/azide dropwise over 10 minutes. Upon completion, reaction was quenched with 0.5M EDTA and stirred for 15 minutes. Dilute with water and extract with ethyl acetate three times. Wash combined organics with brine and dried over sodium sulfate. The residue was resuspended in toluene and purified by flash chromatography to afford triazole 31.

Alcohol 31 was dissolved in DCM and TEA under an inert atmosphere. PPA-Cl was added, and the reaction was stirred for 60 minutes. The reaction was dried down under reduced pressure and resuspended in toluene with 0.5% TEA, then purified by flash chromatography (Silica gel basified by 0.5% TEA mobile phase). Phosphoramidite 1b was isolated and regiopurity was confirmed by 31P NMR.

### Example 9: Synthesis of DMT Phosphoramidites Via the Mannitol

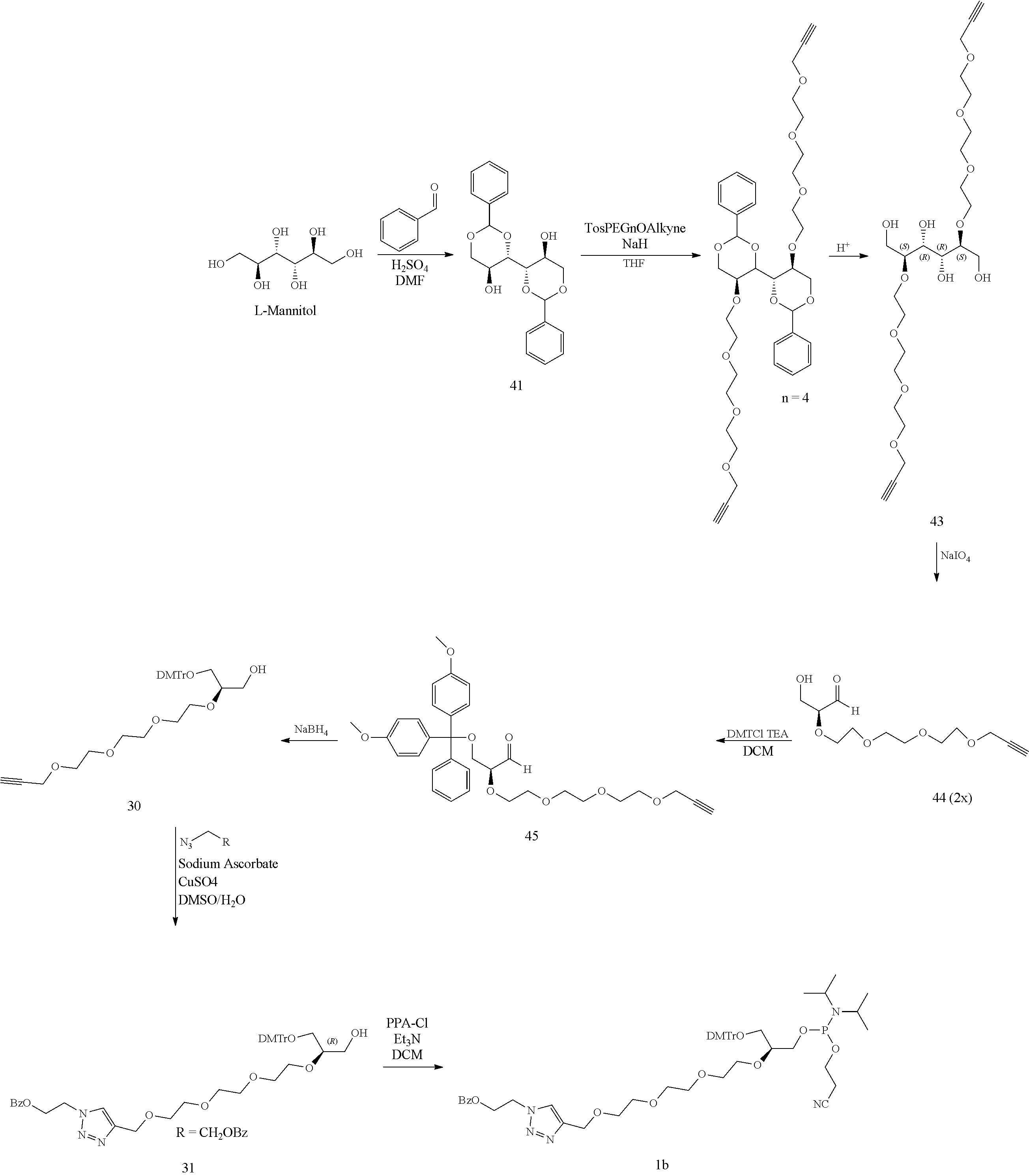

L-mannitol was dissolved in DMF then slowly charged with sulfuric acid and dropwise addition of benzaldehyde at OC. Reaction equilibrated to ambient temperatures and stirred until completion by TLC. Excess sulfuric acid was quenched with sodium bicarbonate and extracted with EtOAc. Organics were separated, dried over Na₂SO₄, and concentrated under reduced pressure. The residue was purified by flash chromatography to afford the di-acetal product 41.

Corresponding diol 41 was dissolved in anhydrous THF. Sodium hydride was added to generate alkoxide. When the bubbling ceased, alkyne tosylate (prepared via tosylation of Cat. No. BP-21657, Broadpharm) was added portion-wise. The reaction was incubated with stirring for 24-48 h. Excess NaH was quenched with water, then the solution was transferred to a separatory funnel and extracted with ethyl acetate. The combined organic layers were washed with brine, dried with sodium sulfate, filtered, and concentrated under reduced pressure. The residue was resuspended in toluene, separated from remaining salts, and purified by flash chromatography to afford bis-Alkyne 42.

42 was dissolved in MeOH and HCl was added. The solution was incubated for 20 minutes, then neutralized with sodium bicarbonate and dried under reduced pressure. The residue was resuspended in ethyl acetate and purified by flash chromatography to afford tetraol 43.

43 was charged with sodium periodate in water and incubated at room temperature for 2 hours. The solution was transferred to a separatory funnel and extracted with ethyl acetate. The combined organic layers were washed with brine, dried with sodium sulfate, filtered, and concentrated under reduced pressure. The residue was resuspended in toluene, separated from remaining salts, and purified by flash chromatography to afford two molar equivalents of aldehyde 44.

Free alcohol 44 was dissolved in DCM and TEA. A solution of DMT-Cl in DCM was added portion-wise and stirred for 2 hours. MeOH was added and the reaction was dried under reduced pressure. The residue was resuspended in toluene and separated from the salts, then purified by flash chromatography to afford the mono-trityl product 45.

45 was dissolved in MeOH and cooled to 0 C. Sodium borohydride (NaBH4) was added portion-wise under an inert atmosphere and stirred for 60 minutes. Water was added and extracted with EtOAc. The combined organic layers were washed with brine, dried with sodium sulfate, filtered, and concentrated under reduced pressure. The residue was purified by flash chromatography to afford alcohol 30.

To a solution of alkyne 30 in DMSO was added 2-azidoethyl acetate. In a separate vial, sodium ascorbate was dissolved in water/DMSO followed by 1M CuSO4, to prepare the catalyst mixture. The catalyst mixture was added to a solution of alkyne/azide dropwise over 10 minutes. Upon completion, reaction was quenched with 0.5M EDTA and stirred for 15 minutes. Dilute with water and extract with ethyl acetate three times. Wash combined organics with brine and dried over sodium sulfate. The residue was resuspended in toluene and purified by flash chromatography to afford triazole 31.

Alcohol 31 was dissolved in DCM and TEA under an inert atmosphere. PPA-Cl was added, and the reaction was stirred for 60 minutes. The reaction was dried down under reduced pressure and resuspended in toluene with 0.5% TEA, then purified by flash chromatography (Silica gel basified by 0.5% TEA mobile phase). Phosphoramidite 1b was isolated and regiopurity was established by 31P NMR.

### Example 10: Synthesis of DMT Phosphoramidites Via the Erythrulose

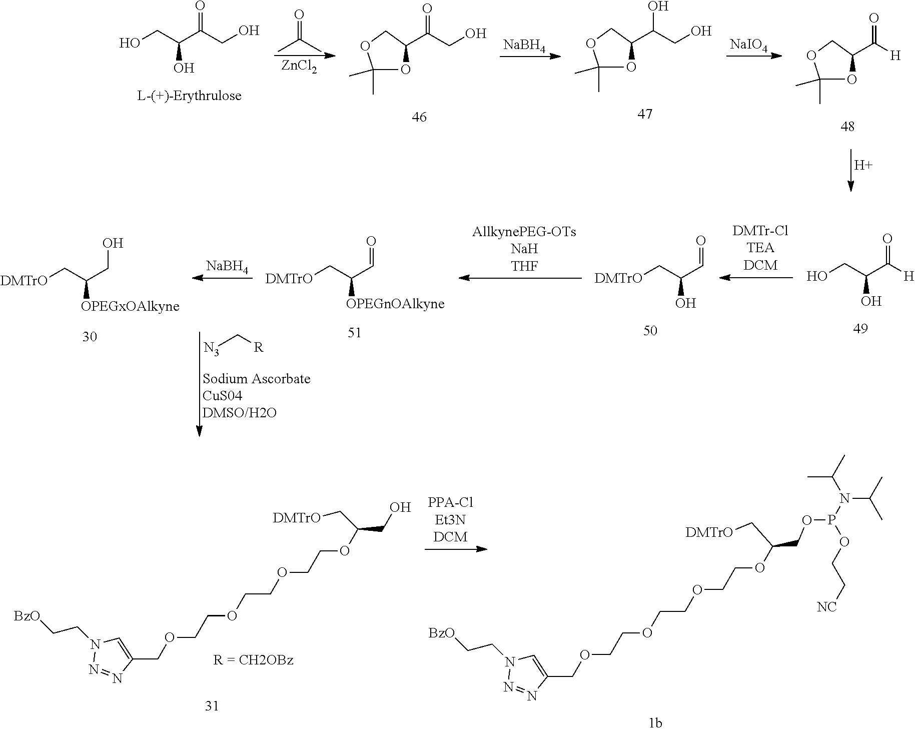

L-(+)-Erythrulose was dissolved in acetone and charged with zinc chloride and stirred under an inert atmosphere. The reaction mixture was concentrated under reduced pressure. The crude residue was purified by flash chromatography to afford acetonide 46.

46 was dissolved in MeOH and cooled to 0° C. Sodium borohydride (NaBH4) was added portion-wise under an inert atmosphere and stirred for 60 minutes. Water was added and extracted with EtOAc. The combined organic layers were washed with brine, dried with sodium sulfate, filtered, and concentrated under reduced pressure. The residue was purified by flash chromatography to afford alcohol 47.

47 was charged with sodium periodate in water and incubated at room temperature for 2 hours. The solution was transferred to a separatory funnel and extracted with ethyl acetate. The combined organic layers were washed with brine, dried with sodium sulfate, filtered, and concentrated under reduced pressure. The residue was resuspended in toluene, separated from remaining salts, and purified by flash chromatography to afford aldehyde 48.

48 was dissolved in MeOH and HCl was added. The solution was incubated for 20 minutes, then neutralized with sodium bicarbonate and dried under reduced pressure. The residue was resuspended in ethyl acetate and purified by flash chromatography to afford diol 49.

49 was dissolved in DCM and TEA. A solution of DMT-Cl in DCM was added portion-wise and stirred for 2 hours. MeOH was added and the reaction was dried under reduced pressure. The residue was resuspended in toluene and separated from the salts, then purified by flash chromatography to afford the mono-trityl product 50.

Alcohol 50 was dissolved in anhydrous THF. Sodium hydride was added to generate alkoxide. When the bubbling ceased, alkyne tosylate (prepared via tosylation of Cat. No. BP-21657, Broadpharm) was added portion-wise. The reaction was incubated with stirring for 24-48 h. Excess NaH was quenched with water, then the solution was transferred to a separatory funnel and extracted with ethyl acetate. The combined organic layers were washed with brine, dried with sodium sulfate, filtered, and concentrated under reduced pressure. The residue was resuspended in toluene, separated from remaining salts, and purified by flash chromatography to afford Alkyne 51.

51 was dissolved in MeOH and cooled to 0° C. Sodium borohydride (NaBH4) was added portion-wise under an inert atmosphere and stirred for 60 minutes. Water was added and extracted with EtOAc. The combined organic layers were washed with brine, dried with sodium sulfate, filtered, and concentrated under reduced pressure. The residue was purified by flash chromatography to afford alcohol 30.

To a solution of alkyne 30 in DMSO was added 2-azidoethyl acetate. In a separate vial, sodium ascorbate was dissolved in water/DMSO followed by 1M CuSO4, to prepare the catalyst mixture. The catalyst mixture was added to a solution of alkyne/azide dropwise over 10 minutes. Upon completion, reaction was quenched with 0.5M EDTA and stirred for 15 minutes. Dilute with water and extract with ethyl acetate three times. Wash combined organics with brine and dried over sodium sulfate. The residue was resuspended in toluene and purified by flash chromatography to afford triazole 31.

Alcohol 31 was dissolved in DCM and TEA under an inert atmosphere. PPA-Cl was added, and the reaction was stirred for 60 minutes. The reaction was dried down under reduced pressure and resuspended in toluene with 0.5% TEA, then purified by flash chromatography (Silica gel basified with a 0.5% TEA mobile phase). Phosphoramidite 1b was isolated and regiopurity was established by 31P NMR.

### Example 11: Synthesis of DMT Phosphoramidites Via the Dihydroxypropanal

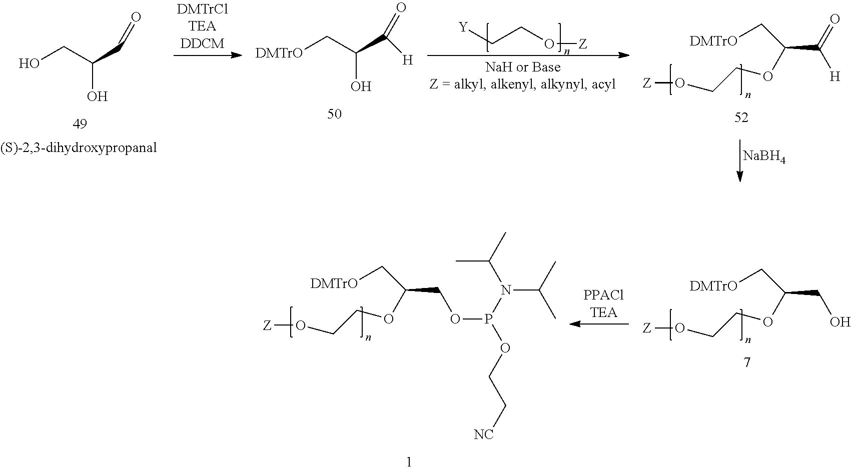

Diol 49 was dissolved in DCM and TEA. A solution of DMT-Cl in DCM was added portion-wise. MeOH was added and the reaction was dried under reduced pressure. The residue was then purified by flash chromatography to afford mono-tritylated product 50.

Secondary alcohol 50 was dissolved in anhydrous THF. Sodium hydride was added to generate alkoxide. When the bubbling ceased, mPEGn-OTs was dissolved in THF and added portion-wise. The reaction stirred vigorously at ambient temperatures overnight. Excess NaH was quenched with water, then diluted with water and extracted with EtOAc. The combined organic layers were dried under reduced pressure. The residue was resuspended in toluene, separated from remaining salts, and purified by flash chromatography to afford 52.

52 was dissolved in MeOH and cooled to 0° C. Sodium borohydride (NaBH4) was added portion-wise under an inert atmosphere and stirred for 60 minutes. Water was added and extracted with EtOAc. The combined organic layers were washed with brine, dried with sodium sulfate, filtered, and concentrated under reduced pressure. The residue was purified by flash chromatography to afford alcohol 7.

Alcohol 7 was dissolved in DCM and TEA under an inert atmosphere. PPA-Cl was added, and the reaction was stirred for 60 minutes. The reaction was dried down under reduced pressure and resuspended in toluene with 0.5% TEA, then purified by flash chromatography (Silica gel basified by 0.5% TEA mobile phase). Phosphoramidite 1 was isolated and regiopurity was established by 31P NMR.

### Example 12: Synthesis of DMT Phosphoramidites Via Dihydrobenzo Protecting Group

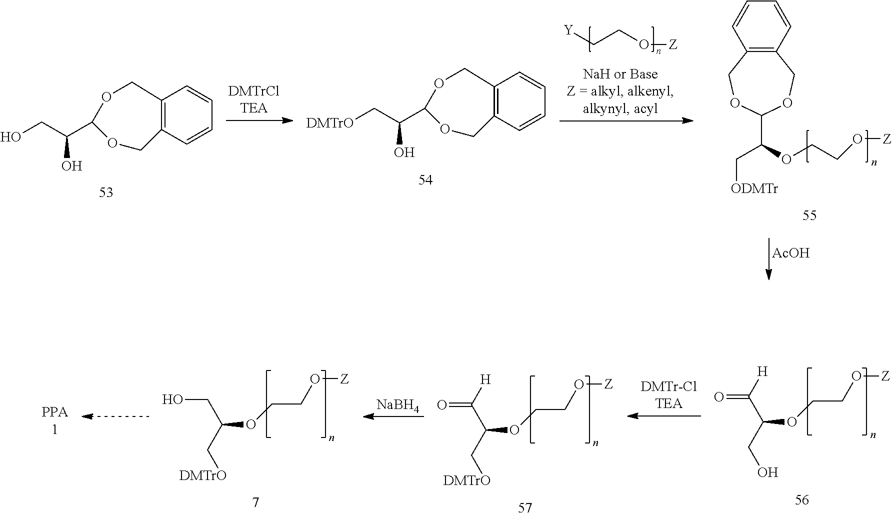

(S)-1-(1,5-dihydrobenzo[e][1,3]dioxepin-3-yl)ethane-1,2-diol 53 was dissolved in DCM and TEA. A solution of DMT-Cl in DCM was added portion-wise. MeOH was added and the reaction was dried under reduced pressure. The residue was then purified by flash chromatography to afford mono-tritylated product 54.

Secondary alcohol 54 was dissolved in anhydrous THF. Sodium hydride was added to generate alkoxide. When the bubbling ceased, mPEGn-OTs was dissolved in THF and added portion-wise. The reaction stirred vigorously at ambient temperatures overnight. Excess NaH was quenched with water, then diluted with water and extracted with EtOAc. The combined organic layers were dried under reduced pressure. The residue was resuspended in toluene, separated from remaining salts, and purified by flash chromatography to afford 55.

Product 55 was dissolved in a mixture of Acetic acid and water (2:1). The solution was heated to 40° C. and stirred for 120 minutes, then neutralized with sodium bicarbonate and extracted with EtOAc. Organics were separated, dried over Na₂SO₄. and concentrated under reduced pressure. The residue was purified by flash chromatography to afford alcohol 56.

Alcohol 56 was dissolved in DCM and TEA. A solution of DMT-Cl in DCM was added portion-wise. MeOH was added and the reaction was dried under reduced pressure. The residue was then purified by flash chromatography to afford trityl product 57.

57 was dissolved in MeOH and cooled to 0° C. Sodium borohydride (NaBH4) was added portion-wise under an inert atmosphere and stirred for 60 minutes. Water was added and extracted with EtOAc. The combined organic layers were washed with brine, dried with sodium sulfate, filtered, and concentrated under reduced pressure. The residue was purified by flash chromatography to afford the common intermediate alcohol 7.

Alcohol 7 was dissolved in DCM and TEA under an inert atmosphere. PPA-Cl was added, and the reaction was stirred for 60 minutes. The reaction was dried down under reduced pressure and resuspended in toluene with 0.5% TEA, then purified by flash chromatography (Silica gel basified by 0.5% TEA mobile phase). Phosphoramidite 1 was isolated and regiopurity was established by 31P NMR.

### Example 13: Synthesis of DMT Phosphoramidite Starting from (S)-Methyl 2,3-Dihydroxypropanoate

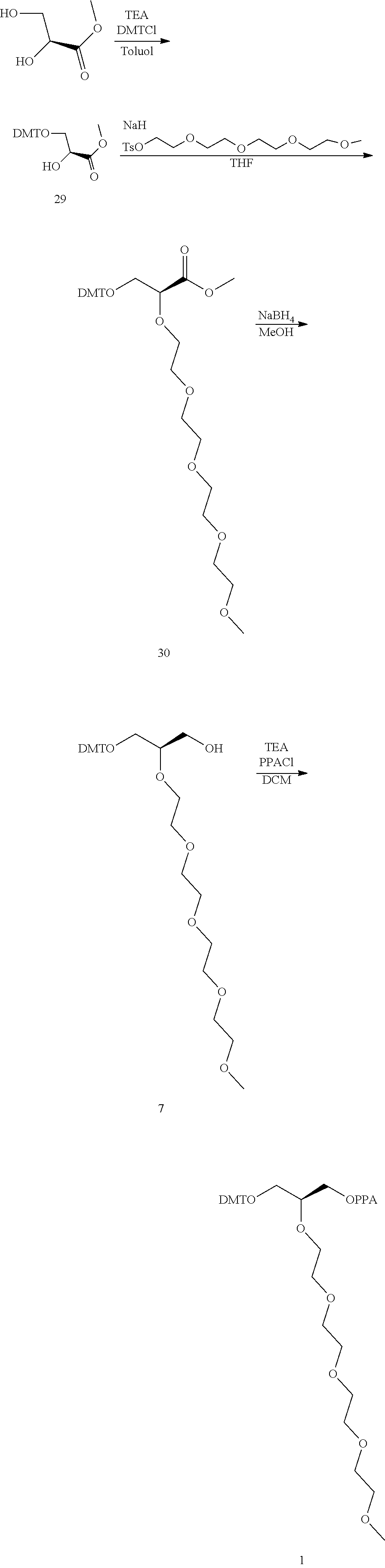

(S)-Methyl 2,3-dihydroxyglycerate was dissolved in toluene, TEA and DMTCl were added, and the reaction mixture was stirred at rt. After the reaction was completed, water was added, and the aqueous phase was extracted with toluene. The combined extracts were dried, concentrated in vacuo and the crude product was purified by flash chromatography to afford the product 29.

Hydroxy ester 29 was dissolved in THF, NaH and propargylPEG4Tos were added. After the reaction was completed, water was added, and the aqueous phase was extracted with EtOAc. The combined extracts were dried and concentrated in vacuo. The crude product was purified by flash chromatography to afford 30.

30 was dissolved in MeOH and cooled to 0° C. Sodium borohydride (NaBH4) was added portion-wise under an inert atmosphere and stirred for 60 minutes. Water was added and extracted with EtOAc. The combined extracts were dried and concentrated in vacuo. The crude product was purified by flash chromatography to afford 7.

DMT mPEG4 alcohol 7 was dissolved in DCM, TEA and PPACl were added, and the reaction mixture was stirred at rt. After the reaction was completed, water was added, and the aqueous phase was extracted with DCM. The combined extracts were dried and concentrated in vacuo. The crude product was purified by flash chromatography to afford 1.

### Example 14: Synthesis of DMT Phosphoramidite 1b Via the Azidomethyl

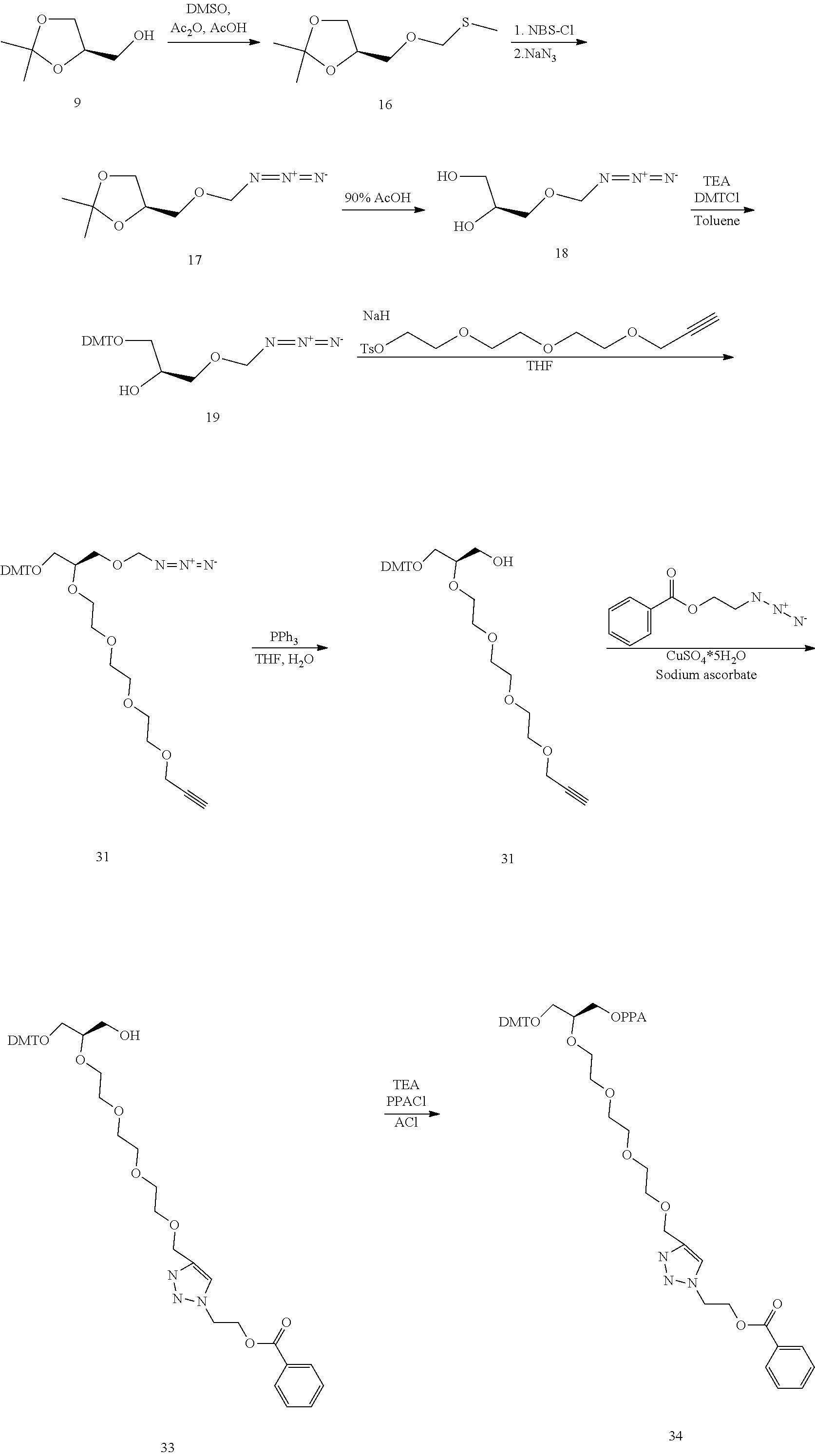

### Protecting

The azidomethyl group was introduced in the two following steps based on literature methods: (1) PNAS 2008, 105(27), 9145-9150; (2) Russian Journal of Bioorganic Chemistry 2009, 35, 270-273.

2,3-Isopropylidene-sn-glycerol 9 was dissolved in DMSO, AcOH and Ac₂O. The reaction mixture was stirred 50 h at rt. The reaction was quenched with sat. solution of NaHCO₃ and the aqueous phase was extracted with EtOAc. The EtOAc was evaporated, and the residue dried under vacuum to afford 16 as crude product in 63% yield.

The crude thio ether 16 was solved in DMF and 2-nitrobenzene sulfonyl chloride was added. After 5 min the reaction mixture was cooled to 0° C. and NaN₃ was added. After 1 h the reaction was quenched with half concentrated NaHCO₃ solution, and the aqueous phase was extracted with EtOAc. The crude product was purified by flash chromatography to afford 17 in 58% yield.

Azidomethyl ether 17 was dissolved in 90% AcOH and heated 20 min at 80° C. The solvent was removed under reduced pressure. The residue was resuspended in EtOAc and was extracted with water and brine. The solvent was evaporated to afford diol 18 in 87% yield.

Diol 18 was dissolved in toluene and TEA. A solution of DMT-Cl in toluene was added dropwise at rt. The reaction mixture was extracted with water and brine. The solvent was evaporated, and the residue then purified by flash chromatography to afford the mono-trityl product 19 in 88% yield.

Secondary alcohol 19 was dissolved in THF, NaH (60% dispersion in mineral oil) and propargylPEG4Tos was added. After the reaction was completed, water was added, and the aqueous phase was extracted with EtOAc. The combined extracts were dried and concentrated in vacuo. The crude product was purified by flash chromatography to afford 31 in 70% yield.

propargylPEG4 ether 31 was dissolved in THF, PPh₃ and H₂O were added. The reaction mixture was stirred 18 h at room temperature. The solvent was evaporated, and the crude product was purified by flash chromatography to afford 32 in 89% yield.

DMT PEG4 alcohol 32 was dissolved in EtOH and 2-azidoethyl benzoate dissolved in DMSO was added. CuSO₄ in H₂O and Sodium ascorbate in H₂O were added to the reaction mixture. After 60 min the reaction was quenched with 0.5 mol EDTA solution. The solvent was removed under reduced pressure. The residue was resuspended in H₂O and was extracted with EtOAc. The combined extracts were extracted with H₂O and brine. The solvent was evaporated, and the residue then purified by flash chromatography to afford product 33 in 93% yield.

DMT PEG alcohol 33 was dissolved in ACN and TEA. PPA-Cl was added, and the reaction was stirred 1 h at rt. The precipitate was filtered off and the solvent dried down under reduced pressure. The crude product was purified by flash chromatography to afford 34 in 64% yield. Phosphoramidite 34 was confirmed by 1H and 31P NMR. [(TLC nHex/EtOAc 25/75+0.5% TEA Rf=0.32), 1H NMR (500 MHz, ACETONITRILE-d3) δ ppm 1.05-1.15 (m, 12H) 2.18 (s, 1H) 2.56 (dt, J=12.17, 5.89 Hz, 2H) 3.07-3.17 (m, 2H) 3.48-3.56 (m, 12H) 3.60-3.76 (m, 13H) 4.53 (s, 2H) 4.63-4.68 (m, 2H) 4.67-4.73 (m, 2H) 6.82-6.87 (m, 4H) 7.17-7.22 (m, 1H) 7.26-7.34 (m, 6H) 7.42-7.48 (m, 4H) 7.58 (t, J=7.46 Hz, 1H) 7.81 (s, 1H) 7.93 (d, J=7.35 Hz, 2H), 31 P NMR (500 MHz, ACETONITRILE-d3) δ ppm 147.81, 147.87.

### Example 15—Synthesis of DMT Phosphoramidite 1a Via the Benzyl Protecting Group

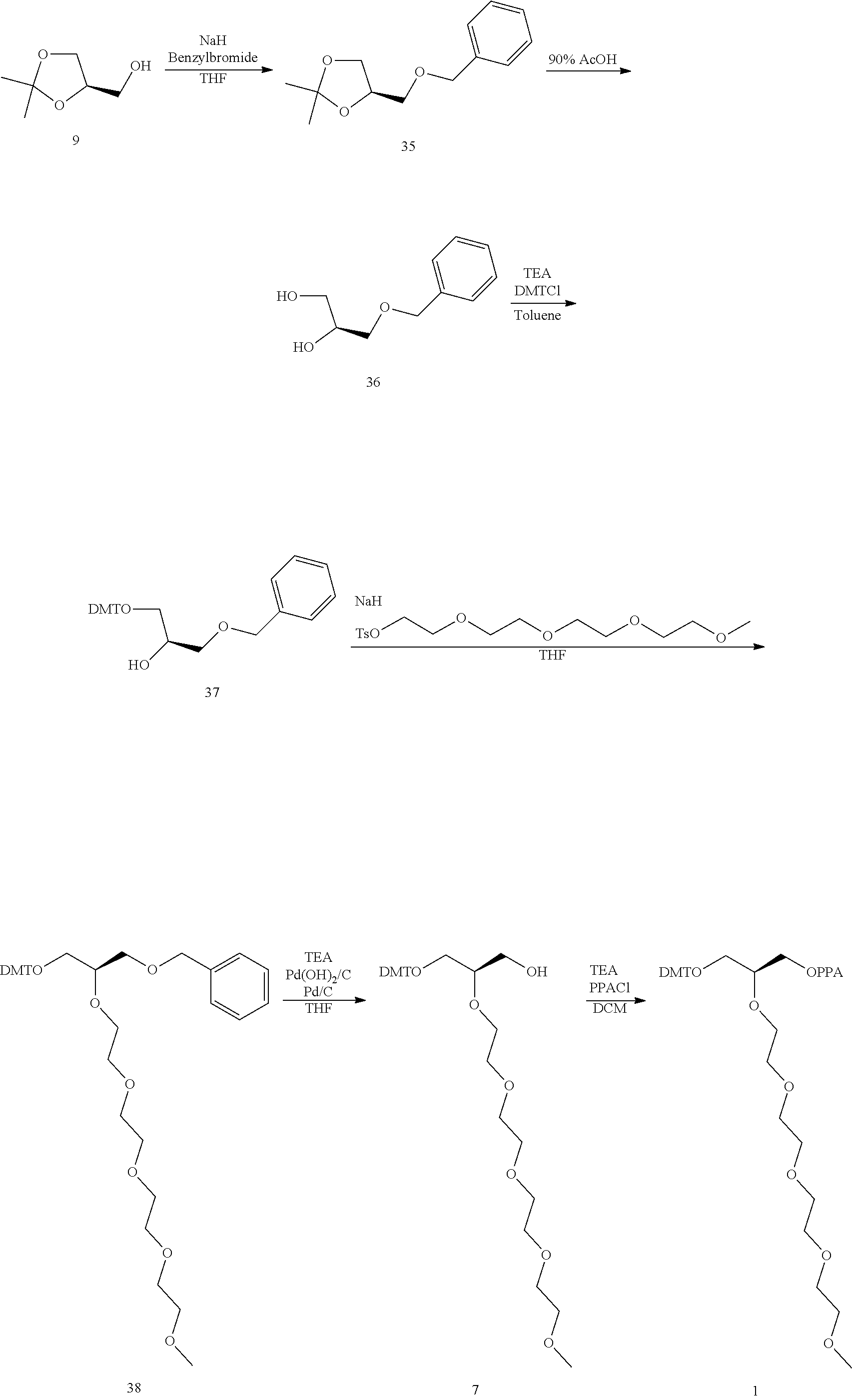

2,3-Isopropylidene-sn-glycerol 9 was dissolved in anhydrous THF, reaction mixture was cooled in an ice bath, NaH (60% dispersion in mineral oil) and BnBr were added and the reaction mixture was stirred at rt for 20 h. Water was added and the aqueous phase was extracted with EtOAc. Combined extracts were dried and concentrated in vacuo. The crude product was purified by flash chromatography to afford 35 in 89% yield.

Bn-protected ether 35 was dissolved in 90% AcOH and heated to 90° C. for 1 h and afterwards it was stirred at rt for 18 h. The reaction mixture was concentrated in vacuo and the crude product was purified by flash chromatography to afford diol 36 in 85% yield.

Diol 36 was dissolved in toluene, TEA was added, the reaction mixture was cooled in an ice bath, DMTCl was added, and the reaction mixture was stirred at rt for 18 h. Water was added, and the aqueous phase was extracted with toluene. The combined extracts were dried and concentrated in vacuo. The crude product was purified by flash chromatography to afford 37 in 78% yield.

Secondary alcohol 14 was dissolved in THF, the reaction mixture was cooled in an ice bath, NaH (60% dispersion in mineral oil) and 15 min later mPEG4Tos were added. The reaction was stirred at rt for 66 h, water was added, and the aqueous phase was extracted with EtOAc. The combined extracts were dried and concentrated in vacuo. The crude product was purified by flash chromatography to afford 38 in 91% yield.

mPEG4-Ether was dissolved in THF, 0.2 equiv TEA, 0.15 equiv Pd/C and 0.15 equiv Pd(OH)₂/C were added. The reaction mixture was stirred under the H₂-atmosphere at 70° C. for 13 h and at rt for further 18 h. The catalysts were filtered off, washed with EtOAc and the solvent was evaporated in vacuo. The crude product was purified by flash chromatography to afford 7 in 66% yield.

DMT mPEG4 alcohol 7 was dissolved in DCM, TEA and PPACl were added, and the reaction mixture was stirred at rt. After the reaction was completed, water was added, and the aqueous phase was extracted with DCM. The combined extracts were dried and concentrated in vacuo. The crude product was purified by flash chromatography to afford 1. The structure of the phosphoramidite 1 was confirmed by ¹H and ³¹P NMR (CD₃CN): 148.46 ppm, 148.42 ppm.

### Example 16—Synthesis of DMT Phosphoramidites Via the Mannitol

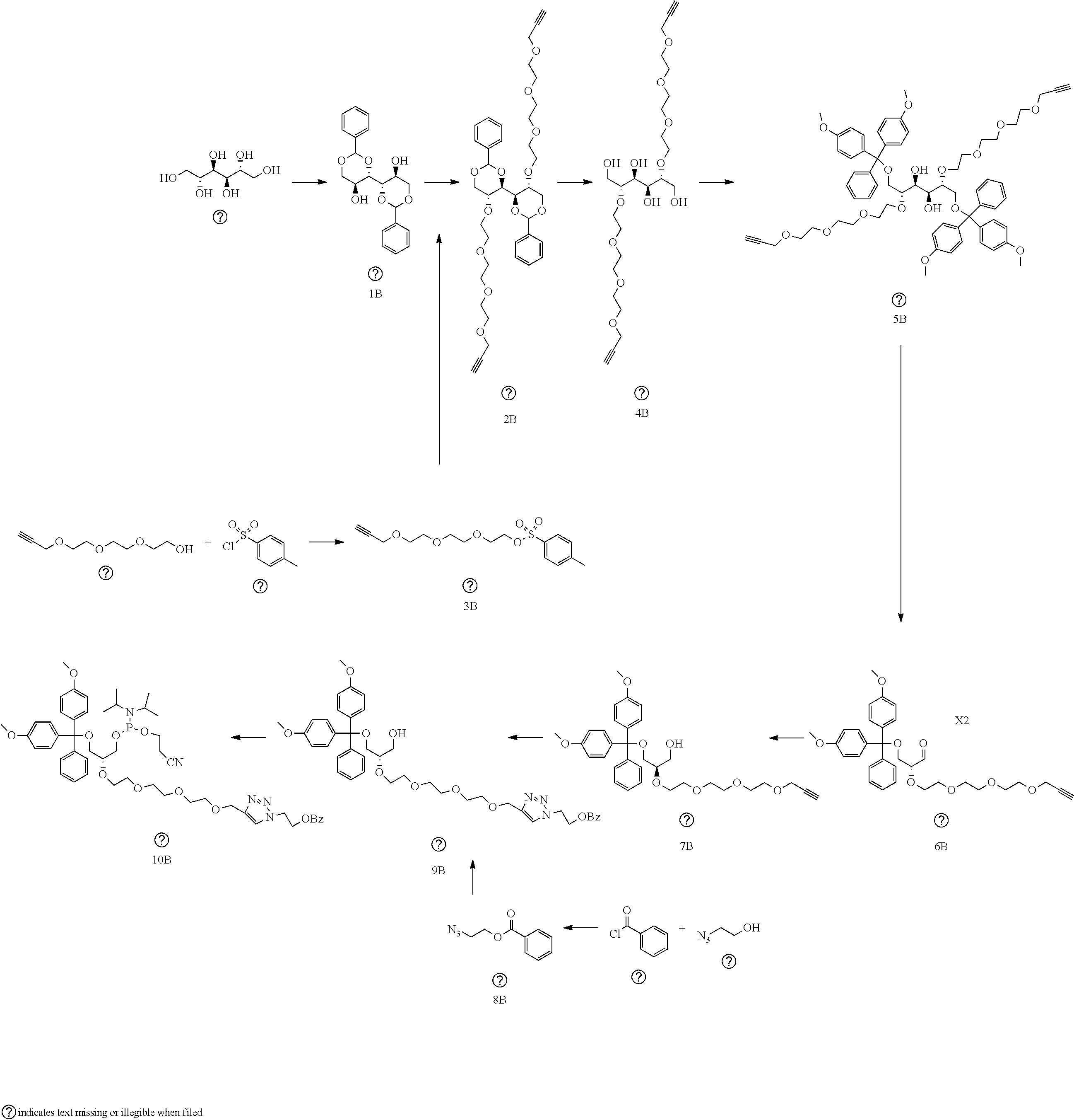

### Synthesis of Product 1B

Materials included 25 g D-Mannitol CAS: 69-65-8, 29 g Benzaldehyde CAS: 100-52-7, 10.3 mL Sulfuric Acid CAS: 7664-93-9, and 82.3 mL DMF.

To a solution of 25 g of D-Mannitol and Benzaldehyde (29.3 g, 27.9 mL) in 300 mL DMF was added 10.3 mL of concentrated Sulfuric Acid, dropwise. The initially heterogeneous mixture was stirred vigorously for 3 days at ambient temperatures and became clear. The reaction mixture was poured slowly, portion-wise, into 3 L crushed ice water containing 30 g of potassium carbonate and 500 mL hexanes. This mixture was stirred vigorously, and white precipitate formed as the ice melted. PPT was filtered off and washed with hexanes. An analytical sample was washed with cold DCM. Bulk material was dried under HIVAC overnight then recrystallized from 200 mL MeOH to afford 9 g of white powder in 18% yield.

Purification: Recrystallization from MeOH

Process Control: TLC: 5:95 MeOH in EtOAc

Yield: 9 g of Product 1B, about 18%

### Synthesis of Product 2B

Materials included 10 g 1,3:4,6-Di-O-benzylidene-D-Mannitol CAS: 28224-73-9 (Product 1B), 4.46 g Sodium Hydride (60% in mineral oil), 28.66 g Product 3, 120 mL THF.

A dried 1000 mL round bottom flask equipped with a stir bar was purged with argon. To this, 10 g of Product 1B were added followed by 120 mL of dry THF and stirred until Product 1B was fully dispersed (at this concentration the benzylidene does not seems to completely dissolve). As soon as a homogeneous dispersion was achieved, it was cooled down in an ice bath and stirred for 5 minutes. To the cold dispersion, 4.46 g of sodium hydride were added slowly letting the bubbling cease after each addition.

Next, 28.66 g of Product 3B were added slowly while stirring. After the addition was completed, the mixture was stirred in the ice bath for another 5 minutes then it was warmed up to room temperature and left under an argon atmosphere for ˜60 hrs. The reaction was monitored by TLC (3:2 EtOAc:Hex) Rf=0.38 (It should be noted that the Rf of the starting material and product were very close SM:Rf=0.44 and slight variations in the solvent system proportions could make them overlap. They could be distinguished, however; when stained using PAA, SM stains black-bluish and product stains purple-reddish).

The reaction was quenched by placing two 1 L Erlenmeyer flasks in an ice bath and then splitting the reaction mixture between them. First, 5 mL of water were added to each and quickly 100 mL of EtOAc were added too, letting the bubbling dissipate and gently swirling. Then, 5 mL more of water were added to each followed by 50 mL of EtOAc, this was repeated two to three more times until the bubbling after each water addition stopped. After, 100 mL of water was added followed by 100 mL of EtOAc, the Erlenmeyers were swirled, and let sit for 3 to 5 minutes and then more water and EtOAc was added until completing around 200 mL of water and around 400 mL of EtOAc. This mixture was transferred to a separation funnel and the organic layer was separated; the aqueous layer was then extracted 3 to 4 times with EtOAc until no more product was observed by TLC. Some brine (20-30 mL) can be added to help to separate the emulsion faster. Last, the organic layer was dried over sodium sulfate for 30 minutes.

Purification: The organic layer was decanted, and the sodium sulfate was rinsed with more EtOAc. The collected liquid was then rotavaped until obtaining a thick brown oil. This oil was left under high vacuum for 1 hour. Then loaded onto a 220 g silica cartridge using minimal DCM. (Flow: 120 mL/min; Eluent: Hex/EtOAc; Gradient Profile 6 min 100% Hex, 15 min gradient 0 to 50% EtOAc, 10 min Isocratic 50% EtOAC, 10 min gradient 50% to 65% EtOAc)

Process control: TLC: 3:2 EtOAc:Hex

NMR 1H & 13C (ACN-d3)

MS (ESI)

Yield: 16.97 g of product 2B about 87%

### Synthesis of Product 3B

Materials included 25 g Propargyl-PEG4-OH, CAS: [208827-90-11, 32.9 g Tosylchloride, 26.884 g (37.030 mL) Triethylamine, and 265.67 mL DCM.

In an argon purged round bottom flask, Propargyl-PEG4-OH was dissolved in 270 mL of DCM. Then 37 mL of TEA were added, and the solution was stirred for 5 minutes. Tosylchloride was added in portions as a solid over 10 minutes while stirring. Stirring was continued over night at ambient temperature. Process control (TLC MeOH:DCM 2%). After the reaction was complete, 10-20 mL of hexanes were added and the precipitate (triethyl ammonium chloride) was filtrated, then the solvent mixture was evaporated in a rotary evaporator until dryness and left under high vacuum for 30 minutes to an hour.

Purification: The crude oil was then loaded onto a 220 g silica cartridge using toluene (Flow: 120 mL/min; Eluent: Hex/EtOAc; Gradient Profile 5 min Isocratic 20% EtOAc, 15 min gradient 20 to 60% EtOAc, 5 min Isocratic 60% EtOAc, 5 min gradient 60% to 100% EtOAc, 5 min Isocratic 100% EtOAc)

Yield: about 77%, 34.9 g of product 3B

### Synthesis of Product 4B

Materials included 16.975 g Benzylidene-D-Mannitol-di-4PEGAlkyne, 28 g (23.3 mL) Hydrochloric Acid 37%, 173.5 mL Methanol, and 28.7 g (39.6 mL) of Triethylamine.

175 mL of MeOH were added to a round bottom flask purged with argon containing Product 2B. After dissolving product 2B, 23.3 mL of HCl 37% were added and stirred keeping the flask under an argon static atmosphere. The reaction was left for 15 hours at RT. Process control (TLC EtOAc:Hex 6:1) Rf: 0.15. In an attempt to push the equilibrium towards the products and maximize yield, 100 mL of diethylether, 150 mL of water and 50 mL of hexanes were added forming a hydrophobic layer on top to push some benzaldehyde into this layer. The reaction was left under these conditions for 2 more hours (top layer became slightly yellowish). Work-up: First, the reaction was neutralized, titrating with triethylamine until reaching an observable pH of ˜6.5-7.5 (pH strip, bottom layer).

After neutralization, the more volatile components were evaporated in a rotary evaporator collecting 150 mL-200 mL of liquid including MeOH. The remaining layer was transferred to a separation funnel and 200 mL of water were added followed by 50 mL of diethyl ether (to extract benzaldehyde and un-polar residues). This mixture was shaken, and the organic layer becomes yellow, then it was separated.

Later, 50 mL more of diethyl ether were added and the process was repeated. The aqueous layer was kept apart and then the collected organic layers were back extracted by adding 10 mL of hexanes (organic layer becomes cloudier) and extracting twice with 30 mL of water. TLC showed that both aqueous layers (original and back extracted one) contains mainly the product and the organic one contains mainly benzaldehyde and some starting material. The aqueous layers were combined, and the water was evaporated in a rotary evaporator resulting in the formation of a precipitated (triethyl ammonium chloride) along with an oil (Product 4B). This oil-salt mixture was azeotroped with acetonitrile and dried under high vacuum and kept under an argon atmosphere.

The resulting dried oil-salt mixture was taken as it was to the next step without further treatment.

Process control: TLC: EtOAc:Hex 6:1

NMR 1H & 13C (ACN-d3)

MS (ESI)

Yield: Assumed about 100% for the next reaction.

### Synthesis of Product 5B

Materials included 12.7 g D-Mannitol-di-4PEGAlkyne (Product 4B), 16.05 g DMT-Cl, 7.37 g (10.16 mL) Triethylamine, and 40 mL DCM.

The flask containing the dried product 4B and triethyl ammonium chloride was purged with fresh argon and equipped with a stir bar. Then, 40 mL of DCM were added to dissolve product 4B, after 10.2 mL of triethylamine were added and stirred for 2 minutes. To this 16.05 g of DMT-Cl were added as a solid in portions over 5 minutes. The reaction turns reddish then yellow, and later greenish. The reaction was monitored by TLC (4:1 EtOAc:Hex Rf: 0.55 or 5% MeOH:DCM Rf: 0.41) until completion.

Work-up: 10 mL of Hexanes were added and the triethylammonium chloride were filtered through a frit, collected crystals were rinsed with 20 mL of DCM then the solvent was evaporated in a rotary evaporator until dryness.

Purification (The crude oil was then loaded onto a 220 g silica cartridge using toluene filtering the remaining triethylammonium chloride; Flow: 120 mL/min; Eluent: Hex/EtOAc; Gradient Profile: 8 min Isocratic 100% Hex, 15 min gradient 0 to 50% EtOAc, 3 min Isocratic 50% EtOAc, 10 min gradient 50% to 85% EtOAc, 6 min Isocratic 100% EtOAc)

Process control: NMR 1H & 13C (CAN-d3), MS (ESI)

Yield: 59% 16 g of product 5B (two steps)

### Synthesis of Product 7B

Materials included 16 g D-Mannitol-di-4PEGAlkyne-Di-DMT (Product 5B), 4.55 g NaIO₄, 2.02 g (2.07 mL) Pyridine, 85 mL THF, and 15 mL water.

The round bottom flask containing product 5B was purged with fresh argon and product 5B was dissolved in 85 mL of THF. After complete dissolution, 2.1 mL of pyridine were added and stirred for 1 minute then 4.55 g of NaIO₄ were added while stirring. Then, water was added in 3 mL portions and a white precipitate started to form. Since DMT could come off in the presence of NaIO₄/NaIO₃/H₂O, it was imperative monitoring the progress of the reaction and adjusting the amount of water and pyridine accordingly if major DMT byproduct was observed by TLC.

At this scale under these conditions the reaction took 6 to 7 hours to complete but more NaIO₄ could be added or even slightly heated it (about 40° C.) to speed up the reaction. Of course, it was done, the water and pyridine ratios would also have to be adjusted to avoid degradation. TLC (5:1 EtOAc:Hex Rf: 0.68). After the reaction appeared completed, the white solid was filtered and more THF (˜180 mL) was added and the resulting solution was taken without further treatment to the next step, assuming 200% yield (2 eq formed).

### Reduction

16 g (Assumed) DMT-4PEGAlkyne-Aldehyde (Product 6B)

1.07 g NaBH4

283 mL THF

The filtrate from the oxidation was collected in an argon-purged round bottom flask equipped with a stir bar. This solution was cooled down to 0° C. in an ice bath, then 1.07 g of NaBH4 were added while keeping the vessel in the ice bath for the first part of the reaction, after 15 minutes the flask was removed from the ice bath and left at room temperature while monitoring by TLC (5:1 EtOAc:Hex Rf: 0.48). Once the reaction was complete it was quenched with acetone and 100 mL of water was added and product 7B was extracted with EtOAc 4 times with 50 mL (10 mL brine can be added to the aqueous layer to improve results). Then, the isolated organic extractions were dried over sodium sulfate and later evaporated in a rotary evaporator.

### Purification

The crude oil was then loaded onto a 220 g silica cartridge using toluene.

Flow: 120 mL/min

Eluent: Hex/EtOAc then EtOAc/MeOH*

### Gradient Profile

10 min Isocratic 100% Hex

28 min gradient 0 to 100% EtOAc

3 min Isocratic 100% EtOAc

10 min gradient 0% to 5% MeOH*

10 min Isocratic 5% MeOH

Process control: NMR 1H & 13C (ACN-d3), MS (ESI)

Yield 166% 13.3 g of Product 7B over two steps (2 eq produced)

### Synthesis of Product 8B

Materials included 20 g 2-Azidoethanol, 41.9 g Benzoyl chloride, 75.5 g (104.1 mL) Triethylamine, and 306.27 mL DCM.

20 g of 2-Azidoethanol were diluted with DCM in a round bottom flask equipped with a stir bar. Under an argon blanket 75.5 g (104.1 mL) of TEA were added followed by adding benzoyl chloride slowly. The reaction proceeded for 16-24 hours at room temperature monitoring by TLC (1:4 EtOAc:Hex Rf: 0.61). Work-up: 30 mL of hexanes were added then the precipitate was filtered (triethylammonium chloride) using a frit, rinsed with DCM, and evaporated in rotary evaporator.

### Purification

The crude oil was then loaded neat onto a 220 g silica cartridge, first filtering the remaining triethylammonium chloride and chasing with hexanes.

Flow: 120 mL/min

Eluent: Hex/EtOAc

Gradient Profile

5 min Isocratic 100% Hex

5 min gradient 0 to 5% EtOAc

15 min Isocratic 5% EtOAc

5 min gradient 5% to 20% EtOAc

10 min Isocratic 20% EtOAc

Process control: NMR 1H & 13C (ACN-d3)

Yield: Quantitative

### Synthesis of Product 9B

Materials included 13.4 g DMT-Gly-4PEGalkyne-OH (Product 7B), 5.87 g Azido-ethyl-benzoyl (Product 8B), 9.36 g Sodium Ascorbate, 1.18 g CuSO4 Pentahydrate, 53 mL water, and 107.4 mL DMSO.

In separate flasks, the catalyst mix was prepared by dissolving sodium ascorbate in water/DMSO and dissolving CuSO4 Pentahydrate in water. The remaining DMSO (2/3) was used to dissolve 13.4 g of Product 7B under an argon blanket in a round bottom flask. The sodium ascorbate and CuSO4 solutions were combined first; meanwhile 5.87 g of Azido-ethyl-benzoyl (Product 8B) was added to the round bottom flask. After stirring for 3 minutes, the catalyst mix was added to the solution of product 7B and 8B. The reaction was left at room temperature and monitored by TLC (100% EtOAc Rf: 0.25 or 5% MeOH:DCM Rf: 0.38).

Quenching: The reaction was stopped using a solution of EDTA 0.5 Molar; pH 8, then EtOAc 200 mL, water 200 mL and brine 50 mL were added, and the mixture was swirled until the yellow/brownish solids were dissolved and a blue/greenish layer (aq) and a clear layer (org) was obtained. This was transferred to a separation funnel and the aqueous layer was washed 4 times with EtOAc. The combined organic fractions were dried over sodium sulfate then decanted and the liquid was evaporated in a rotary evaporator.

### Purification

The crude oil was then loaded neat onto a 220 g silica cartridge using toluene

Flow: 120 mL/min

Eluent: Hex/EtOAc then EtOAc/MeOH*

### Gradient Profile

5 min Isocratic 100% Hex

20 min gradient 0 to 100% EtOAc

10 min Isocratic 100% EtOAc

8 min gradient 0% to 6% MeOH*

12 min gradient 6% to 10% MeOH

Process control: NMR 1H, TLC

Yield 75% 13.4 g of Product 9B

### Synthesis of Product 10B

Materials included 13.4 g Product 9B, 3.76 g (3.54 mL) 2-Cyanoethyl N,N-diisopropylchlorophosphoramidite (PPA-Cl), 3.57 (4.92 mL) Triethylamine, 100.8 mL DCM, and 60 mL Toluene.

Product 9B was dried extensively under high vacuum (1 day). The day of the reaction the flask containing product 9B was filled with argon and product 9B was dissolved in dry DCM. Then PPA-Cl 3.76 g (3.54 mL) and triethylamine (4.92 mL) were measured in a glovebox. First triethylamine was added to the flask under positive argon pressure and stirred for 1 minute, then the PPA-Cl was added slowly also under positive argon pressure. The reaction was left stirring at room temperature under argon and monitored by TLC (1% Triethylamine in EtOAc Rf: 0.67). When the reaction was completed, the solvent was evaporated in a rotary evaporator backfilling the round bottom flask with argon as vacuum was released, the product was then kept in high vacuum before purification.

### Purification

Dry toluene was transferred to 50 mL falcon tubes after evacuating most of the air and creating an argon blanket, then drying traps were submerged in the solvent for further/maintaining dryness. Product 10B was dissolved in dry toluene and loaded onto a 220 g silica cartridge.

Flow: 120 mL/min

Eluent: Hex 0.5% TEA/EtOAc 0.5% TEA

Drying traps were added to each solvent at least 1 hour before the purification and dry TEA was used for basification of hexanes and EtOAc.

Extended equilibration (14 minutes) to ensure silica-cartridge neutralization before the run.

### Gradient Profile

8 min Isocratic 100% (Hex 0.5% TEA)

12 min gradient 0 to 66% (EtOAc 0.5% TEA)

20 min Isocratic 100% (EtOAc 0.5% TEA)

Process control: NMR 1H & 31P (ACN-d3)

Yield: 71% of product 10B

### Example 17—Synthesis of DMT Phosphoramidites Via the Mannitol

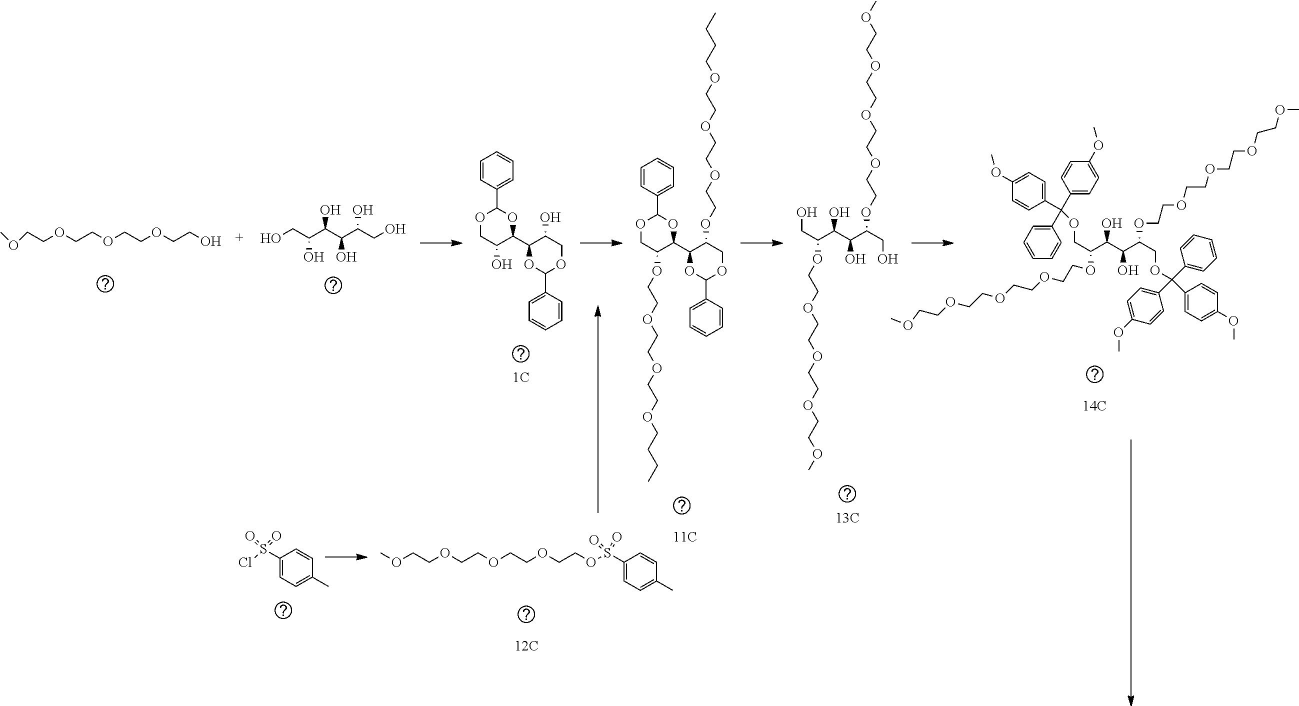

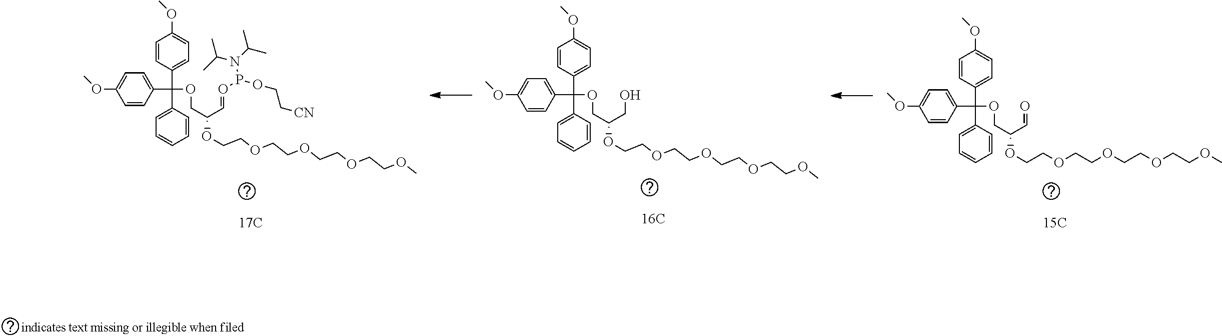

### Synthesis of Product 11C

Materials included 10 g 1,3:4,6-Di-O-benzylidene-D-Mannitol CAS: 28224-73-9 (Product 1C) (see procedure in Example 16 for the synthesis of product 1B), 4.46 g Sodium Hydride (60% in mineral oil), 30.34 g Product 12C (see procedure below), and 120 mL THF.

A dried 1000 mL round bottom flask equipped with a stir bar was purged with argon. To this, 10 g of Product 1C were added followed by 120 mL of dry THF and stirred until Product 1C was fully dispersed (at this concentration the benzylidene does not seems to completely dissolve). As soon as a homogeneous dispersion was achieved, it was cooled down in an ice bath and stirred for 5 minutes. To the cold dispersion, 4.46 g of sodium hydride were added slowly letting the bubbling cease after each addition. Then, 28.66 g of Product 12C were added slowly while stirring. After the addition was completed, the mixture was stirred in the ice bath for another 5 minutes then it was warmed up to room temperature and left under an argon atmosphere for about 60 hrs. The reaction was monitored by TLC (3:2 EtOAc:Hex) Rf=0.12.

The reaction was quenched by placing two 1 L Erlenmeyers in an ice bath and then splitting the reaction mixture between them. First, 5 mL of water were added to each and quickly 100 mL of EtOAc were added too, letting the bubbling dissipate and gently swirling. Then, 5 mL more of water were added to each followed by 50 mL of EtOAc, this was repeated two to three more times until the bubbling after each water addition stopped. After, 100 mL of water was added followed by 100 mL of EtOAc, the Erlenmeyers were swirled, and let sit for 3 to 5 minutes and then more water and EtOAc was added until completing around 200 mL of water and around 400 mL of EtOAc. This was transferred to a separation funnel and the organic layer was separated; the aqueous layer was then extracted 3 to 4 times with EtOAc until no more product was observed by TLC. Some brine (20-30 mL) can be added to help to separate the emulsion faster. Last, the organic layer was dried over sodium sulfate for 30 minutes.

### Purification

The organic layer was decanted, and the sodium sulfate was rinsed with more EtOAc. The collected liquid was then rotavaped until obtaining a thick yellow oil. This oil was left under high vacuum for 1 hour. Then loaded onto a 220 g silica cartridge using minimal DCM.

Flow: 120 mL/min

Eluent: Hex/EtOAc

### Gradient Profile

6 min 100% Hex

15 min gradient 0 to 55% EtOAc

10 min Isocratic 55% EtOAC

15 min gradient 55% to 100% EtOAc

35 min Isocratic 100% EtOAc

### Process Control:

TLC: 3:2 EtOAc:Hex

NMR 1H & 13C (ACN-d3)

MS (ESI)

Yield: 79% 16.18 g of product 11C

### Synthesis of Product 12C

Materials included 25 g m-PEG4-OH, CAS: [23783-42-8], 29.75 g Tosylchloride, 24.29 g (33.4 mL) Triethylamine, and 240.1 mL DCM.

In an argon purged round bottom flask m-PEG4-OH was dissolved in 240 mL of DCM. Then 33 mL of TEA were added, and the solution was stirred for 5 minutes. Tosylchloride was added in portions as a solid over 10 minutes while stirring. Stirring was continued over night at ambient temperature. Process control (TLC MeOH:DCM 2%). After the reaction was complete, 10-20 mL of hexanes were added and the precipitate (triethyl ammonium chloride) was filtrated, then the solvent mixture was evaporated in a rotary evaporator until dryness and left under high vacuum for 30 minutes to an hour.

### Purification

The crude oil was then loaded onto a 220 g silica cartridge using toluene.

Flow: 120 mL/min

Eluent: Hex/EtOAc

Gradient Profile

3 min Isocratic 100% Hex

5 min gradient 20% EtOAc

5 min Isocratic 20% EtOAc

16 min gradient 20 to 60% EtOAc

5 min Isocratic 60% EtOAc

5 min gradient 60% to 100% EtOAc

5 min Isocratic 100% EtOAc

Yield: 75% 32.6 g of product 12C

### Synthesis of Product 13C

Materials included 16.18 g Benzylidene-D-Mannitol-di-4PEGAlkyne, 25.25 g (21.04 mL) Hydrochloric Acid 37%, 156.4 mL Methanol, and 25.9 g (35.7 mL) of Triethylamine.

156 mL of MeOH were added to a round bottom flask purged with argon containing Product 11C. After dissolving product 11C, 21 mL of HCl 37% were added and stirred keeping the flask under an argon static atmosphere. The reaction was left for 15 hours at RT. Process control (TLC EtOAc:Hex 6:1) Rf: 0.05. In an attempt to push the equilibrium towards the products and maximize yield, 100 mL of diethylether, 150 mL of water and 50 mL of hexanes were added forming a hydrophobic layer on top to push some benzaldehyde into this layer. The reaction was left under these conditions for 2 more hours (top layer became slightly yellowish). Work-up: First, the reaction was neutralized, titrating with triethylamine until reaching an observable pH of ˜6.5-7.5 (pH strip, bottom layer).

After neutralization, the more volatile components were evaporated in a rotary evaporator collecting 150 mL-200 mL of liquid including MeOH. The remaining layer was transferred to a separation funnel and 200 mL of water were added followed by 50 mL of diethyl ether (to extract benzaldehyde and un-polar residues). This mixture was shaken, and the organic layer becomes yellow, then it was separated. Later, 50 mL more of diethyl ether were added and the process was repeated. The aqueous layer was kept apart and then the collected organic layers were back extracted by adding 10 mL of hexanes (organic layer becomes cloudier) and extracting twice with 30 mL of water. TLC showed that both aqueous layers (original and back extracted one) contains mainly the product and the organic one contains mainly benzaldehyde and some starting material. The aqueous layers were combined, and the water was evaporated in a rotary evaporator resulting in the formation of a precipitated (triethyl ammonium chloride) along with an oil (Product 13C). This oil-salt mixture was azeotroped with acetonitrile and dried under high vacuum and kept under an argon atmosphere.

The resulting dried oil-salt mixture was taken as it was to the next step without further treatment.

### Process Control:

TLC: EtOAc:Hex 6:1

NMR 1H & 13C (CAN-d3)

MS (ESI)

Yield: Assumed 100% for the next reaction.

### Synthesis of Product 14C

Materials included 12.3 g D-Mannitol-di-mPEG4 (Product 13C), 14.47 g DMT-Cl

6.65 g (9.16 mL) Triethylamine, and 36 mL DCM.

The flask containing the dried product 13C and triethyl ammonium chloride was purged with fresh argon and equipped with a stir bar. Then, 36 mL of DCM were added to dissolve product 13C, after 9.2 mL of triethylamine were added and stirred for 2 minutes 14.47 g of DMT-Cl were added as a solid, in portions over 5 minutes. The reaction turns reddish then yellow, and later greenish. The reaction was monitored by TLC (4:1 EtOAc:Hex Rf: 0.14 or 5% MeOH:DCM Rf: 0.3) until completion.

Work-up: 10 mL of Hexanes were added and the triethylammonium chloride were filtered through a frit, collected crystals were rinsed with 20 mL of DCM then the solvent was evaporated in a rotary evaporator until dryness.

### Purification

The crude oil was then loaded onto a 220 g silica cartridge using toluene filtering the remaining triethylammonium chloride.

Flow: 120 mL/min

Eluent: Hex/EtOAc then EtOAc/MeOH*

### Gradient Profile

8 min Isocratic 100% Hex

15 min gradient 0 to 75% EtOAc

10 min Isocratic 75% EtOAc

5 min gradient 75% to 100% EtOAc

1 min Isocratic 100% EtOAc

1 min gradient 0% to 5% MeOH*

20 min Isocratic 5% MeOH

Process control: NMR 1H & 13C (ACN-d3), MS (ESI)

Yield: 72% 18.5 g of product 14C

### Synthesis of Product 16C

### Oxidative Cleavage

Materials included 18.505 g D-Mannitol-di-m4PEG-Di-DMT (Product 14C), 5.09 g NaIO₄, 2.26 g (2.31 mL) Pyridine, 95 mL THF, and 17 mL water.

The round bottom flask containing product 14C was purged with fresh argon and product 14C was dissolved in 95 mL of THF. After complete dissolution, 2.3 mL of pyridine were added and stirred for 1 minute then 5.09 g of NaIO₄ were added while stirring. Then, water was added in 3 mL portions and a white precipitate started to form. Since DMT can come off in the presence of NaIO₄/NaIO₃/H₂O, it was imperative to monitor the progress of the reaction and to adjust the amount of water and pyridine accordingly if major DMT byproduct was observed by TLC.

At this scale under these conditions the reaction takes 6 to 7 hours to complete but more NaIO₄ could have been added or even slightly heated it (˜40° C.) to speed up the reaction. If this was done water and pyridine ratios would also have needed to be adjusted to avoid degradation. TLC (5:1 EtOAc:Hex Rf: 0.22). After the reaction appeared completed, the white solid was filtered and more THF (˜150 mL) was added and the resulting solution was taken without further treatment to the next step, assuming 200% yield (2 eq formed).

As an alternative to NaIO₄, any reagent that may oxidize vicinal diols in a similar manner may be utilized. Suitable reagents include, but are not limited to, IBX (2-Iodoxybenzoic acid), Dess-Martin Periodinane, Pb(OAc)₄ or periodic acid and its several salt forms, etc.

### Reduction

### Materials Included

18.5 g (Assumed) DMT-m4PEG-Aldehyde (Product 15C), 1.24 g NaBH4, and 328 mL THF.

The filtrate from the oxidation was collected in an argon purged round bottom flask equipped with a stir bar. This solution was cooled down to 0° C. in an ice bath, then 1.24 g of NaBH4 were added while keeping the vessel in the ice bath for the first part of the reaction, after 15 minutes the flask was removed from the ice bath and left at room temperature while monitoring by TLC (5:1 EtOAc:Hex Rf: 0.11). Once the reaction was completed it was quenched with acetone, 100 mL of water are added and product 16C aw extracted with 50 mL of EtOAc 4 times (10 mL brine can be added to the aqueous layer to improve results). Then, the isolated organic extractions were dried over sodium sulfate and later evaporated in a rotary evaporator.

### Purification

The crude oil was then loaded onto 220 g silica cartridge using toluene.

Flow: 120 mL/min

Eluent: Hex/EtOAc then EtOAc/MeOH*

### Gradient Profile

7 min Isocratic 100% Hex

30 min gradient 0 to 100% EtOAc

10 min Isocratic 100% EtOAc

10 min gradient 0% to 5% MeOH*

15 min gradient 5% to 8% MeOH

Process control: NMR 1H & 13C (ACN-d3), MS (ESI)

### Synthesis of Product 17C

Materials included 14.9 g Product 16C, 5.42 g (5.11 mL) 2-Cyanoethyl N,N-diisopropylchlorophosphoramidite (PPA-Cl), 5.15 (7.09 mL) Triethylamine, 145 mL DCM, and 70 mL Toluene.

Product 16C was dried extensively under high vacuum (1 day). The day of the reaction the flask containing product 16C was filled with argon and product 16C was dissolved in dry DCM. Then PPA-Cl 5.42 g (5.11 mL) and triethylamine (7.09 mL) were measured in a glovebox. First, triethylamine was added to the flask under positive argon pressure and stirred for 1 minute, then the PPA-Cl was added slowly also under positive argon pressure. The reaction was left stirring at room temperature under argon and monitored by TLC (1% Triethylamine in EtOAc Rf: 0.7). When the reaction was completed, the solvent was evaporated in a rotary evaporator backfilling the round bottom flask with argon as vacuum was released, the product was then kept in high vacuum before purification.

### Purification

Dry toluene was transferred to 50 mL falcon tubes, after evacuating most of the air and creating an argon blanket, then drying traps were submerged in the solvent for further/maintaining dryness. Product 17C was dissolved in dry toluene and loaded onto a 220 g silica cartridge.

Flow: 120 mL/min

Eluent: Hex 0.5% TEA/EtOAc 0.5% TEA

Drying traps were added to each solvent 1 hour before the purification and dry TEA was used for basification of hexanes and EtOAc.

Extended equilibration (14 minutes) to ensure silica-cartridge neutralization before the run.

### Gradient Profile

5 min Isocratic 100% (Hex 0.5% TEA)

10 min gradient 0 to 50% (EtOAc 0.5% TEA)

15 min Isocratic 50% (EtOAc 0.5% TEA)

Process control: NMR 1H & 31P (ACN-d3)

Yield: 73% 14.6 g of product 17C

### Example 18: Synthesis of DMT Phosphoramidite Via the MTM Protecting

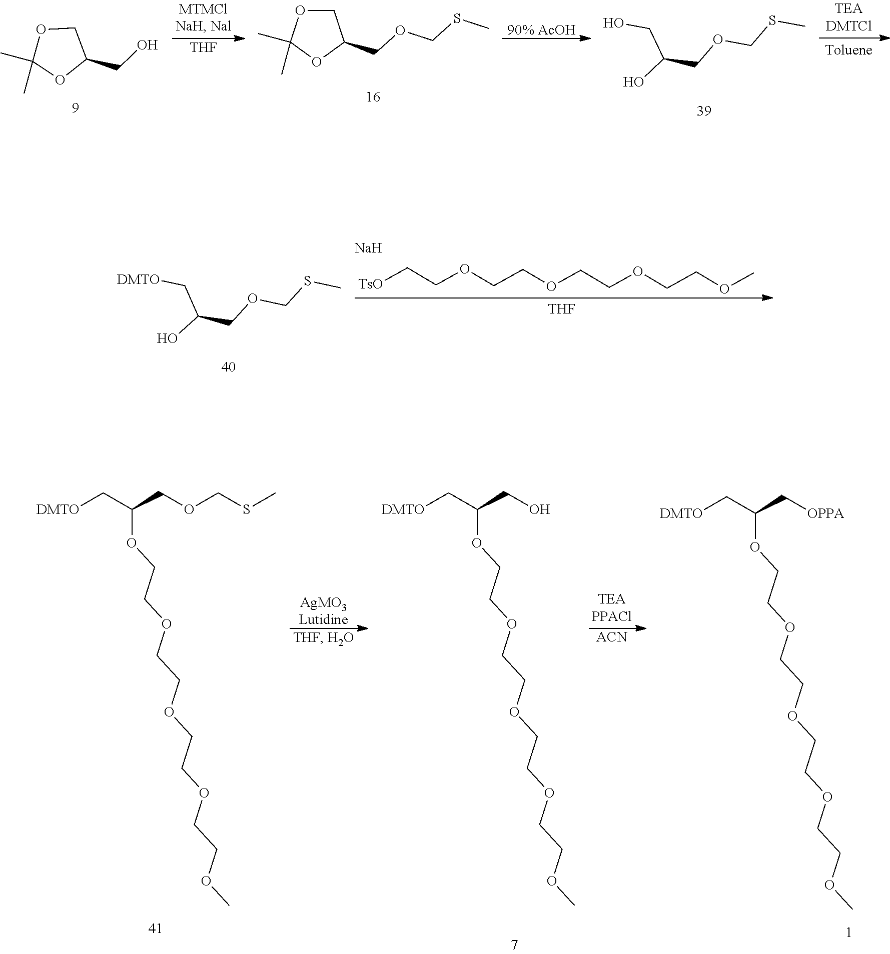

2,3-Isopropylidene-sn-glycerol 9 was dissolved in anhydrous THF and cooled to 0° C., NaH (60% dispersion in mineral oil), MTM-C₁ and NaI were added and stirred at rt. After the reaction was completed, 5% aqueous NH4Cl was added, and the aqueous phase was extracted with EtOAc. The combined extracts were dried and concentrated in vacuo. The crude product was purified by flash chromatography to afford 16 in 76% yield.

The thio ether 16 was dissolved in 90% AcOH and heated 20 min at 80° C. The solvent was removed under reduced pressure. The residue was resuspended in EtOAc and was extracted with water and brine. The solvent was evaporated to afford diol 39 in 90% yield.

Diol 39 was dissolved in toluene and TEA. A solution of DMT-Cl in toluene was added dropwise at rt. The reaction mixture was extracted with water and brine. The solvent was evaporated, and the residue then purified by flash chromatography to afford the mono-trityl product 40 in 60% yield.

Secondary alcohol 40 was dissolved in THF, NaH (60% dispersion in mineral oil) and mPEG4Tos was added. After the reaction was completed, water was added, and the aqueous phase was extracted with EtOAc. The combined extracts were dried and concentrated in vacuo. The crude product was purified by flash chromatography to afford 41 in 63% yield.

mPEG4 ether 41 was dissolved in THF/H₂O 4/1, lutidine and AgNO₃ in 100 times excess were added. After 3 h, the precipitate was filtered off. The solvent was evaporated, and the crude product was purified by flash chromatography to afford alcohol 7 in 60% yield.

Alcohol 7 was dissolved in ACN and TEA. PPA-Cl was added, and the reaction was stirred for 1 hour at rt. The precipitate was filtered off and the solvent dried down under reduced pressure. The crude product was purified by flash chromatography to afford 1 in 60% yield. The structure of the phosphoramidite 1 was confirmed by 1H and 31P NMR (CD3CN). No regioisomers were detected.

### Example 19: Synthesis of DMT Phosphoramidite 1b Via the MTM Protecting

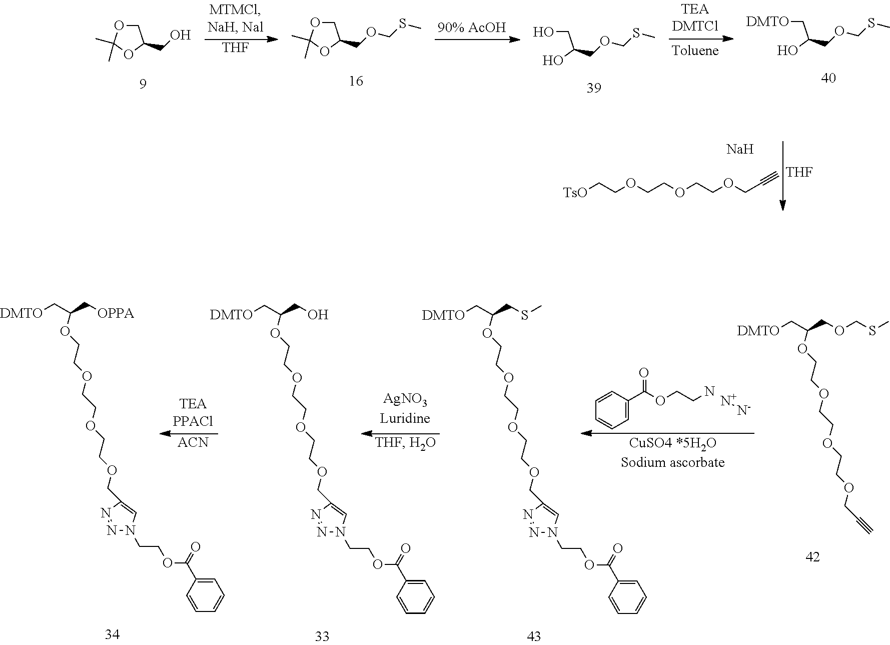

2,3-Isopropylidene-sn-glycerol 9 was dissolved in anhydrous THF and cooled to 0° C., NaH (60% dispersion in mineral oil), MTMCl and NaI were added and stirred at rt. After the reaction was completed, 5% aqueous NH₄Cl was added, and the aqueous phase was extracted with EtOAc. The combined extracts were dried and concentrated in vacuo. The crude product was purified by flash chromatography to afford 16 in 76% yield.

The thio ether 16 was dissolved in 90% AcOH and heated 20 min at 80° C. The solvent was removed under reduced pressure. The residue was resuspended in EtOAc and was extracted with water and brine. The solvent was evaporated to afford diol 39 in 90% yield.

Diol 39 was dissolved in toluene and TEA. A solution of DMT-Cl in toluene was added dropwise at rt. The reaction mixture was extracted with water and brine. The solvent was evaporated, and the residue then purified by flash chromatography to afford the mono-trityl product 40 in 60% yield.

Secondary alcohol 40 was dissolved in THF, NaH (60% dispersion in mineral oil) and pPEG4Tos was added. After the reaction was completed, water was added, and the aqueous phase was extracted with EtOAc. The combined extracts were dried and concentrated in vacuo. The crude product was purified by flash chromatography to afford 42 in 72% yield.

DMT PEG4 MTM protected alcohol 42 was dissolved in EtOH and 2-azidoethyl benzoate dissolved in DMSO was added. CuSO4 in H2O and Sodium ascorbate in H2O were added to the reaction mixture. After 60 min the reaction was quenched with 0.5 mol EDTA solution. The solvent was removed under reduced pressure. The residue was resuspended in H2O and was extracted with EtOAc. The combined extracts were extracted with H2O and brine. The solvent was evaporated, and the residue then purified by flash chromatography to afford product 43 in 68% yield.

Compound 43 was dissolved in THF/H₂O 4/1, Lutidine and AgNO₃ in 20 times excess were added. After 4 h at 40° C. the precipitation was filtered off. The solvent was evaporated, and the crude product was purified by flash chromatography to afford 33 in 54% yield.

DMT PEG alcohol 33 was dissolved in ACN and TEA. PPA-Cl was added, and the reaction was stirred 1 h at rt. The precipitate was filtered off and the solvent dried down under reduced pressure. The crude product was purified by flash chromatography to afford 34 in 76% yield. The structure of the phosphoramidite 34 was confirmed by 1H and 31P NMR (CD3CN): 148.42 ppm, 148.37 ppm.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

1. A compound having the structure of any one of Formulas (IA) and (IB): 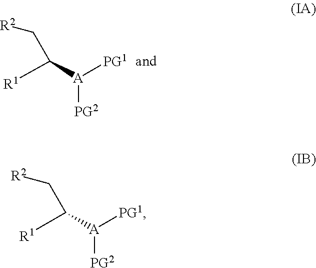
wherein
R¹ is —OH, —O—R^{w}—Z, where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, or —CH₂—Het, where “Het” is a substituted or unsubstituted 5-membered heterocyclic moiety;
R² is —OH, —O-trityl or a derivative or analog thereof, —O-9-phenylthioxanthyl (pixyl) or a derivative or analog thereof, —O-2-(2-nitrophenyl)prop-1-oxycarbonyl (“NPPOC”), —O-(2-nitrobenzyl) or a derivative or analog thereof, —O-(1-(2-nitrophenyl)ethyl) or a derivative or analog thereof, —O-1-(2-fluorophenyl)-4-methoxypiperidin-4-yl, —O-[(chloro-4-methyl)phenyl]-4′-methoxypiperidin-4-yl, tetrahydropyranyl ether, ethoxyethyl ether, methallyl ether, prenyl ether, or methoxymethyl ether;
A is CH;
PG² is H;
PG¹ is 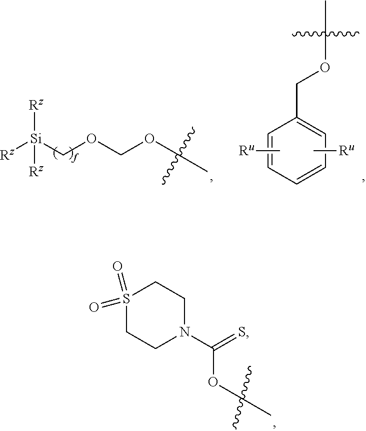 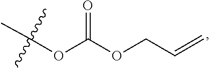 [SMILES: *OC(=O)OCC=C]
—O—CH₂—S—CH₃, —O—CH₂—N₃, or —O—CH₂—CH═CH₂; or
PG¹-A-PG² taken together form C(O)H, C(O)OMe, —C(O)OT, 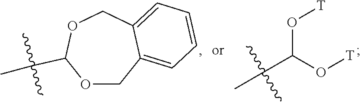
T is an C₁-C₆ branched or unbranched alkyl group;
f is 0 or 2;
each R^{u} is independently H, —O—C₁-C₄ alkyl or —C₁-C₄ alkyl; and
each R^{z} is independently a branched or unbranched C₁-C₆ alkyl group;
provided that when PG¹-A-PG² together form C(O)H, C(O)OMe, or 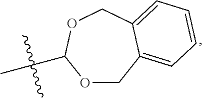 [SMILES: *C1OCc2ccccc2CO1]
R¹ and R² are both not —OH.

2. The compound of claim 1, wherein R² is: 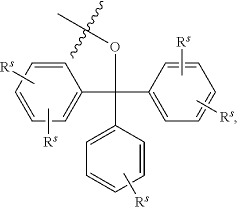
where each R^{s} is independently H, —O—C₁-C₄ alkyl or —C₁-C₄ alkyl.

3. The compound of claim 2, wherein R² is:

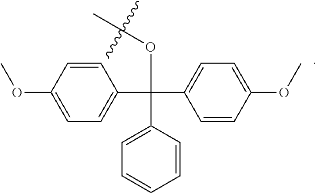

4. The compound of claim 3, wherein PG¹ is:

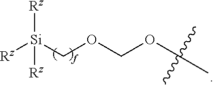

5. The compound of claim 4, wherein f is 2.

6. The compound of claim 4, wherein f is 0.

7. The compound of claim 4, wherein R¹ is —OH.

8. The compound of claim 4, wherein R¹ is —O—R^{w}—Z.

9. The compound of claim 8, wherein Z is alkyl, alkenyl, alkynyl, —CH₂-alkynyl, or acyl.

10. The compound of claim 8, wherein Z is alkyl.

11. The compound of claim 8, wherein Z is alkynyl or —CH₂-alkynyl.

12. The compound of claim 3, wherein PG¹ is:

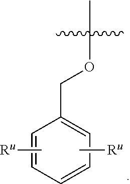

13. The compound of claim 12, wherein R¹ is —OH.

14. The compound of claim 12, wherein R¹ is —O—R^{w}—Z.

15. The compound of claim 14, wherein Z is alkyl, alkenyl, alkynyl, —CH₂-alkynyl, or acyl.

16. The compound of claim 14, wherein Z is alkyl.

17. The compound of claim 14, wherein Z is alkynyl or —CH₂-alkynyl.

18. The compound of claim 3, wherein PG¹ is:

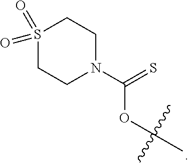 [SMILES: *OC(=S)N1CCS(=O)(=O)CC1]

19. The compound of claim 18, wherein R¹ is —OH.

20. The compound of claim 18, wherein R¹ is —O—R^{w}—Z.

21. The compound of claim 20, wherein Z is alkyl, alkenyl, alkynyl, —CH₂-alkynyl, or acyl.

22. The compound of claim 20, wherein Z is alkyl.

23. The compound of claim 20, wherein Z is alkynyl or —CH₂-alkynyl.

24. The compound of claim 3, wherein PG¹ is —O—CH₂—N₃ or —O—CH₂—S—CH₃.

25. The compound of claim 24, wherein R¹ is —OH.

26. The compound of claim 24, wherein R¹ is —O—R^{w}—Z.

27. The compound of claim 26, wherein Z is alkyl, alkenyl, alkynyl, —CH₂-alkynyl, or acyl.

28. The compound of claim 26, wherein Z is alkyl.

29. The compound of claim 26, wherein Z is alkynyl or —CH₂-alkynyl.

30. The compound of claim 3, wherein PG¹ is —O—CH₂—CH═CH₂.

31. The compound of claim 30, wherein R¹ is —OH.

32. The compound of claim 30, wherein R¹ is —O—R^{w}—Z.

33. The compound of claim 32, wherein Z is alkyl, alkenyl, alkynyl, —CH₂-alkynyl, or acyl.

34. The compound of claim 32, wherein Z is alkyl.

35. The compound of claim 32, wherein Z is alkynyl or —CH₂-alkynyl.

36. The compound of claim 3, wherein PG¹-A-PG² together form C(O)H, C(O)OMe, —C(O)OT,

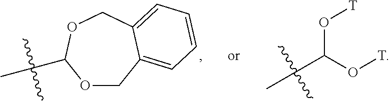

37. The compound of claim 36, wherein R¹ is —OH.

38. The compound of claim 36, wherein R¹ is —O—R^{w}—Z.

39. The compound of claim 38, wherein Z is alkyl, alkenyl, alkynyl, —CH₂-alkynyl, or acyl.

40. The compound of claim 38, wherein Z is alkyl.

41. The compound of claim 38, wherein Z is alkynyl or —CH₂-alkynyl.

42. The compound of claim 1, wherein R¹ and R² are both —OH, and wherein PG¹ is: 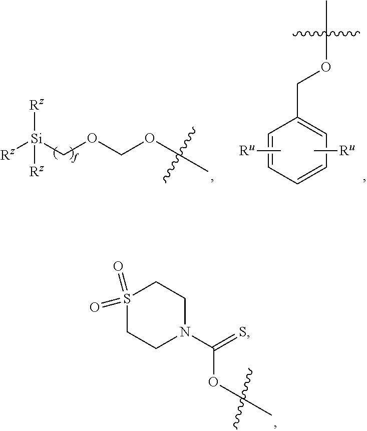
—O—CH₂—S—CH₃, —O—CH₂—N₃, or —O—CH₂—CH═CH₂.

43. The compound of claim 42, wherein PG¹ is 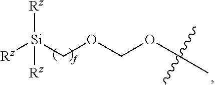
and wherein f is 0.

44. The compound of claim 42, wherein PG¹ is 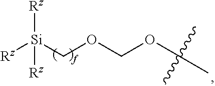
and wherein f is 2.

45. The compound of claim 42, wherein PG¹ is 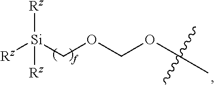
and wherein each R^{z} is a C₁-C₆ branched or unbranched alkyl group.

46. The compound of claim 42, wherein PG¹ is

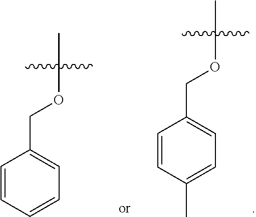

47. A compound of claims [symbol] , wherein the compound is regioisomerically and enantiomerically pure.

48. A compound having the structure of any one of Formulas (IIIA) or (IIIB): 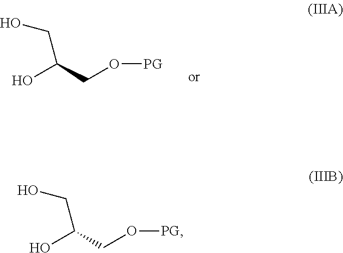
wherein
PG is 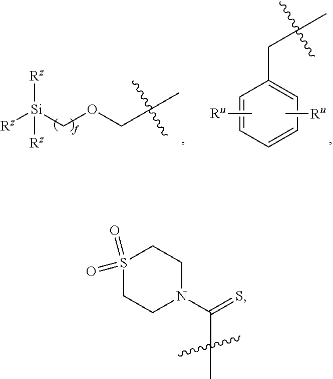
—CH₂—S—CH₃, —CH₂—N₃, or —CH₂—CH═CH₂;
f is 0 or 2;
each R^{u} is independently H, —O—C₁-C₄ or —C₁-C₄; and
each R^{z} is independently a branched or unbranched C₁-C₆ alkyl group.

49. The compound of claim 48, wherein PG is 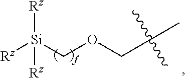
and wherein f is 2.

50. The compound of claim 48, wherein PG is 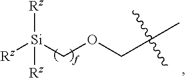
and wherein f is 0.

51. The compound of claim 48, wherein PG is 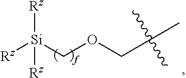
and wherein each R^{z} is a C₁-C₆ branched or unbranched alkyl group.

52. The compound of claim 48, wherein PG is

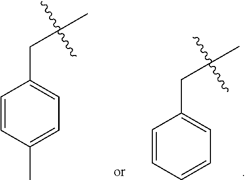

53. The compound of claim 48, wherein the compound is selected from the group consisting of:

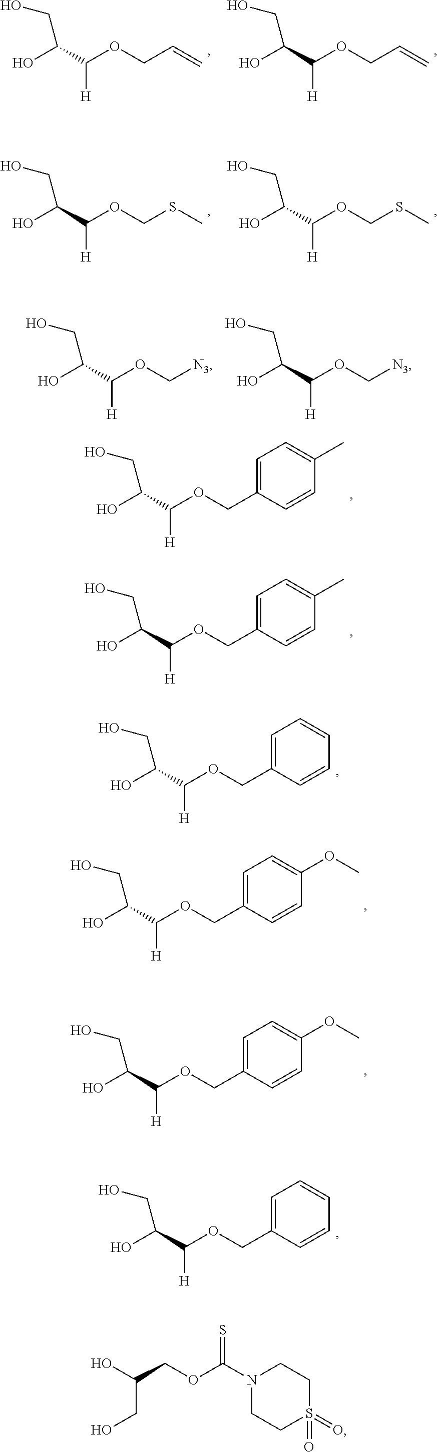

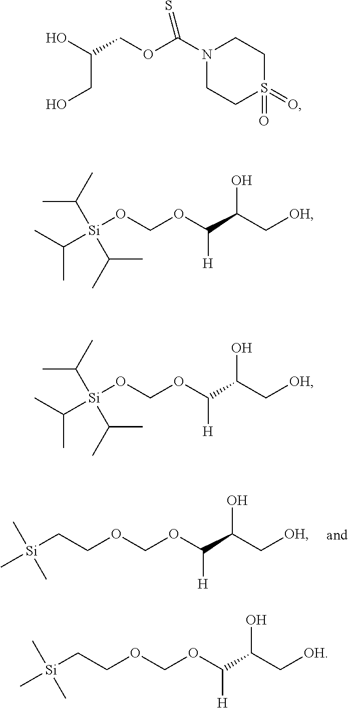

54. A compound having the structure of any one of Formulas (IVA) and (IVB): 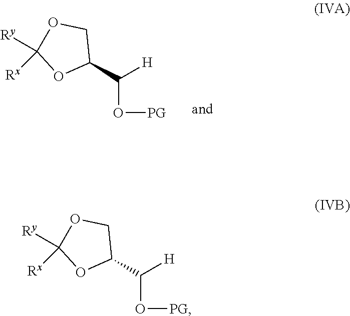
wherein
PG is 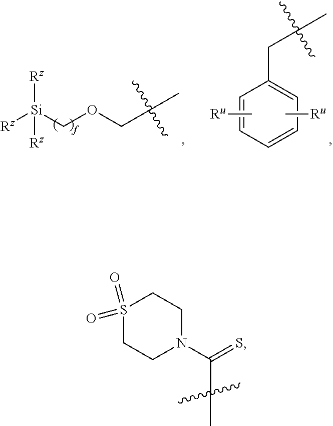
—CH₂—S—CH₃, —CH₂—N₃, or —CH₂—CH═CH₂;
f is 0 or 2;
each R^{u} is independently H, —O—C₁-C₄ or —C₁-C₄;
each R^{z} is independently a branched or unbranched C₁-C₆ alkyl group; and
R^{x} and R^{y} are independently a C₁-C₄ alkyl group.

55. The compound of claim 54, wherein R^{x} and R^{y} are both methyl.

56. The compound of claim 55, wherein PG is 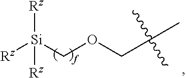
and wherein f is 2.

57. The compound of claim 55, wherein PG is 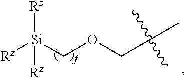
and wherein f is 0.

58. The compound of claim 55, wherein PG is 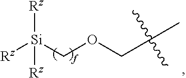
and wherein each R^{z} is a C₁-C₆ branched or unbranched alkyl group.

59. The compound of claim 55, wherein PG

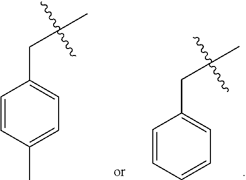

60. A compound having the structure of any one of Formulas (VA) and (VB): 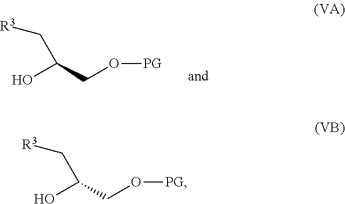
wherein
PG is 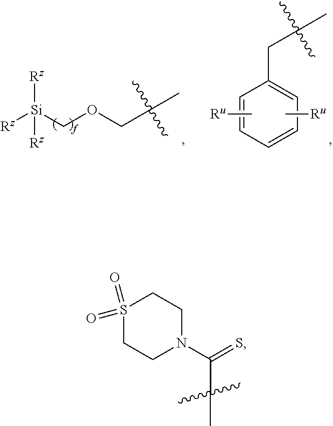
—CH₂—S—CH₃, —CH₂—N₃, or —CH₂—CH═CH₂;
R³ is 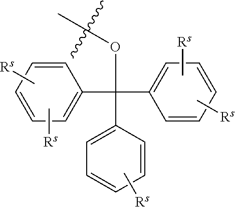
or —O-9-phenylthioxanthyl or a derivative or analog thereof;
f is 0 or 2;
each R^{u} is independently H, —O—C₁-C₄ alkyl or —C₁-C₄ alkyl;
each R^{s} is independently H, —O—C₁-C₄ alkyl or —C₁-C₄ alkyl; and
each R^{z} is independently a branched or unbranched C₁-C₆ alkyl group.

61. The compound of claim 60, wherein R³ is

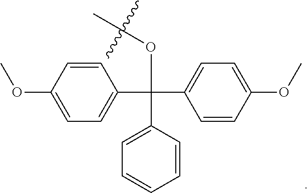

62. The compound of claim 60, wherein PG is 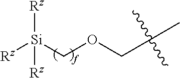
and wherein f is 0.

63. The compound of claim 60, wherein PG is 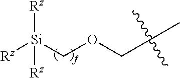
and wherein f is 2.

64. The compound of claim 60, wherein PG is 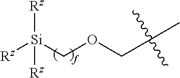
and wherein each R^{z} is a C₁-C₆ branched or unbranched alkyl group.

65. The compound of claim 60, wherein PG is

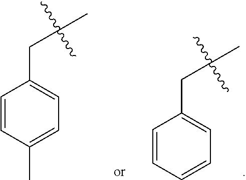

66. The compound of claim 60, wherein the compound is selected from the group consisting of: 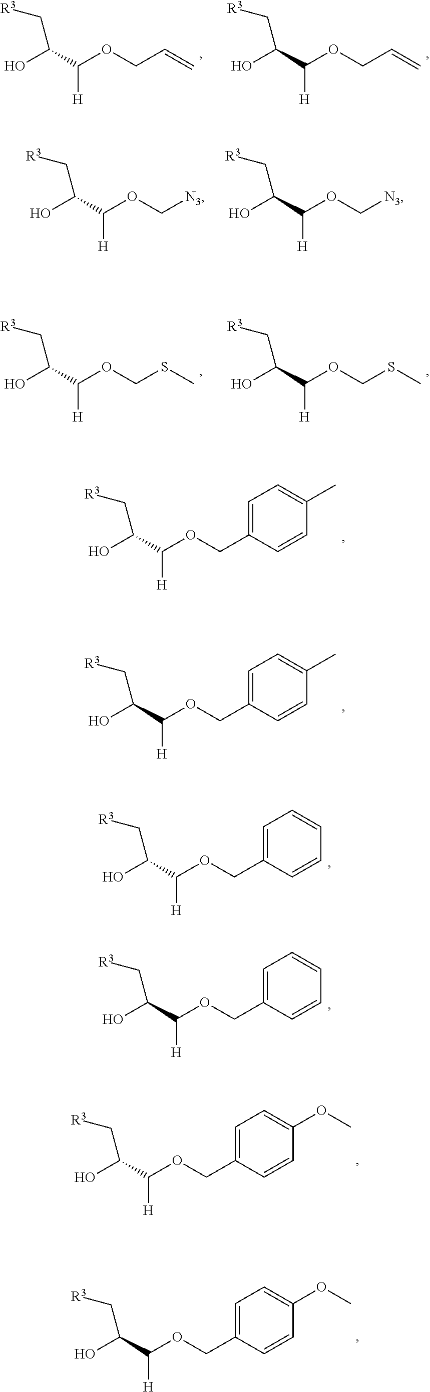 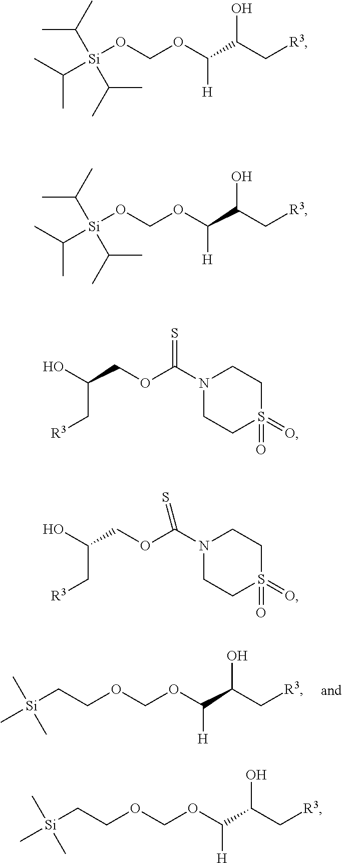
where R³ is 4,4′-dimethoxytrityl ether, 4-methoxytrityl ether, or —O-(9-phenylthioxanthyl).

67. A compound having the structure of any one of Formulas (VIIA) and (VIIB): 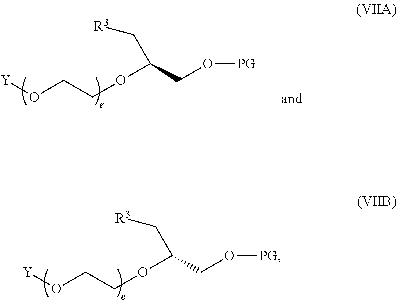
wherein
PG is 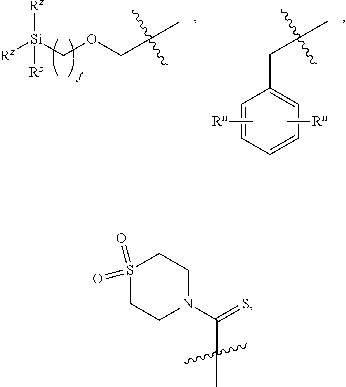
—CH₂—S—CH₃, —CH₂—N₃, or —CH₂—CH₂═CH₂;
R³ is 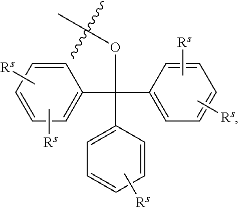
or —O-9-phenylthioxanthyl or a derivative or analog thereof;
Y is alkyl, alkenyl, alkynyl, acyl, —CH₂-alkynyl, a “click functional group,” -Het, or —CH₂-Het, where “Het” is a substituted or unsubstituted 5-membered heterocyclic moiety;
each R^{u} is independently H, —O—C₁-C₄ alkyl or —C₁-C₄ alkyl;
each R^{s} is independently H, —O—C₁-C₄ alkyl or —C₁-C₄ alkyl;
each R^{z} is independently a branched or unbranched C₁-C₆ alkyl group;
e is an integer ranging from between 1 to about 36; and
f is 0 or 2.

68. The compound of claim 67, wherein R³ is

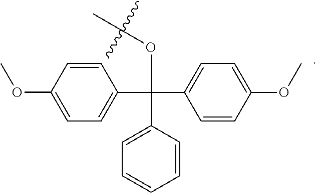

69. The compound of claim 67, wherein PG is 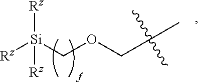
and wherein f is 0.

70. The compound of claim 67, wherein PG is 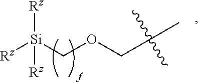
and wherein f is 2.

71. The compound of claim 67, wherein PG is 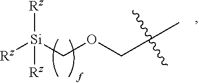
and wherein each R^{z} is a C₁-C₆ branched or unbranched alkyl group.

72. The compound of claim 67, wherein PG is

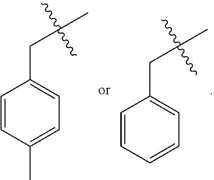

73. The compound of claim 67, wherein e ranges from 2 to 24.

74. The compound of claim 67, wherein e ranges from 2 to 12.

75. The compound of claim 67, wherein e is 3 or 4.

76. The compound of claim 67, wherein Y is alkyl.

77. The compound of claim 67, wherein Y is —CH₃.

78. The compound of claim 67, wherein Y is alkynyl or —CH₂-alkynyl.

79. The compound of claim 67, wherein Y is

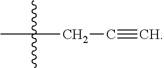 [SMILES: *CC#C]

80. The compound of claim 67, wherein Y includes either (i) -Het; or (ii) or —CH₂-Het, where “Het” is a substituted 5-membered heterocyclic moiety.

81. The compound of claim 80, wherein the 5-membered heterocyclic moiety is a triazole.

82. The compound of claim 81, wherein the triazole is substituted with an alkylaryl group.

83. A compound selected from the group consisting of: 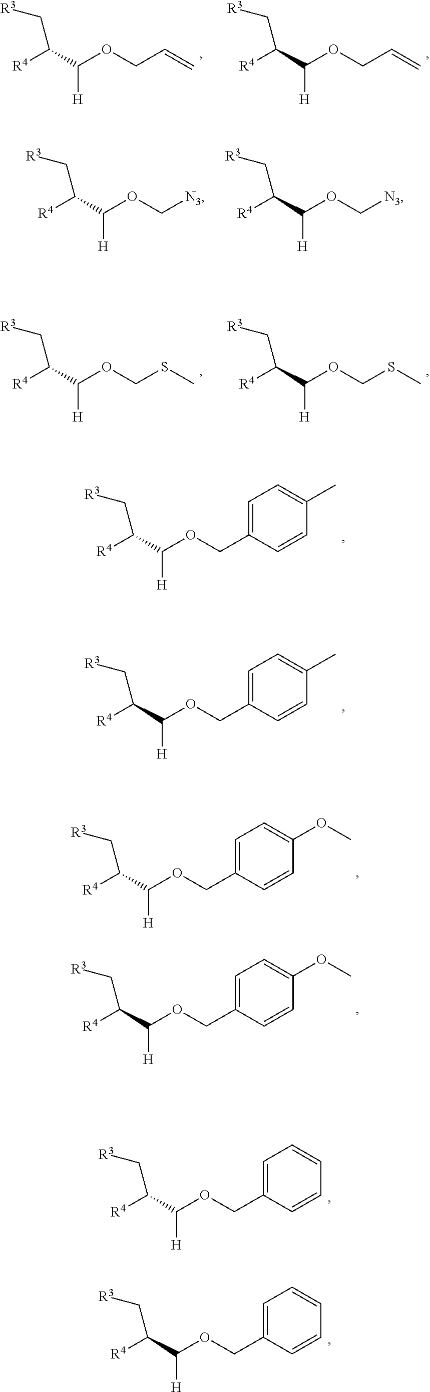 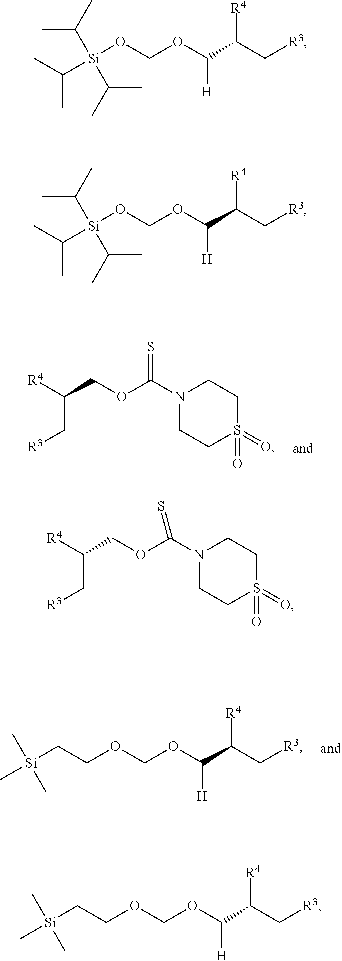
where R³ is 4,4′-dimethoxytrityl ether, 4-methoxytrityl ether, or —O-(9-phenylthioxanthyl);
R⁴ is —O-PEG₃-Y or —O-PEG₄-Y; and
Y is methyl, —CH═CH₂, 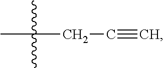 [SMILES: *CC#C]
-Het, or —CH₂-Het, where “Het” is a substituted or unsubstituted 5-membered heterocyclic moiety.

84. A compound of claim [symbol] , wherein the compound is regioisomerically and enantiomerically pure.

85. A compound having the structure of any one of Formulas (XA) and (XB): 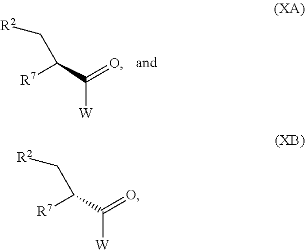
wherein
W is H, —O—CH₃ or —O-T, where T is a C₁-C₆ branched or unbranched alkyl group; and
R⁷ is —O—R^{w}—Z, where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, —CH₂— alkynyl, alkynyl, acyl, a “click functional group,” -Het, or —CH₂-Het, where “Het” is a substituted or unsubstituted 5-membered heterocyclic moiety.

86. The compound of claim 85, wherein R^{w} comprises between 2 and about 50 carbon atoms and which includes one or more oxygen heteroatoms.

87. The compound of claim 85, wherein R^{w} comprises between 2 and about 40 carbon atoms and which includes one or more oxygen heteroatoms.

88. The compound of claim 85, wherein R^{w} comprises between 2 and about 20 carbon atoms and which includes one or more oxygen heteroatoms.

89. The compound of claim 85, wherein Z is alkynyl or —CH₂-alkynyl.

90. The compound of claim 85, wherein Z is —CH₃.

91. The compound of claim 85, wherein Z includes either (i) -Het; or (ii) or —CH₂-Het; where “Het” is a substituted 5-membered heterocyclic moiety.

92. The compound of claim 85, wherein Z is a 5-membered heterocyclic moiety substituted with an alkylaryl group.

93. The compound of claim 92, wherein the alkylaryl group is —CH₂—CH₂—O-Bz.

94. The compound of claim 85, wherein R⁷ has any one of Formulas (VIIIA) or (VIIIB): 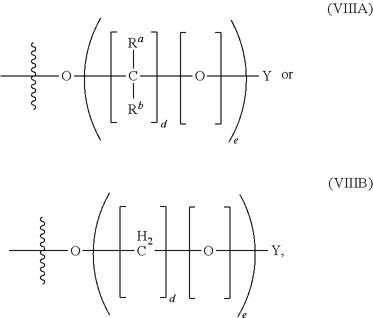
R^{a} and R^{b} are independently H or a C₁-C₄ alkyl;
Y is alkyl, alkenyl, alkynyl, acyl, —CH₂-alkynyl, a “click functional group,” -Het, or —CH₂-Het, where “Het” is a substituted or unsubstituted 5-membered heterocyclic moiety;
d is an integer ranging from between 1 to about 10; and
e is an integer ranging from between 1 to about 36.

95. The compound of claim 94, wherein d is 1 and e ranges from between 2 and 24.

96. The compound of claim 94, wherein d is 2 and e ranges from between 2 and 24.

97. The compound of claim 94, wherein at least one of R^{a} or R^{b} is —CH₃.

98. The compound of claim 94, wherein d is 1 or 2, e ranges from between 2 and 24, and Y is alkyl.

99. The compound of claim 98, wherein e is 3 or 4.

100. The compound of claim 94, wherein d is 1 or 2, e ranges from between 2 and 24, and Y is alkynyl or —CH₂-alkynyl.

101. The compound of claim 99, wherein e is 4.

102. The compound of claim 94, wherein d is 1 or 2, e ranges from between 2 and 24, and Y is acyl.

103. The compound of claim 94, wherein d is 1 or 2, e ranges from between 2 and 24, and Y is alkenyl.

104. The compound of claim 85, wherein the compound is selected from the group consisting of:

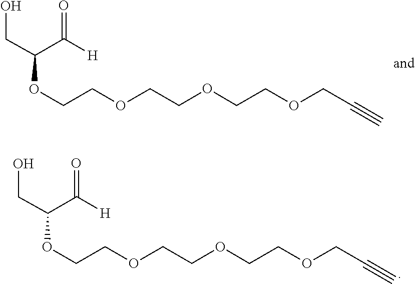 [SMILES: C#CCOCCOCCOCCO[C@H](C=O)CO ; C#CCOCCOCCOCCO[C@@H](C=O)CO]

105. A compounding having the structure of any one of Formulas (XIA) and (XIB): 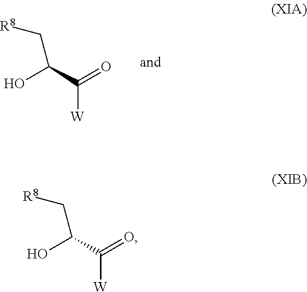
wherein
W is H, —O—CH₃ or —O-T, where T is an C₁-C₆ branched or unbranched alkyl group; and
R⁸ is —OH, —O-trityl or a derivative or analog thereof, —O-9-phenylthioxanthyl (pixyl) or a derivative or analog thereof, —O-2-(2-nitrophenyl)prop-1-oxycarbonyl (“NPPOC”), —O-(2-nitrobenzyl) or a derivative or analog thereof, —O-(1-(2-nitrophenyl)ethyl) or a derivative or analog thereof, —O-1-(2-fluorophenyl)-4-methoxypiperidin-4-yl, —O-[(chloro-4-methyl)phenyl]-4′-methoxypiperidin-4-yl, tetrahydropyranyl ether, ethoxyethyl ether, methallyl ether, prenyl ether, or methoxymethyl ether.

106. The compound of claim 105, wherein R⁸ is selected from the group consisting of: 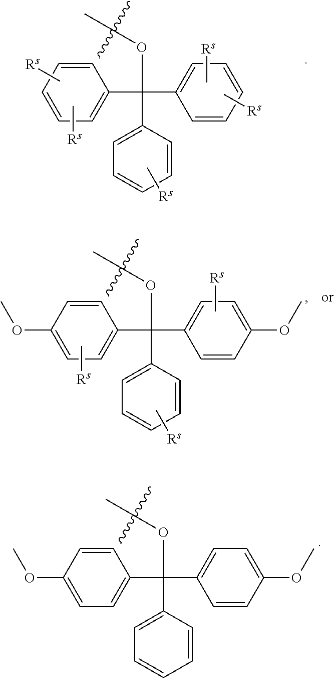
where each R^{s} is independently H, —O—C₁-C₄ alkyl or —C₁-C₄ alkyl.

107. The compound of claim 105, wherein R⁸ is:

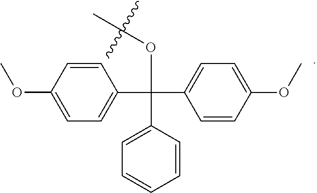

108. A compound selected from the group consisting of: 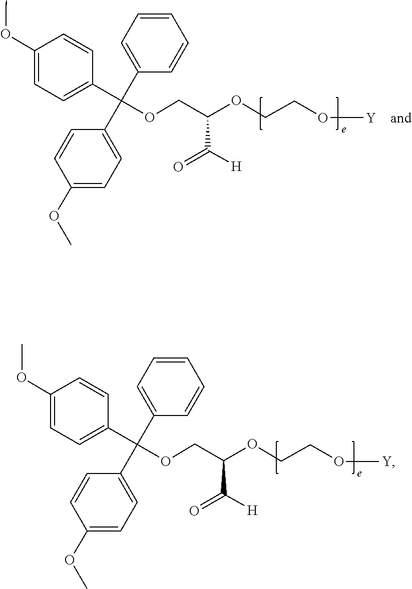
where e is an integer ranging from between 1 to about 24; and Y is alkyl, alkenyl, alkynyl, acyl, —CH₂-alkynyl, a “click functional group,” -Het, or —CH₂-Het, where “Het” is a substituted or unsubstituted 5-membered heterocyclic moiety.

109. The compound of claim 108, wherein e is 8.

110. The compound of claim 109, where Y is alkynyl or —CH₂-alkynyl.

111. The compound of claim 108, wherein e is 3 or 4.

112. The compound of claim 111, where Y is alkynyl or —CH₂-alkynyl.

113. The compound of claim 108, where e is 12.

114. A compound of claim [symbol] , wherein the compound is regioisomerically and enantiomerically pure.

115. A regioisomerically pure phosphoramidite-containing monomer derived from the regioisomerically pure compound of claim 47.

116. A polymer derived from the regioisomerically pure phosphoramidite-containing monomer of claim 115.

117. A copolymer derived from a first regioisomerically pure phosphoramidite-containing monomer of claim 115 and a second regioisomerically pure phosphoramidite-containing monomer of claim 115, wherein the first and second regioisomerically pure phosphoramidite-containing monomers are different.

118. An XNTP comprising the polymer of claim 116 or the copolymer of claim 117.

119. A regioisomerically pure phosphoramidite-containing monomer derived from the regioisomerically pure compound of claim 84.

120. A polymer derived from the regioisomerically pure phosphoramidite-containing monomer of claim 119.

121. A copolymer derived from a first regioisomerically pure phosphoramidite-containing monomer of claim 119 and a second regioisomerically pure phosphoramidite-containing monomer of claim 119, wherein the first and second regioisomerically pure phosphoramidite-containing monomers are different.

122. An XNTP comprising the polymer of claim 120 or the copolymer of claim 121.

123. A regioisomerically pure phosphoramidite-containing monomer derived from the regioisomerically pure compound of claim 114.

124. A polymer derived from the regioisomerically pure phosphoramidite-containing monomer of claim 123.

125. A copolymer derived from a first regioisomerically pure phosphoramidite-containing monomer of claim 123 and a second regioisomerically pure phosphoramidite-containing monomer of claim 123, wherein the first and second regioisomerically pure phosphoramidite-containing monomers are different.

126. An XNTP comprising the polymer of claim 124 or the copolymer of claim 125.

127. A method of synthesizing a regioisomerically and enantiomerically pure monomer having any one of Formulas (VIIIA) to (VIIID), the method comprising:
(a) preparing a compound having any one of Formulas (IIIA) and (IIIB) from 2,3-isopropylidene-sn-glycerol, wherein the compound having any one of Formulas (IIIA) and (IIB) has the structure: 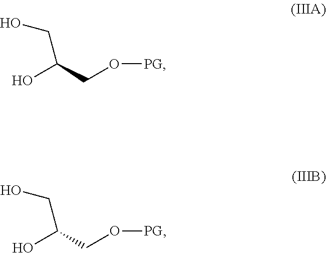
wherein
PG is 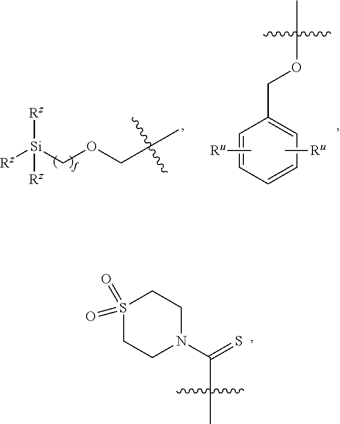
—CH₂—S—CH₃, —CH₂—N₃, or —CH₂—CH═CH₂;
f is 0 or 2;
each R^{u} is independently H, —O—C₁-C₄ alkyl or —C₁-C₄ alkyl; and
each R^{z} is independently a branched or unbranched C₁-C₆ alkyl group;
(b) reacting trityl chloride or a derivative or analog thereof with the compound having any one of Formulas (IIIA) and (IIIB) to provide a compound having any one of Formulas (VA) and (VB), respectively, wherein the compound having any one of Formulas (VA) and (VB) has the structure: 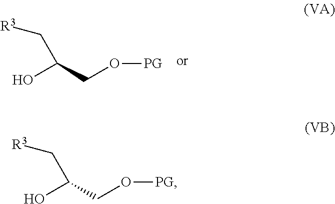
where R³ is —O-trityl or a derivative or analog thereof;
(c) coupling a reagent having Formula (XIV) with the compound having any one of Formulas (VA) and (VB) to provide a conjugate having any one of Formulas (VIA) and (VIB) respectively;
wherein the reagent having Formula (XIV) has the structure: 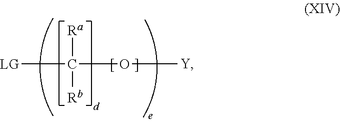
where
R^{a} and R^{b} are independently H or a C₁-C₄ alkyl;
Y is alkyl, alkenyl, alkynyl, acyl, —CH₂-alkynyl, a “click functional group,” -Het, or —CH₂-Het, where “Het” is a substituted or unsubstituted 5-membered heterocyclic moiety;
d is an integer ranging from between 1 to about 10;
e is an integer ranging from between 1 to about 36; and
LG is a leaving group;
wherein the conjugate having any one of Formulas (VIA) and (VIB) has the structure: 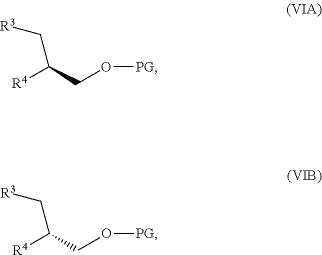
wherein R⁴ has Formula (XIIIA): 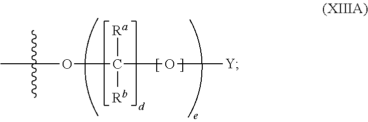
(d) deprotecting the conjugate having any one of Formulas (VIA) and (VIB) to provide the respective deprotected conjugate; and
(e) coupling a reagent having Formula (XV) to the deprotected conjugate to provide the regioisomerically and enantiomerically pure monomer having any one of Formulas (VIIIA) to (VIIID), respectively, wherein the compound having Formula (XV) has the structure: 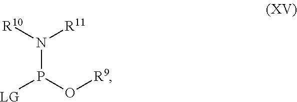
where LG is a leaving group, R⁹ is a substituted or unsubstituted C₁-C₆ alkyl group terminating in a cyano moiety; and where R¹⁰ and R¹¹ are independently a branched or unbranched C₁-C₆ alkyl group;
and where the compound having any one of Formulas (VIIIA) to (VIID) has the structure: 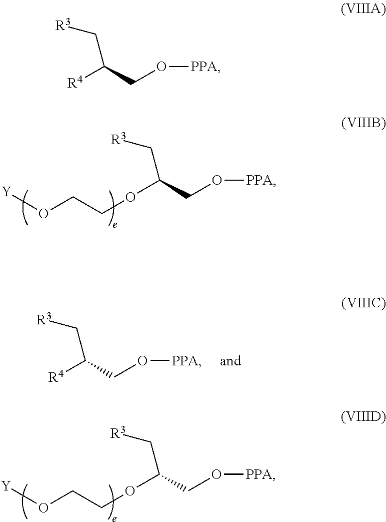
where PPA is —P(O—R⁹)—N(R¹⁰)(R¹¹).

128. The method of claim 127, wherein the preparation of the compound having any one of Formulas (IIIA) and (IIIB) from 2,3-isopropylidene-sn-glycerol comprises introducing a protecting group to 2,3-isopropylidene-sn-glycerol.

129. The method of claim 128, wherein the protecting group has the formula X-PG, where X is a leaving group, and where PG is: 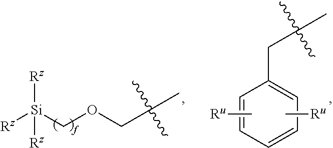
—CH₂—S—CH₃, —CH₂—CH═CH₂.

130. The method of claim 127, further comprising coupling a substituted or unsubstituted heterocyclic moiety to either (i) the deprotected conjugate having any one of Formulas (VIA) and (VIB); or (ii) the protected conjugate having any one of Formulas (VIA) and (VIB).

131. The method of claim 127, further comprising polymerizing the regioisomerically pure monomer having any one of Formulas (VIIIA) to (VIIID).

132. An oligomer derived from the regioisomerically and enantiomerically pure monomer prepared according to the process of claim 127.

133. A polymer derived from the regioisomerically and enantiomerically pure monomer prepared according to the process of claim 127.

134. A copolymer derived from a first regioisomerically and enantiomerically pure monomer and a second regioisomerically and enantiomerically pure monomer, wherein the first and second regioisomerically and enantiomerically pure monomers are each prepared according to the process of claim 127, and wherein the first and second regioisomerically and enantiomerically pure monomers are different.

135. A method of preparing a regioisomerically and enantiomerically pure compound having any one of Formulas (VA) and (VB) comprising:
a) obtaining a compound having any one of Formulas (IIIA) and (IIIB), wherein the compound having any one of Formulas (IIIA) and (IIIB) has the structure: 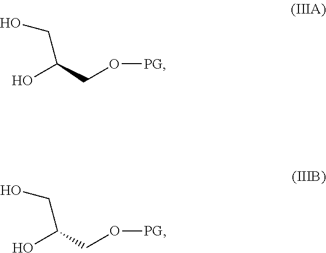
wherein
PG is 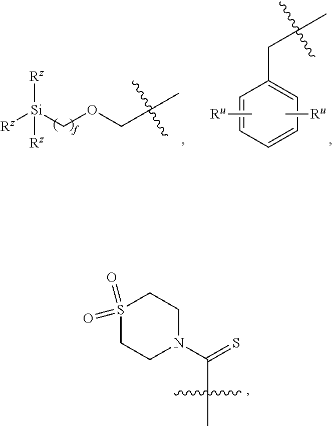
—CH₂—S—CH₃, —CH₂—N₃, or —CH₂—CH═CH₂;
f is 0 or 2;
each R^{u} is independently H, —O—C₁-C₄ alkyl or —C₁-C₄ alkyl; and
each R^{z} is independently a branched or unbranched C₁-C₆ alkyl group;
b) reacting the obtained compound having any one of Formulas (IIIA) and (IIIB) with trityl chloride or a derivative or analog thereof to provide the compound having any one of Formulas (VA) and (VB), wherein the compound having any one of Formulas (VA) and (VB) have the structure: 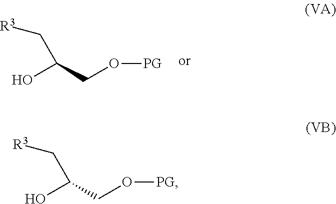
wherein R³ is —O-trityl or a derivative or analog thereof.

136. A method of preparing a regioisomerically and enantiomerically pure compound having any one of Formulas (VIA) and (VIB), comprising:
(a) obtaining a compound having any one of Formulas (VA) and (VB), wherein the compound having any one of Formulas (VA) and (VB) has the structure: 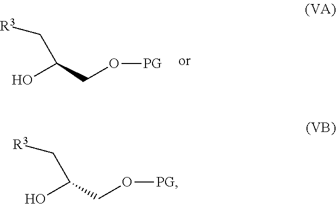
wherein
PG is 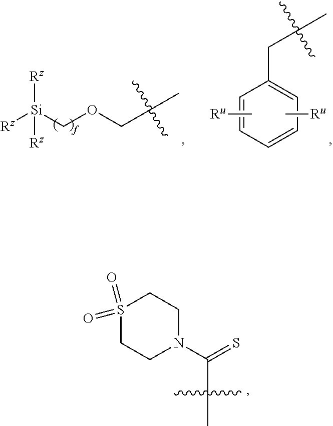
—CH₂—S—CH₃, —CH₂—N₃, or —CH₂—CH═CH₂;
f is 0 or 2;
each R^{u} is independently H, —O—C₁-C₄ alkyl or —C₁-C₄ alkyl;
each R^{z} is independently a branched or unbranched C₁-C₆ alkyl group; and
R³ is —O-trityl or a derivative or analog thereof or —O— phenylthioxanthyl or a derivative or analog thereof; and
(b) reacting the obtained compound having any one of Formulas (VA) and (VB) with a reagent having Formula (XIV) to provide a regioisomerically and enantiomerically pure compound having any one of Formulas (VIA) and (VIB) respectively,
wherein the reagent having Formula (XIV) has the structure: 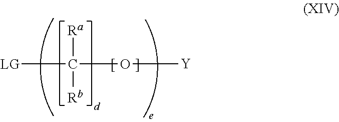
where
R^{a} and R^{b} are independently H or a C₁-C₄ alkyl;
Y is alkyl, alkenyl, alkynyl, acyl, —CH₂-alkynyl, a “click functional group,” -Het, or —CH₂-Het, where “Het” is a substituted or unsubstituted 5-membered heterocyclic moiety;
d is an integer ranging from between 1 to about 10;
e is an integer ranging from between 1 to about 36; and
LG is a leaving group; and
wherein the compound having any one of Formulas (VIA) and (VIB) has the structure: 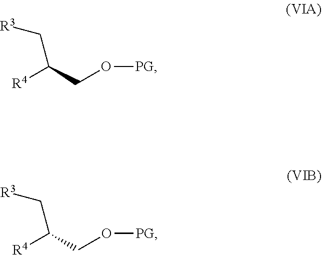
where R⁴ has Formula (XIIIA):

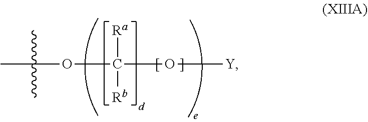

137. The method claim 136, further comprising deprotecting the regioisomerically and enantiomerically pure compound having any one of Formulas (VIA) and (VIB).

138. The method of claim 137, further comprising reacting the deprotected regioisomerically and enantiomerically pure compound with a reagent having Formula (XV) to provide a regioisomerically and enantiomerically pure compound having any one of Formulas (VIIIA) to (VIIID), where the reagent having Formula (XV) has the structure: 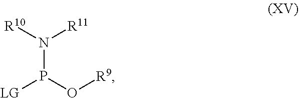
where LG is a leaving group, R⁹ is a substituted or unsubstituted C₁-C₆ alkyl group terminating in a cyano moiety; and where R¹⁰ and R¹¹ are independently a branched or unbranched C₁-C₆ alkyl group; and
where the regioisomerically and enantiomerically pure compound having any one of Formulas (VIIIA) to (VIIID) has the structure: 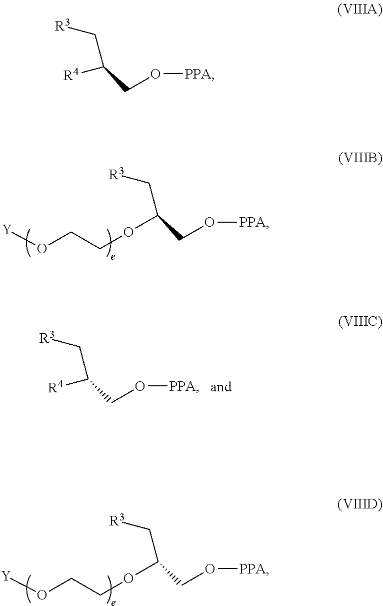
where PPA is —P(O—R⁹)—N(R¹⁰)(R¹¹).

139. A compound selected from the group consisting of: 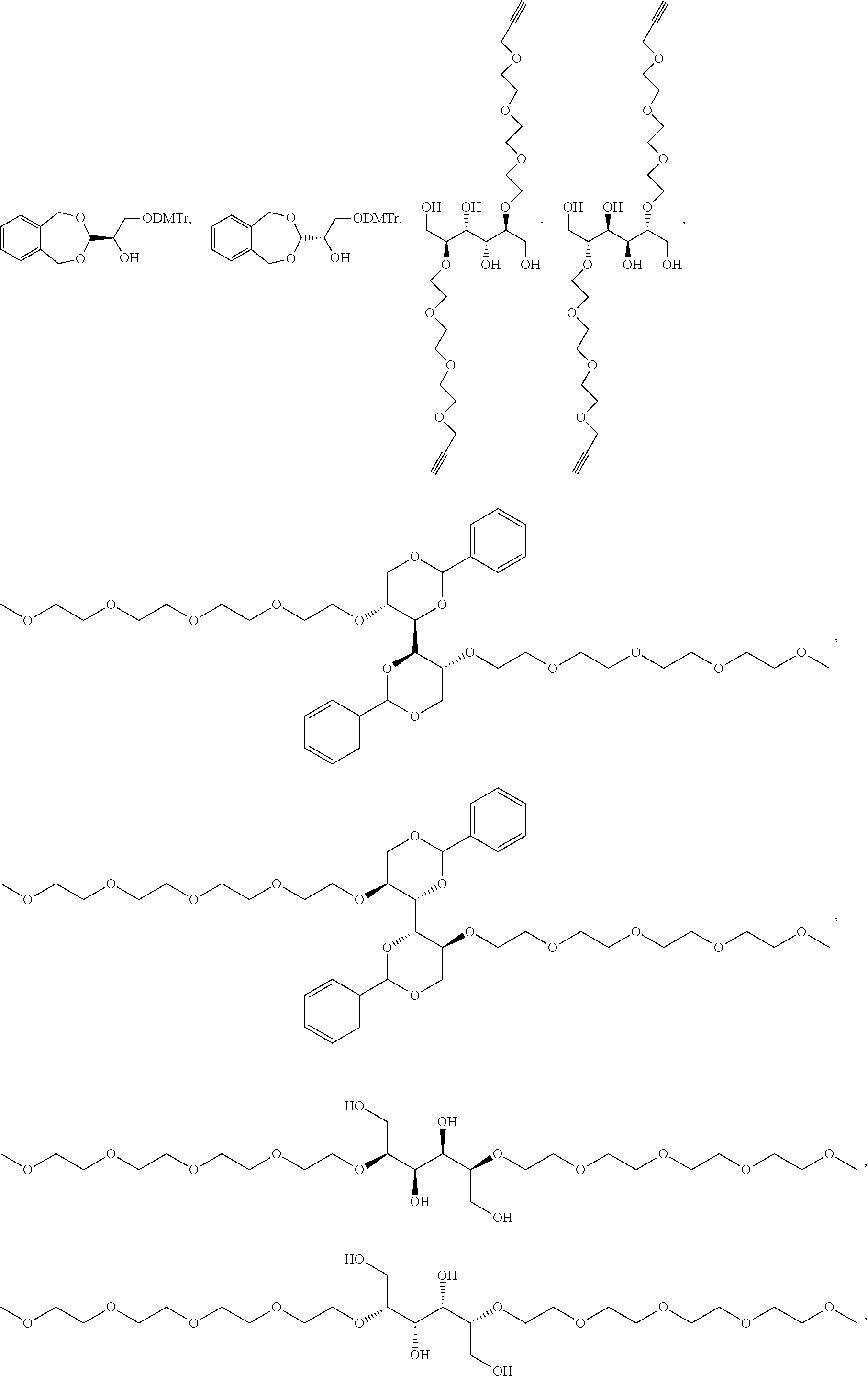 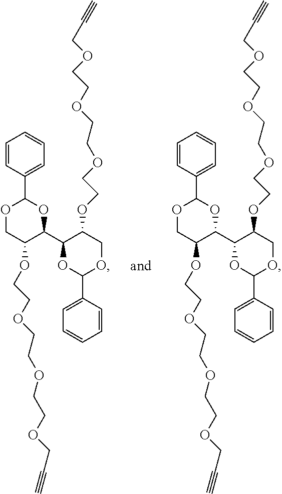
where ODMTr is 4,4′-dimethoxytrityl ether.

140. A compound selected from the group consisting of: 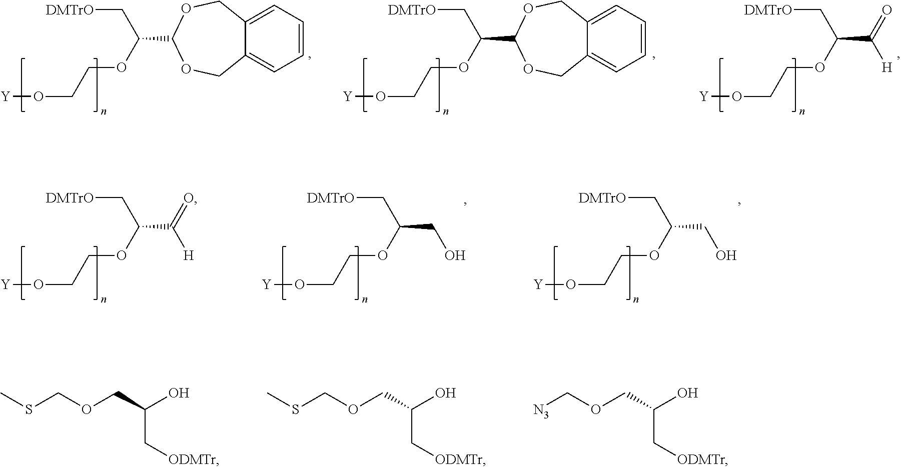 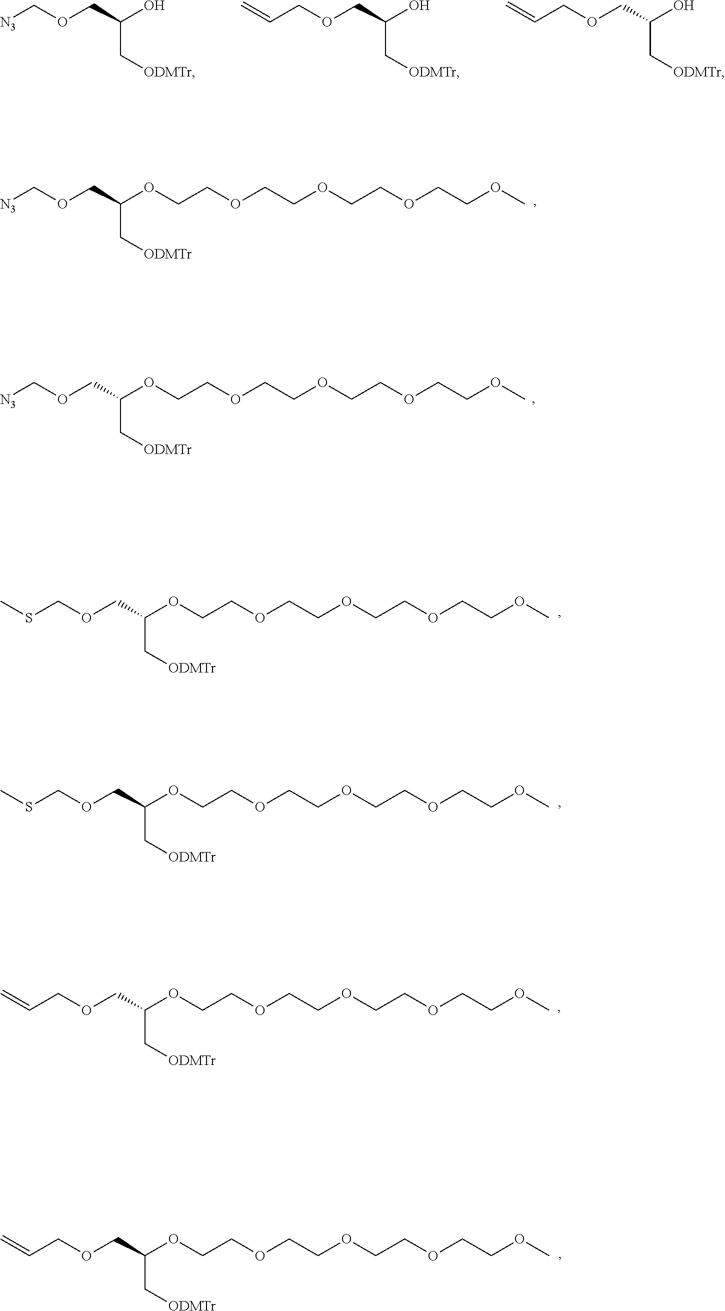 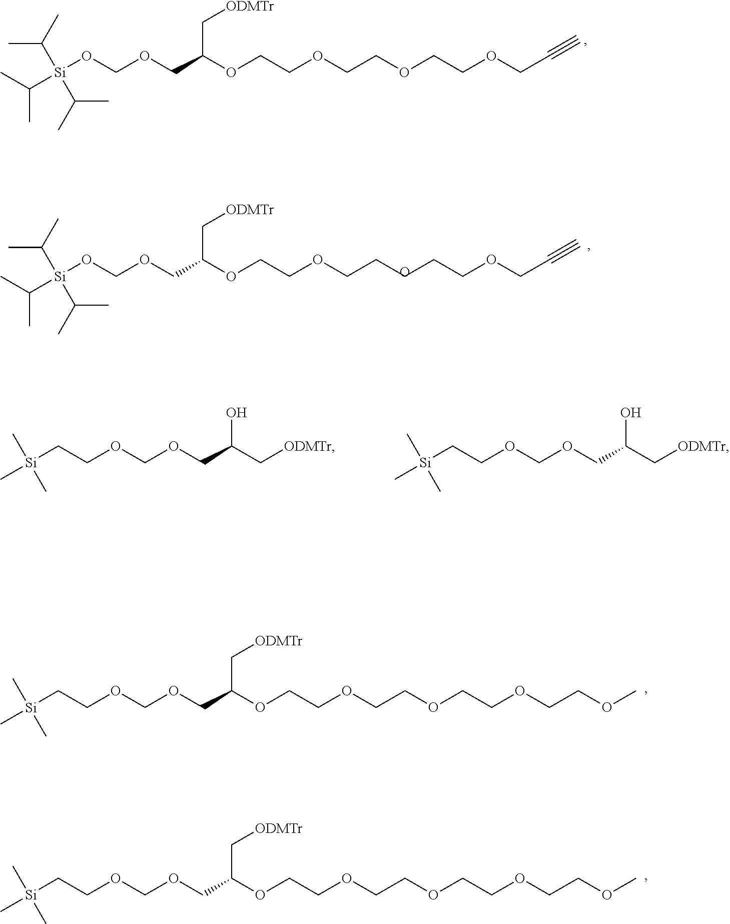 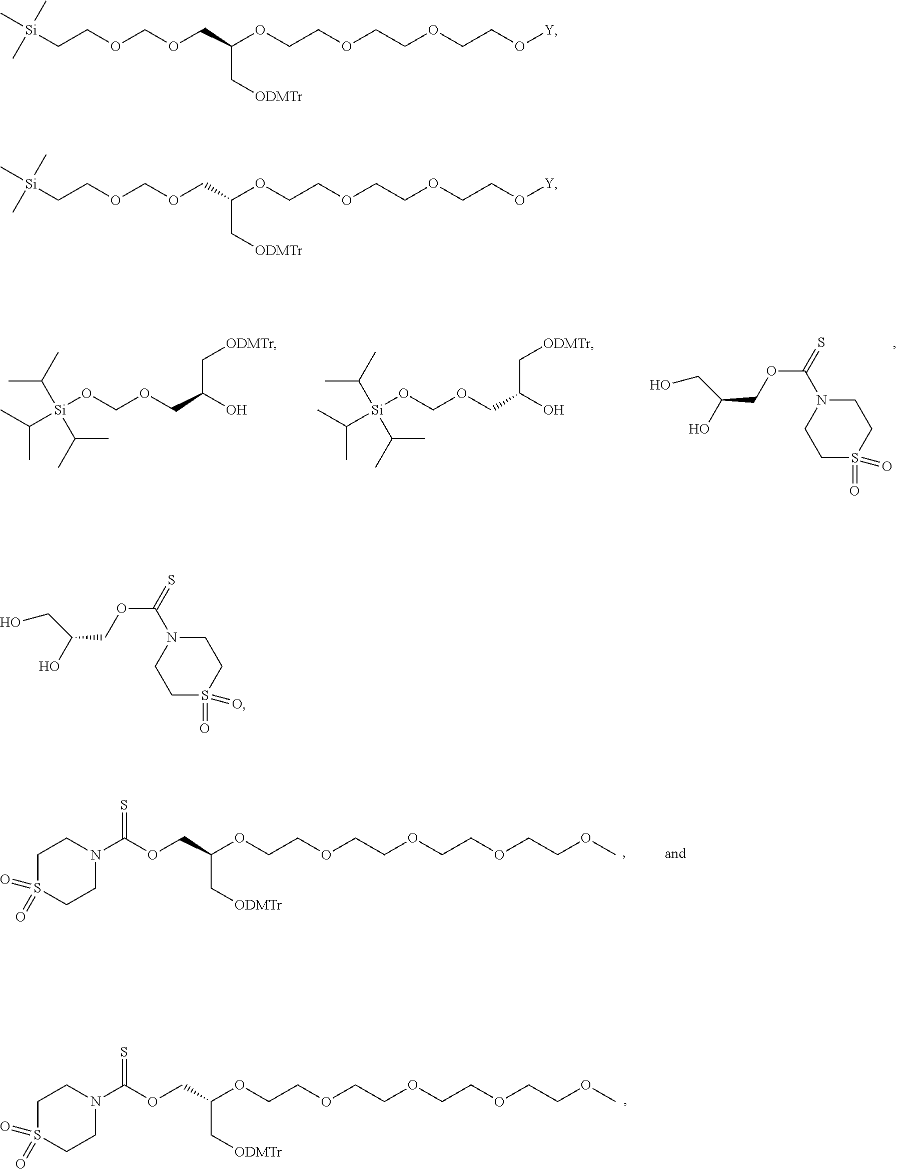
where n ranges from 1 to 24;
Y is alkyl, alkenyl, alkynyl, acyl, —CH₂-alkynyl, a “click functional group,” -Het, or —CH₂-Het, where “Het” is a substituted or unsubstituted 5-membered heterocyclic moiety; and
-ODMTr is 4,4′-dimethoxytrityl ether.

141. A compound having any one of Formulas (XIIIA) or (XIIIB): 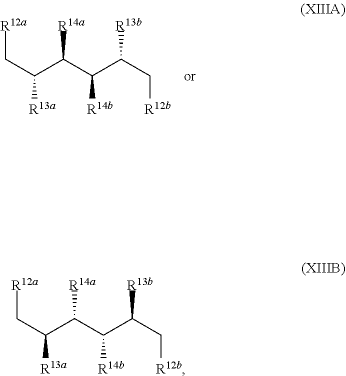
wherein
R^{12a} and R^{12b} are independently —OH, —O-trityl or a derivative or analog thereof, —O-9-phenylthioxanthyl (pixyl) or a derivative or analog thereof, —O-2-(2-nitrophenyl)prop-1-oxycarbonyl (“NPPOC”), —O-(2-nitrobenzyl) or a derivative or analog thereof, —O-(1-(2-nitrophenyl)ethyl) or a derivative or analog thereof, tetrahydropyranyl ether, ethoxyethyl ether, methallyl ether, prenyl ether, or methoxymethyl ether;
R^{13a} and R^{13b} are independently —O—R^{w}—Z, where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, or —CH₂-Het, where “Het” is a substituted or unsubstituted 5-membered heterocyclic moiety; and
R^{14a} and R^{14b} are —OH;
or where R^{12a} and R^{14a} and/or R^{12b} and R^{14b} taken together may be

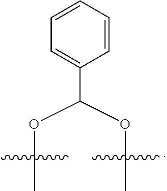 [SMILES: *OC(O*)c1ccccc1]

142. The compound of claim 141, wherein R^{12a} and R^{12b} are each independently —O-trityl or a derivative or analog thereof.

143. The compound of claim [symbol] , wherein Z is alkyl, alkenyl, alkynyl, or —CH₂-alkynyl.

144. The compound of claim [symbol] , wherein R^{w} includes between 4 and 16 carbon atoms.

145. The compound of claim [symbol] , wherein R^{w} includes between 6 and 12 carbon atoms.

146. The compound of claim [symbol] , wherein R^{w} includes between 8 and 12 carbon atoms.

147. The compound of claim [symbol] , wherein R^{w} further includes at least two oxygen heteroatoms.

148. The compound of claim 141, wherein R^{12a} and R^{12b} are each independently —OH.

149. The compound of claim 148, wherein Z is alkyl, alkenyl, alkynyl, or —CH₂-alkynyl.

150. The compound of claim 149, wherein R^{w} includes between 4 and 16 carbon atoms.

151. The compound of claim 149, wherein R^{w} includes between 6 and 12 carbon atoms.

152. The compound of claim 149, wherein R^{w} includes between 8 and 12 carbon atoms.

153. The compound of claim [symbol] , wherein R^{w} further includes at least two oxygen heteroatoms.

154. The compound of claim 141, wherein R^{12a} and R^{14a} taken together are 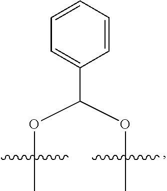 [SMILES: *OC(O*)c1ccccc1]
R¹² and R^{14b} taken together are 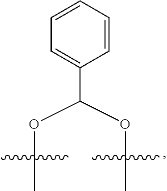 [SMILES: *OC(O*)c1ccccc1]
and where Z is alkyl, alkenyl, alkynyl, or —CH₂— alkynyl.

155. The compound of claim 154, wherein R^{w} includes between 4 and 16 carbon atoms.

156. The compound of claim 154, wherein R^{w} includes between 6 and 12 carbon atoms.

157. The compound of claim 154, wherein R^{w} includes between 8 and 12 carbon atoms.

158. The compound of claim [symbol] , wherein R^{w} further includes at least two oxygen heteroatoms.

159. A compound selected from the group consisting of:

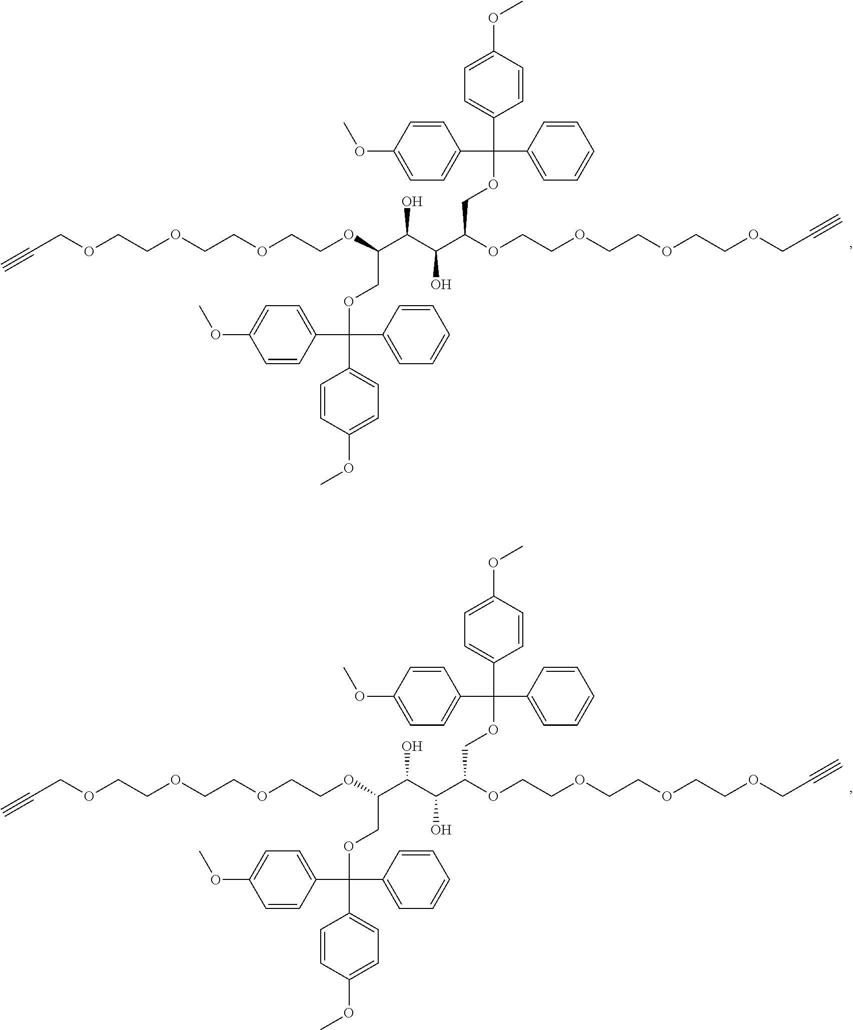

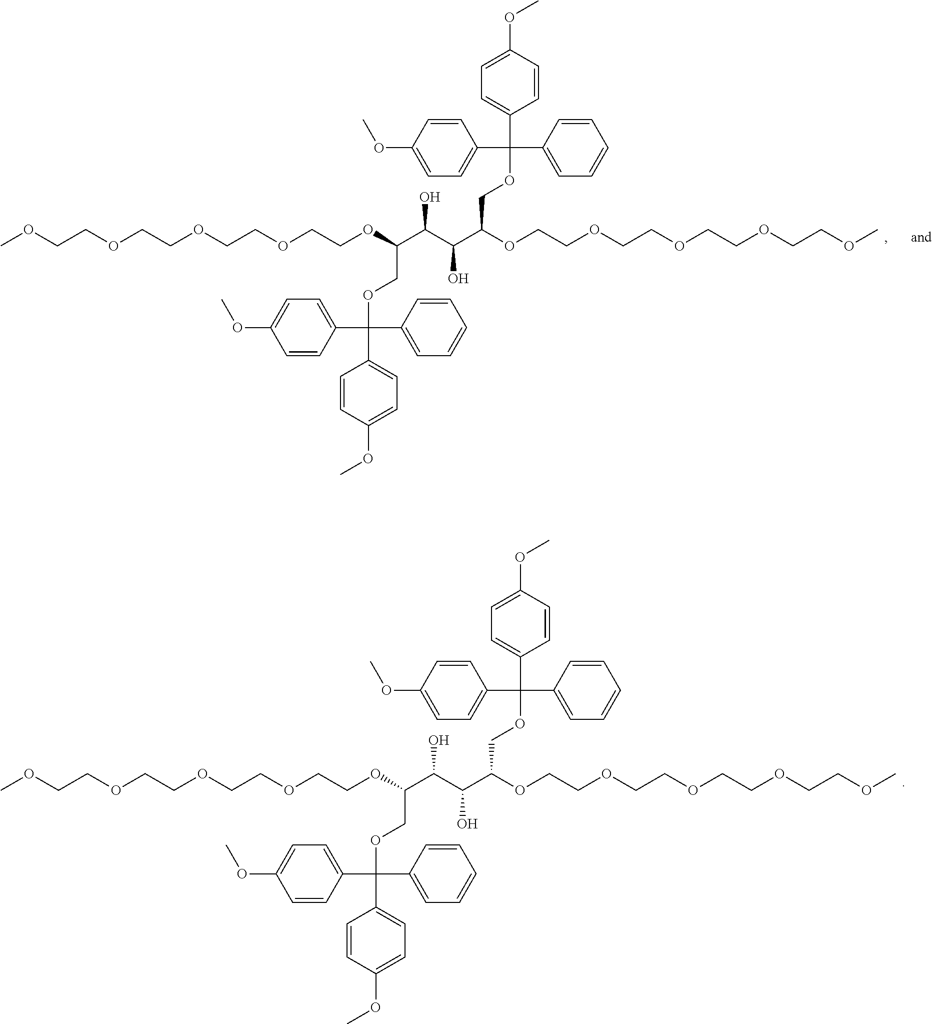

160. A method of preparing a compound having any one of the structures: 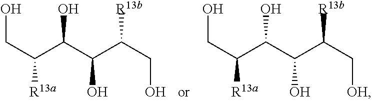
where R^{13a} and R^{13b} are independently —O—R^{w}—Z, and where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, or —CH₂-Het, where “Het” is a substituted or unsubstituted 5-membered heterocyclic moiety;
the method comprising:
introducing a lower alcohol and an acid to a starting material having any one of the structures: 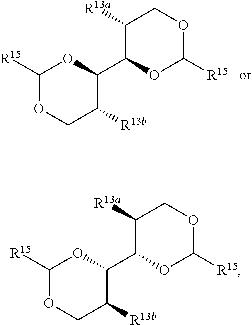
where R¹⁵ is a substituted or unsubstituted 5- or 6-membered aromatic or heteroaromatic group.

161. The method of claim 160, wherein the lower alcohol is selected from the group consisting of methanol and ethanol.

162. The method of claim [symbol] , wherein the acid is hydrochloric acid.

163. The method of claim [symbol] , wherein Z is alkyl, alkenyl, alkynyl, or —CH₂-alkynyl.

164. The method of claim [symbol] , wherein R^{w} includes between 4 and 16 carbon atoms.

165. The method of claim [symbol] , wherein R^{w} includes between 6 and 12 carbon atoms.

166. The method of claim [symbol] , wherein R^{w} includes between 8 and 12 carbon atoms.

167. The method of claim [symbol] , wherein R^{w} further includes at least two oxygen heteroatoms.

168. A method of preparing a compound having any one of the structures: 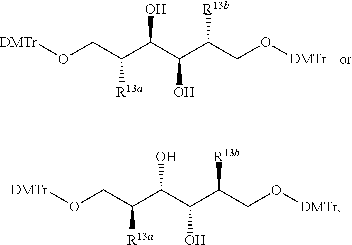
wherein R^{13a} and R^{13b} are independently —O—R^{w}—Z, and where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, or —CH₂-Het, where “Het” is a substituted or unsubstituted 5-membered heterocyclic moiety;
the method comprising:
reacting 4,4′Dimethoxytrityl chloride with a compound having any one of the following structures:

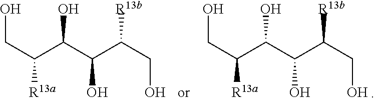

169. The method of claim 168, wherein Z is alkyl, alkenyl, alkynyl, —CH₂-alkynyl.

170. The method of claim [symbol] , wherein R^{w} includes between 4 and 16 carbon atoms.

171. The method of claim [symbol] , wherein R^{w} includes between 6 and 12 carbon atoms.

172. The method of claim [symbol] , wherein R^{w} includes between 8 and 12 carbon atoms.

173. The method of claim [symbol] , wherein R^{w} further includes at least two oxygen heteroatoms.

174. A method of preparing a compound having any one of Formulas (IXA) and (IXB), respectively: 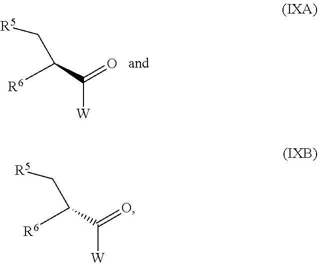
where
W is H;
R⁵ is —O-trityl;
R⁶—O—R^{w}—Z, where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂— alkynyl acyl, a “click functional group,” -Het, or —CH₂-Het, where “Het” is a substituted or unsubstituted 5-membered heterocyclic moiety;
the method comprising oxidatively cleaving a compound having any one of the structures: 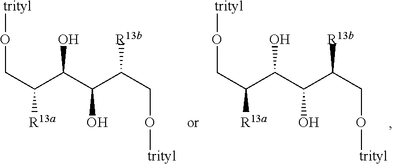
wherein R^{13a} and R^{13b} are independently —O—R^{w}—Z, and where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, or —CH₂-Het, where “Het” is a substituted or unsubstituted 5-membered heterocyclic moiety;
wherein the oxidative cleavage is performed in the presence sodium metaperiodate (NaIO₄) and a base; and wherein R⁶, R^{13a}, and R^{13b} are the same.

175. The method of claim 174, wherein the base is pyridine.

176. The method of claim [symbol] , wherein Z is alkyl, alkenyl, alkynyl, —CH₂-alkynyl.

177. The method of claim [symbol] , wherein R^{w} includes between 4 and 16 carbon atoms.

178. The method of claim [symbol] , wherein R^{w} includes between 6 and 12 carbon atoms.

179. The method of claim [symbol] , wherein R^{w} includes between 8 and 12 carbon atoms.

180. The method of claim [symbol] , wherein R^{w} further includes at least two oxygen heteroatoms.

181. A compound having any one of Formulas (IXA) and (IXB), respectively: 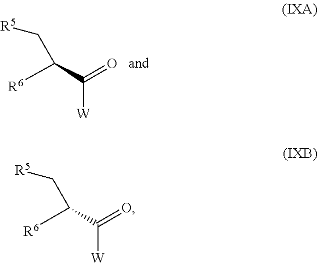
where
W is H, —O—CH₃, or —O-T, where T is an C₁-C₆ branched or unbranched alkyl group;
R⁵ is —OH, —O-trityl or a derivative or analog thereof, —O-9-phenylthioxanthyl (pixyl) or a derivative or analog thereof, —O-2-(2-nitrophenyl)prop-1-oxycarbonyl (“NPPOC”), —O-(2-nitrobenzyl) or a derivative or analog thereof, —O-(1-(2-nitrophenyl)ethyl) or a derivative or analog thereof, tetrahydropyranyl ether, ethoxyethyl ether, methallyl ether, prenyl ether, or methoxymethyl ether;
R⁶ is —OH, —O—R^{w}—Z, where R^{w} is a substituted or unsubstituted, branched or unbranched, saturated or unsaturated alkyl group having between 1 and 100 carbon atoms, and which optionally includes one or more oxygen heteroatoms, and where Z is alkyl, alkenyl, alkynyl, —CH₂-alkynyl, acyl, a “click functional group,” -Het, or —CH₂-Het, where “Het” is a substituted or unsubstituted 5-membered heterocyclic moiety;
provided that R⁵ and R⁶ are both not —OH.

182. The compound of claim 181, wherein W is —H.

183. The compound of claim 182, wherein R⁵ is —O-trityl or a derivative or analog thereof.

184. The compound of claim [symbol] , wherein R⁶ is —O—R^{w}—Z.

185. The compound of claim 184, wherein Z is alkyl, alkenyl, alkynyl, or —CH₂-alkynyl.

186. The compound of claim 185, wherein R^{w} includes between 4 and 16 carbon atoms.

187. The compound of claim 185, wherein R^{w} includes between 6 and 12 carbon atoms.

188. The compound of claim 185, wherein R^{w} includes between 8 and 12 carbon atoms.

189. The compound of claim [symbol] , wherein R^{w} further includes at least two oxygen heteroatoms.

190. The compound of claim 181, wherein the compound is selected from the group consisting of:

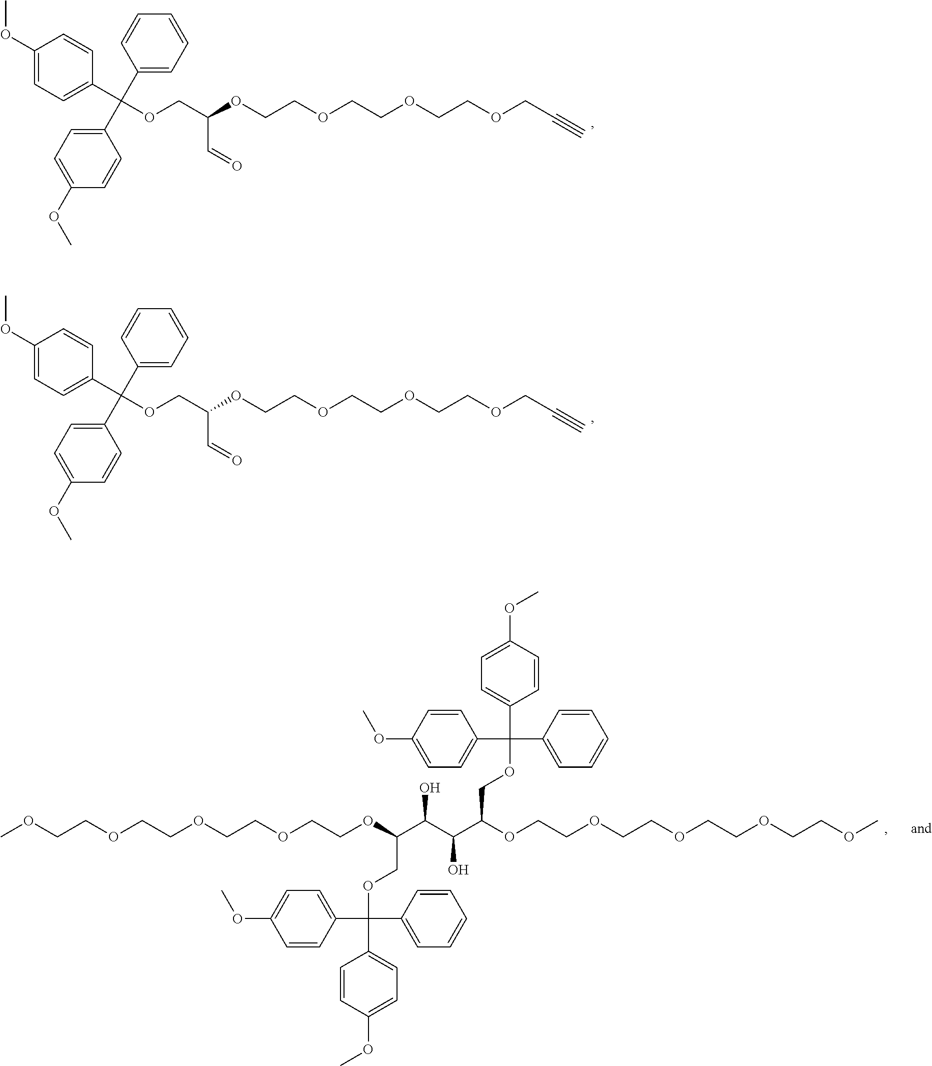

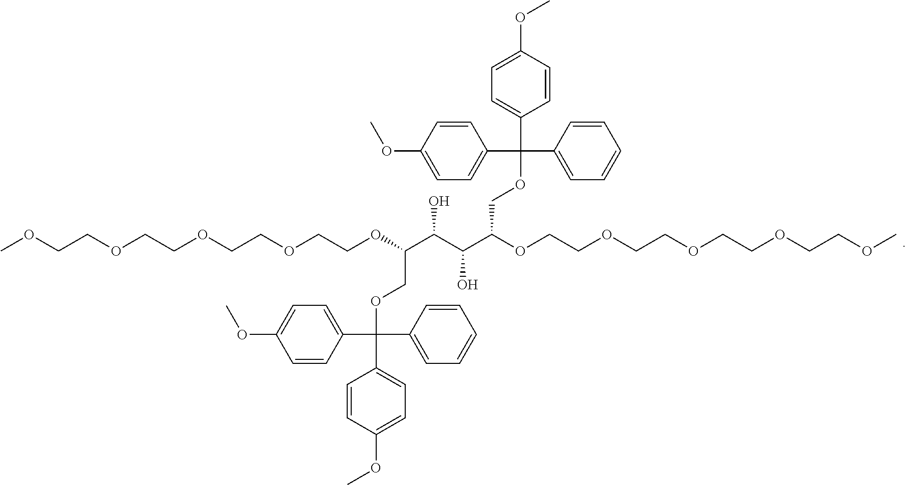

191. A regioisomerically pure phosphoramidite-containing monomer derived from the regioisomerically pure compound of claim [symbol] .

192. A polymer derived from the regioisomerically pure phosphoramidite-containing monomer of claim 191.

193. A copolymer derived from a first regioisomerically pure phosphoramidite-containing monomer of claim 191 and a second regioisomerically pure phosphoramidite-containing monomer of claim 191, wherein the first and second regioisomerically pure phosphoramidite-containing monomers are different.

194. An XNTP comprising the polymer of claim 192 or the copolymer of claim 193.
